SEQUENCE LISTING (73 sequences)

SEQ ID NO: 1 - DNA, 2308 bp, Homo sapiens
aggcgtgagccaccgcacccagccttttatttttatttatttatttattttttgagacagagtctccctctgtcacccaggctagagtgctgtggcaagattatggctaattgcagcctcttaactcctagactcaagagatcctcccacctcagcctcccaagtagctgggattacaggcacgtgccaccacacccagtgaattatttttgtagagatagggtcttgccatgttgcccaggctggtccccaactctgggctcaagcgatcctcctgccttgggctcccaaagccctggattacaggtgtgagccaccacactcagcccttggattggataattcttttggtgggggctgtgccttgaaggatgttagcagcatccttggcatctaatactaaatgccagtagccagtagtatctcctgttccaaaatgtctgaaggtgttgtaacaaccaaaaatgtatcaagatgttgccaaatgtcccttgggaaacaaaatcgctactggttgagaattctgttctattgtttatttaagacgctaatattagggaagtttgggtgaagggtacatgaaaaccctactgtttttgtaactgcccatgtctaaaattatctttataaaaagtatctttttaatattatgctaatacttgtaaaaaatgttctattaaaacttaaactttttttgttttttgagatgaagtctcgctctgtcacctacgctggagtgcagtggtgcgatctcggctcactgcaacctccgccttccaggttcaagtaattctcctgtctcaacctcccgagtagctggaattaaaggtgcccactatcacgcctggctaatttttgtatttttagtaaagacagggtttcaccatgttggccaggctggtctcgaactcctgacttcaggttatccacccacctcggccttccaaagtgctgggattacaggtgtgagccaccgagcctggcttgagtgctttatgtggattaatttatttaatgttcacatcaaacagaaggatgtaattgcttatccccgctttgcagattgggaaaccgaagtacagagtaacgttaagtaatcagttcaaggtcgcatagcaagtgtcagtcccaggctttaaacccaagtctggctccagacttctcttagccttctactgcatcctgcgtctaatttgttgtgggcaggtttccctatgagttcacttcactgagcctcattctttttaacggtcacatagggttctatgctaggtgaactgtagttgcaccctttgctgtcagtgtcttcaactcccatatctacccacagttccccaactctgggtccccaaatacctcccctgtgtcccacgggctctgcagactctattttaagttggctaaaactgagcccatctcctcccacctgcacctcctccgtgttcttatatgaatgtcaatgtcggcacccactggtcacccaggatggaggaaggagccagactgcgagggaataaaggaagtaagcaggttccaaaggacgtggaaggcctttgggtccagagcctagggaaaagttgagccctgcaaaaaataaaagaggctgggttagctttggacatctgtgggaaacaggtgggaagagggaggatggaggagccaaatttaagcatcgggtacttaggtgctcatctccaaggcaactgcattgctatggcaacagcagaggcagcaccgccccgcccaagtacctcctggaccgccgttacctctgcaccagccagactcttccgcatgggagggctgggggcttgcggacctccgggaacagctcaggcaggcgggagttgctgcggcattagccaatccataagctctatcccgcctgggtgggctctgcggcgacgcgcgccaagaaggggtcgggagttttttggcgctcgctaaagggcgttgagaagggcgggcccgcgtcgcttcggaccactcaggagccgaggagagcgagtgggcggggccaggctgaggctggccgggggcggggcgcagggataacgtgggagggtccgcccagcctttcttctacccaatagaaggccagaaagccaatccggaccgttagggagcccaatgggcgtcgccgccaggccccgttgcagagcgcgtctagccaataggcagcggcggcgggcgggcgcgggcgacaggcggcgcagctgaggcggagcaggcgctgcggcaggagggaagatggcggacgaggagaagctgccgcccggctgggagaagcgcatgagccgcagctcag

SEQ ID NO: 2 - DNA, 397 bp, Homo sapiens
ccctcgctggcgtccctgcaccctgggagcgcgagcggcgcgcgggcggggaagcgcggcccagacccccgggtccgcccggagcagctgcgctgtcggggccaggccgggctcccagtggattcgcgggcacagacgcccaggaccgcgcttcccacgtggcggagggactggggacccgggcacccgtcctgccccttcaccttccagctccgcctcctccgcgcggaccccgccccgtcccgacccctcccgggtccccggcccagccccctccgggccctcccagcccctccccttcctttccgcggccccgccctctcctcgcggcgcgagtttcaggcagcgctgcgtcctgctgcgcacgtgggaagccctggccccggccacccccgcg

SEQ ID NO: 3 - DNA, 245 bp, Homo sapiens
ccccacgtggcggagggactggggacccgggcacccgtcctgccccttcaccttccagctccgcctcctccgcgcggaccccgccccgtcccgacccctcccgggtccccggcccagccccctccgggccctcccagcccctccccttcctttccgcggccccgccctctcctcgcggcgcgagtttcaggcagcgctgcgtcctgctgcgcacgtgggaagccctggccccggccacccccgcg

SEQ ID NO: 4 - DNA, 226 bp, Homo sapiens
cgtggcggagggactggggacccgggcacccgtcctgccccttcaccttccagctccgcctcctccgcgcggaccccgccccgtcccgacccctcccgggtccccggcccagccccctccgggccctcccagcccctccccttcctttccgcggccccgccctctcctcgcggcgcgagtttcaggcagcgctgcgtcctgctgcgcacgtgggaagccctggccc

SEQ ID NO: 5 - DNA, 264 bp, Homo sapiens
catccggacaaagcctgcgcgcgccccgccccgccattggccgtaccgccccgcgccgccgccccatctcgcccctcgccgccgggtccggcgcgttaaagccaataggaaccgccgccgttgttcccgtcacggccggggcagccaattgtggcggcgctcggcggctcgtggctctttcgcggcaaaaaggatttggcgcgtaaaagtggccgggactttgcaggcagcggcggccgggggcggagcgggatcgagccctcg

SEQ ID NO: 6 - protein, 19 aa, Foot-and-mouth disease virus
LLNFDLLKLAGDVESNPGP

SEQ ID NO: 7 - protein, 19 aa, Foot-and-mouth disease virus
TLNFDLLKLAGDVESNPGP

SEQ ID NO: 8 - protein, 14 aa, Foot-and-mouth disease virus
LLKLAGDVESNPGP

SEQ ID NO: 9 - protein, 17 aa, Foot-and-mouth disease virus
NFDLLKLAGDVESNPGP

SEQ ID NO: 10 - protein, 20 aa, Foot-and-mouth disease virus
QLLNFDLLKLAGDVESNPGP

SEQ ID NO: 11 - protein, 24 aa, Foot-and-mouth disease virus
APVKQTLNFDLLKLAGDVESNPGP

SEQ ID NO: 12 - protein, 58 aa, Foot-and-mouth disease virus
VTELLYRMKRAETYCPRPLLAIHPTEARHKQKIVAPVKQTLNFDLLKLAGDVESNPGP

SEQ ID NO: 13 - protein, 40 aa, Foot-and-mouth disease virus
LLAIHPTEARHKQKIVAPVKQTLNFDLLKLAGDVESNPGP

SEQ ID NO: 14 - protein, 33 aa, Foot-and-mouth disease virus
EARHKQKIVAPVKQTLNFDLLKLAGDVESNPGP

SEQ ID NO: 15 - protein, 19 aa, Foot-and-mouth disease virus
NFDLLKLAGDVESNPGPFF

SEQ ID NO: 16 - protein, 25 aa, Encephalomyocarditis virus
GIFNAHYAGYFADLLIHDIETNPGP

SEQ ID NO: 17 - protein, 25 aa, Encephalomyocarditis virus
RIFNAHYAGYFADLLIHDIETNPGP

SEQ ID NO: 18 - protein, 25 aa, Encephalomyocarditis virus
HVFETHYAGYFADLLIHDVETNPGP

SEQ ID NO: 19 - protein, 25 aa, Theiler′s encephalomyelitis virus
KAVRGYHADYYKQRLIHDVEMNPGP

SEQ ID NO: 20 - protein, 25 aa, Theiler′s encephalomyelitis virus
RAVRAYHADYYKQRLIHDVEMNPGP

SEQ ID NO: 21 - protein, 25 aa, Theiler′s encephalomyelitis virus
KAVRGYHADYYRQRLIHDVETNPGP

SEQ ID NO: 22 - protein, 19 aa, Foot-and-mouth disease virus
LTNFDLLKLAGDVESNPGP

SEQ ID NO: 23 - protein, 19 aa, Foot-and-mouth disease virus
LLNFDLLKLAGDMESNPGP

SEQ ID NO: 24 - protein, 19 aa, Foot-and-mouth disease virus
MCNFDLLKLAGDVESNPGP

SEQ ID NO: 25 - protein, 19 aa, Equine rhinitis A virus
CTNYALLKLAGDVESNPGP

SEQ ID NO: 26 - protein, 20 aa, Equine rhinitis A virus
GATNFSLLKLAGDVELNPGP

SEQ ID NO: 27 - protein, 22 aa, Porcine teschovirus
GPGATNFSLLKQAGDVEENPGP

SEQ ID NO: 28 - protein, 20 aa, Drosophila C virus
EAARQMLLLLSGDVETNPGP

SEQ ID NO: 29 - protein, 20 aa, Cricket paralysis virus
FLRKRTQLLMSGDVESNPGP

SEQ ID NO: 30 - protein, 20 aa, Acute bee paralysis virus
GSWTDILLLLSGDVETNPGP

SEQ ID NO: 31 - protein, 20 aa, Thosea asigna virus
RAEGRGSLLTCGDVEENPGP

SEQ ID NO: 32 - protein, 20 aa, Infectious flacherie virus
TRAEIEDELIRAGIESNPGP

SEQ ID NO: 33 - protein, 20 aa, Bovine rotavirus
SKFQIDRILISGDIELNPGP

SEQ ID NO: 34 - protein, 20 aa, Porcine rotavirus
AKFQIDKILISGDVELNPGP

SEQ ID NO: 35 - protein, 20 aa, Human rotavirus
SKFQIDKILISGDIELNPGP

SEQ ID NO: 36 - protein, 20 aa, Trypanosoma brucei
SSIIRTKMLVSGDVEENPGP

SEQ ID NO: 37 - protein, 20 aa, Trypanosoma cruzi
CDAQRQKLLLSGDIEQNPGP

SEQ ID NO: 38 - protein, 5 aa, Artificial Sequence
  Description of Artificial Sequence Synthetic peptide
RXKRR

SEQ ID NO: 39 - protein, 4 aa, Artificial Sequence
  Description of Artificial Sequence Synthetic peptide
RAKR

SEQ ID NO: 40 - protein, 5 aa, Artificial Sequence
  Description of Artificial Sequence Synthetic peptide
IEDGR

SEQ ID NO: 41 - protein, 10 aa, Artificial Sequence
  Description of Artificial Sequence Synthetic peptide
LAGFATVAQA

SEQ ID NO: 42 - protein, 6 aa, Artificial Sequence
  Description of Artificial Sequence Synthetic peptide
LVPRGS

SEQ ID NO: 43 - protein, 5 aa, Artificial Sequence
  Description of Artificial Sequence Synthetic peptide
XXGGX

SEQ ID NO: 44 - protein, 5 aa, Artificial Sequence
  Description of Artificial Sequence Synthetic peptide
XXGXG

SEQ ID NO: 45 - DNA, 303 bp, Homo sapiens
accggttttttggcgctcgctaaagggcgttgagaagggcgggcccgcgtcgcttcggaccactcaggagccgaggagagcgagtgggcggggccaggctgaggctggccgggggcggggcgcagggataacgtgggagggtccgcccagcctttcttctacccaatagaaggccagaaagccaatccggaccgttagggagcccattgggcgtcgccgccaggccccgttgcagagcgcgtctagccaataggcagcggcggcgggcgggcgcgggcgacaggcggcgcagctgagaccggt

SEQ ID NO: 46 - DNA, 380 bp, Homo sapiens
accggtcggcattagccaatccataagctctatcccgcctgggtgggctctgcggcgacgcgcgccaagaaggggtcgggagttttttggcgctcgctaaagggcgttgagaagggcgggcccgcgtcgcttcggaccactcaggagccgaggagagcgagtgggcggggccaggctgaggctggccgggggcggggcgcagggataacgtgggagggtccgcccagcctttcttctacccaatagaaggccagaaagccaatccggaccgttagggagcccattgggcgtcgccgccaggccccgttgcagagcgcgtctagccaataggcagcggcggcgggcgggcgcgggcgacaggcggcgcagctgagaccggt

SEQ ID NO: 47 - DNA, 19 bp, Artificial Sequence
  Description of Artificial Sequence Synthetic primer
tcgtcttcaagaattctca

SEQ ID NO: 48 - DNA, 21 bp, Artificial Sequence
  Description of Artificial Sequence Synthetic primer
gtatataatgcggccgtgggc

SEQ ID NO: 49 - DNA, 21 bp, Artificial Sequence
  Description of Artificial Sequence Synthetic primer
gcccacggccgcattatatac

SEQ ID NO: 50 - DNA, 21 bp, Artificial Sequence
  Description of Artificial Sequence Synthetic primer
ccagaaaatccagcaggtacc

SEQ ID NO: 51 - DNA, 36 bp, Artificial Sequence
  Description of Artificial Sequence Synthetic primer
agctgaattctcgagttggagccactatcgactacg

SEQ ID NO: 52 - DNA, 30 bp, Artificial Sequence
  Description of Artificial Sequence Synthetic primer
agctaccggtcacgtaaacggtcaaagtcc

SEQ ID NO: 53 - DNA, 34 bp, Artificial Sequence
  Description of Artificial Sequence Synthetic primer
ataccggtggtaccatccggacaaagcctgcgcg

SEQ ID NO: 54 - DNA, 29 bp, Artificial Sequence
  Description of Artificial Sequence Synthetic primer
agaccggtcgagggctcgatcccgctccg

SEQ ID NO: 55 - DNA, 34 bp, Artificial Sequence
  Description of Artificial Sequence Synthetic primer
atgcagcgtcgacgctccagtaaagcagactcta

SEQ ID NO: 56 - DNA, 34 bp, Artificial Sequence
  Description of Artificial Sequence Synthetic primer
catgatcgtcgactggacctgggttgctctcaac

SEQ ID NO: 57 - DNA, 21 bp, Artificial Sequence
  Description of Artificial Sequence Synthetic primer
tgtgtctagagaatgcaatag

SEQ ID NO: 58 - DNA, 33 bp, Artificial Sequence
  Description of Artificial Sequence Synthetic primer
gatatatgtcgactggcctggggcgtttacagc

SEQ ID NO: 59 - DNA, 35 bp, Artificial Sequence
  Description of Artificial Sequence Synthetic primer
gacatgcgtcgacatggagcgaagaaacccatctg

SEQ ID NO: 60 - DNA, 23 bp, Artificial Sequence
  Description of Artificial Sequence Synthetic primer
ccatagaagcttacaccgtgtag

SEQ ID NO: 61 - DNA, 42 bp, Artificial Sequence
  Description of Artificial Sequence Synthetic primer
ccgctcgagcggttaattaaccacctcaatctgtatcttcat

SEQ ID NO: 62 - DNA, 42 bp, Artificial Sequence
  Description of Artificial Sequence Synthetic primer
ggttaattaaccgctcgagcggactgaaatgtgtgggcgtgg

SEQ ID NO: 63 - DNA, 20 bp, Artificial Sequence
  Description of Artificial Sequence Synthetic primer
tgagacgcccgacatcacct

SEQ ID NO: 64 - DNA, 19 bp, Artificial Sequence
  Description of Artificial Sequence Synthetic primer
tggctgcagcggctgaagc

SEQ ID NO: 65 - DNA, 32 bp, Artificial Sequence
  Description of Artificial Sequence Synthetic primer
atgcgaccggtcggcattagccaatccataag

SEQ ID NO: 66 - DNA, 32 bp, Artificial Sequence
  Description of Artificial Sequence Synthetic primer
atgcgaccggtaaggggtcgggagttttttgg

SEQ ID NO: 67 - DNA, 32 bp, Artificial Sequence
  Description of Artificial Sequence Synthetic primer
atgcgaccggtctcagctgcgccgcctgtcgc

SEQ ID NO: 68 - DNA, 9703 bp, Artificial Sequence
  Description of Artificial Sequence Synthetic CP1521 sequence
gatccggggcccccatttcccctcccttccagctctctgccccttttggattgaagccaatatgataatgagggggtggagtttgtgacgtggcgcggggcgtgggaacggggcgggtgacgtagtagtgtggcggaagtgtgatgttgcaagtgtggcggaacacatgtaagcgacggatgtggcaaaagtgacgtttttggtgtgcgccggtgtacacaggaagtgacaattttcgcgcggttttaggcggatgttgtagtaaatttgggcgtaaccgagtaagatttggccattttcgcgggaaaactgaataagaggaagtgaaatctgaataattttgtgttactcatagcgcgtaatatttgtctagggccgcggggactttgaccgtttacgtgaccggttttttggcgctcgctaaagggcgttgagaagggcgggcccgcgtcgcttcggaccactcaggagccgaggagagcgagtgggcggggccaggctgaggctggccgggggcggggcgcagggataacgtgggagggtccgcccagcctttcttctacccaatagaaggccagaaagccaatccggaccgttagggagcccaatgggcgtcgccgccaggccccgttgcagagcgcgtctagccaataggcagcggcggcgggcgggcgcgggcgacaggcggcgcagctgagaccggtgactgaaaatgagacatattatctgccacggaggtgttattaccgaagaaatggccgccagtcttttggaccagctgatcgaagaggtactggctgataatcttccacctcctagccattttgaaccacctacccttcacgaactgtatgatttagacgtgacggcccccgaagatcccaacgaggaggcggtttcgcagatttttcccgactctgtaatgttggcggtgcaggaagggattgacttactcacttttccgccggcgcccggttctccggaggcgcctcacctttcccggcagcccgagcagccggagcagagagccttgggtccggtttctatgccaaaccttgtaccggaggtgatcgatcttacctgccacgaggctggctttccacccagtgacgacgaggatgaagagggtgaggagtttgtgttagattatgtggagcaccccgggcacggttgcaggtcttgtcattatcaccggaggaatacgggggacccagatattatgtgttcgctttgctatatgaggacctgtggcatgtttgtctacagtaagtgaaaattatgggcagtgggtgatagagtggtgggtttggtgtggtaatttttttttaatttttacagttttgtggtttaaagaattttgtattgtgatttttttaaaaggtcctgtgtctgaacctgagcctgagcccgagccagaaccggagcctgcaagacctacccgccgtcctaaaatggcgcctgctatcctgagacgcccgacgtcacctgtgtctagagaatgcaatagtagtacggatagctgtgactccggtccttctaacacacctcctgagatacacccggtggtcccgctgtgccccattaaaccagttgccgtgagagttggtgggcgtcgccaggctgtggaatgtatcgaggacttgcttaacgagcctgggcaacctttggacttgagctgtaaacgccccaggccataaggtgtaaacctgtgattgcgtgtgtggttaacgcctttgtttgctgaatgagttgatgtaagtttaataaagggtgagataatgtttaacttgcatggcgtgttaaatggggcggggcttaaagggtatataatgcgccgtgggctaatcttggttacatctgacctcatggaggcttgggagtgtttggaagatttttctgctgtgcgtaacttgctggaacagagctctaacagtacctcttggttttggaggtttctgtggggctcatcccaggcaaagttagtctgcagaattaaggaggattacaagtgggaatttgaagagcttttgaaatcctgtggtgagctgtttgattctttgaatctgggtcaccaggcgcttttccaagagaaggtcatcaagactttggatttttccacaccggggcgcgctgcggctgctgttgcttttttgagttttataaaggataaatggagcgaagaaacccatctgagcggggggtacctgctggattttctggccatgcatctgtggagagcggttgtgagacacaagaatcgcctgctactgttgtcttccgtccgcccggcgataataccgacggaggagcagcagcagcagcaggaggaagccaggcggcggcggcaggagcagagcccatggaacccgagagccggcctggaccctcgggaatgaatgttgtacaggtggctgaactgtatccagaactgagacgcattttgacaattacagaggatgggcaggggctaaagggggtaaagagggagcggggggcttgtgaggctacagaggaggctaggaatctagcttttagcttaatgaccagacaccgtcctgagtgtattacttttcaacagatcaaggataattgcgctaatgagcttgatctgctggcgcagaagtattccatagagcagctgaccacttactggctgcagccaggggatgattttgaggaggctattagggtatatgcaaaggtggcacttaggccagattgcaagtacaagatcagcaaacttgtaaatatcaggaattgttgctacatttctgggaacggggccgaggtggagatagatacggaggatagggtggcctttagatgtagcatgataaatatgtggccgggggtgcttggcatggacggggtggttattatgaatgtaaggtttactggccccaattttagcggtacggttttcctggccaataccaaccttatcctacacggtgtaagcttctatgggtttaacaatacctgtgtggaagcctggaccgatgtaagggttcggggctgtgccttttactgctgctggaagggggtggtgtgtcgccccaaaagcagggcttcaattaagaaatgcctctttgaaaggtgtaccttgggtatcctgtctgagggtaactccagggtgcgccacaatgtggcctccgactgtggttgcttcatgctagtgaaaagcgtggctgtgattaagcataacatggtatgtggcaactgcgaggacagggcctctcagatgctgacctgctcggacggcaactgtcacctgctgaagaccattcacgtagccagccactctcgcaaggcctggccagtgtttgagcataacatactgacccgctgttccttgcatttgggtaacaggaggggggtgttcctaccttaccaatgcaatttgagtcacactaagatattgcttgagcccgagagcatgtccaaggtgaacctgaacggggtgtttgacatgaccatgaagatctggaaggtgctgaggtacgatgagacccgcaccaggtgcagaccctgcgagtgtggcggtaaacatattaggaaccagcctgtgatgctggatgtgaccgaggagctgaggcccgatcacttggtgctggcctgcacccgcgctgagtttggctctagcgatgaagatacagattgaggtactgaaatgtgtgggcgtggcttaagggtgggaaagaatatataaggtgggggtcttatgtagttttgtatctgttttgcagcagccgccgccgccatgagcaccaactcgtttgatggaagcattgtgagctcatatttgacaacgcgcatgcccccatgggccggggtgcgtcagaatgtgatgggctccagcattgatggtcgccccgtcctgcccgcaaactctactaccttgacctacgagaccgtgtctggaacgccgttggagactgcagcctccgccgccgcttcagccgctgcagccaccgcccgcgggattgtgactgactttgctttcctgagcccgcttgcaagcagtgcagcttcccgttcatccgcccgcgatgacaagttgacggctcttttggcacaattggattctttgacccgggaacttaatgtcgtttctcagcagctgttggatctgcgccagcaggtttctgccctgaaggcttcctcccctcccaatgcggtttaaaacataaataaaaaaccagactctgtttggatttggatcaagcaagtgtcttgctgtctttatttaggggttttgcgcgcgcggtaggcccgggaccagcggtctcggtcgttgagggtcctgtgtattttttccaggacgtggtaaaggtgactctggatgttcagatacatgggcataagcccgtctctggggtggaggtagcaccactgcagagcttcatgctgcggggtggtgttgtagatgatccagtcgtagcaggagcgctgggcgtggtgcctaaaaatgtctttcagtagcaagctgattgccaggggcaggcccttggtgtaagtgtttacaaagcggttaagctgggatgggtgcatacgtggggatatgagatgcatcttggactgtatttttaggttggctatgttcccagccatatccctccggggattcatgttgtgcagaaccaccagcacagtgtatccggtgcacttgggaaatttgtcatgtagcttagaaggaaatgcgtggaagaacttggagacgcccttgtgacctccaagattttccatgcattcgtccataatgatggcaatgggcccacgggcggcggcctgggcgaagatatttctgggatcactaacgtcatagttgtgttccaggatgagatcgtcataggccatttttacaaagcgcgggcggagggtgccagactgcggtataatggttccatccggcccaggggcgtagttaccctcacagatttgcatttcccacgctttgagttcagatggggggatcatgtctacctgcggggcgatgaagaaaacggtttccggggtaggggagatcagctgggaagaaagcaggttcctgagcagctgcgacttaccgcagccggtgggcccgtaaatcacacctattaccgggtgcaactggtagttaagagagctgcagctgccgtcatccctgagcaggggggccacttcgttaagcatgtccctgactcgcatgttttccctgaccaaatccgccagaaggcgctcgccgcccagcgatagcagttcttgcaaggaagcaaagtttttcaacggtttgagaccgtccgccgtaggcatgcttttgagcgtttgaccaagcagttccaggcggtcccacagctcggtcacctgctctacggcatctcgatccagcatatctcctcgtttcgcgggttggggcggctttcgctgtacggcagtagtcggtgctcgtccagacgggccagggtcatgtctttccacgggcgcagggtcctcgtcagcgtagtctgggtcacggtgaaggggtgcgctccgggctgcgcgctggccagggtgcgcttgaggctggtcctgctggtgctgaagcgctgccggtcttcgccctgcgcgtcggccaggtagcatttgaccatggtgtcatagtccagcccctccgcggcgtggcccttggcgcgcagcttgcccttggaggaggcgccgcacgaggggcagtgcagacttttgagggcgtagagcttgggcgcgagaaataccgattccggggagtaggcatccgcgccgcaggccccgcagacggtctcgcattccacgagccaggtgagctctggccgttcggggtcaaaaaccaggtttcccccatgctttttgatgcgtttcttacctctggtttccatgagccggtgtccacgctcggtgacgaaaaggctgtccgtgtccccgtatacagacttgagaggcctgtcctcgaccgatgcccttgagagccttcaacccagtcagctccttccggtgggcgcggggcatgactatcgtcgccgcacttatgactgtcttctttatcatgcaactcgtaggacaggtgccggcagcgctctgggtcattttcggcgaggaccgctttcgctggagcgcgacgatgatcggcctgtcgcttgcggtattcggaatcttgcacgccctcgctcaagccttcgtcactggtcccgccaccaaacgtttcggcgagaagcaggccattatcgccggcatggcgacgcgctgggctacgtcttgctggcgttcgcgacgcgaggctggatggccttccccattatgattcttctcgcttccggcggcatcgggatgcccgcgttgcaggccatgctgtccaggcaggtagatgacgaccatcagggacagcttcaaggatcgctcgcggctcttaccagcctaacttcgatcactggaccgctgatcgtcacggcgatttatgccgcctcggcgagcacatggaacgggttggcatggattgtaggcgccgccctataccttgtctgcctccccgcgttgcgtcgcggtgcatggagccgggccacctcgacctgaatggaagccggcggcacctcgctaacggattcaccactccaagaattggagccaatcaattcttgcggagaactgtgaatgcgcaaaccaacccttggcagaacatatccatcgcgtccgccatctccagcagccgcacgcggcgcatctcgggcagcgttgggtcctggccacgggtgcgcatgatcgtgctcctgtcgttgaggacccggctaggctggcggggttgccttactggttagcagaatgaatcaccgatacgcgagcgaacgtgaagcgactgctgctgcaaaacgtctgcgacctgagcaacaacatgaatggtcttcggtttccgtgtttcgtaaagtctggaaacgcggaagtcagcgccctgcaccattatgttccggatctgcatcgcaggatgctgctggctaccctgtggaacacctacatctgtattaacgaagcgctggcattgaccctgagtgatttttctctggtcccgccgcatccataccgccagttgtttaccctcacaacgttccagtaaccgggcatgttcatcatcagtaacccgtatcgtgagcatcctctctcgtttcatcggtatcattacccccatgaacagaaatcccccttacacggaggcatcagtgaccaaacaggaaaaaaccgcccttaacatggcccgctttatcagaagccagacattaacgcttctggagaaactcaacgagctggacgcggatgaacaggcagacatctgtgaatcgcttcacgaccacgctgatgagctttaccgcagctgcctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggcgcagccatgacccagtcacgtagcgatagcggagtgtatactggcttaactatgcggcatcagagcagattgtactgagagtgcaccatatgcggtgtgaaataccgcacagatgcgtaaggagaaaataccgcatcaggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctgcaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaacacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtcttcaagaattctcgagttggagccactatcgactacgcgatcatggcgaccacacccgtcctgtg

SEQ ID NO: 69 - DNA, 9780 bp, Artificial Sequence
  Description of Artificial Sequence Synthetic CP1522 sequence
gatccggggcccccatttcccctcccttccagctctctgccccttttggattgaagccaatatgataatgagggggtggagtttgtgacgtggcgcggggcgtgggaacggggcgggtgacgtagtagtgtggcggaagtgtgatgttgcaagtgtggcggaacacatgtaagcgacggatgtggcaaaagtgacgtttttggtgtgcgccggtgtacacaggaagtgacaattttcgcgcggttttaggcggatgttgtagtaaatttgggcgtaaccgagtaagatttggccattttcgcgggaaaactgaataagaggaagtgaaatctgaataattttgtgttactcatagcgcgtaatatttgtctagggccgcggggactttgaccgtttacgtgaccggtcggcattagccaatccataagctctatcccgcctgggtgggctctgcggcgacgcgcgccaagaaggggtcgggagttttttggcgctcgctaaagggcgttgagaagggcgggcccgcgtcgcttcggaccactcaggagccgaggagagcgagtgggcggggccaggctgaggctggccgggggcggggcgcagggataacgtgggagggtccgcccagcctttcttctacccaatagaaggccagaaagccaatccggaccgttagggagcccaatgggcgtcgccgccaggccccgttgcagagcgcgtctagccaataggcagcggcggcgggcgggcgcgggcgacaggcggcgcagctgagaccggtgactgaaaatgagacatattatctgccacggaggtgttattaccgaagaaatggccgccagtcttttggaccagctgatcgaagaggtactggctgataatcttccacctcctagccattttgaaccacctacccttcacgaactgtatgatttagacgtgacggcccccgaagatcccaacgaggaggcggtttcgcagatttttcccgactctgtaatgttggcggtgcaggaagggattgacttactcacttttccgccggcgcccggttctccggaggcgcctcacctttcccggcagcccgagcagccggagcagagagccttgggtccggtttctatgccaaaccttgtaccggaggtgatcgatcttacctgccacgaggctggctttccacccagtgacgacgaggatgaagagggtgaggagtttgtgttagattatgtggagcaccccgggcacggttgcaggtcttgtcattatcaccggaggaatacgggggacccagatattatgtgttcgctttgctatatgaggacctgtggcatgtttgtctacagtaagtgaaaattatgggcagtgggtgatagagtggtgggtttggtgtggtaatttttttttaatttttacagttttgtggtttaaagaattttgtattgtgatttttttaaaaggtcctgtgtctgaacctgagcctgagcccgagccagaaccggagcctgcaagacctacccgccgtcctaaaatggcgcctgctatcctgagacgcccgacgtcacctgtgtctagagaatgcaatagtagtacggatagctgtgactccggtccttctaacacacctcctgagatacacccggtggtcccgctgtgccccattaaaccagttgccgtgagagttggtgggcgtcgccaggctgtggaatgtatcgaggacttgcttaacgagcctgggcaacctttggacttgagctgtaaacgccccaggccataaggtgtaaacctgtgattgcgtgtgtggttaacgcctttgtttgctgaatgagttgatgtaagtttaataaagggtgagataatgtttaacttgcatggcgtgttaaatggggcggggcttaaagggtatataatgcgccgtgggctaatcttggttacatctgacctcatggaggcttgggagtgtttggaagatttttctgctgtgcgtaacttgctggaacagagctctaacagtacctcttggttttggaggtttctgtggggctcatcccaggcaaagttagtctgcagaattaaggaggattacaagtgggaatttgaagagcttttgaaatcctgtggtgagctgtttgattctttgaatctgggtcaccaggcgcttttccaagagaaggtcatcaagactttggatttttccacaccggggcgcgctgcggctgctgttgcttttttgagttttataaaggataaatggagcgaagaaacccatctgagcggggggtacctgctggattttctggccatgcatctgtggagagcggttgtgagacacaagaatcgcctgctactgttgtcttccgtccgcccggcgataataccgacggaggagcagcagcagcagcaggaggaagccaggcggcggcggcaggagcagagcccatggaacccgagagccggcctggaccctcgggaatgaatgttgtacaggtggctgaactgtatccagaactgagacgcattttgacaattacagaggatgggcaggggctaaagggggtaaagagggagcggggggcttgtgaggctacagaggaggctaggaatctagcttttagcttaatgaccagacaccgtcctgagtgtattacttttcaacagatcaaggataattgcgctaatgagcttgatctgctggcgcagaagtattccatagagcagctgaccacttactggctgcagccaggggatgattttgaggaggctattagggtatatgcaaaggtggcacttaggccagattgcaagtacaagatcagcaaacttgtaaatatcaggaattgttgctacatttctgggaacggggccgaggtggagatagatacggaggatagggtggcctttagatgtagcatgataaatatgtggccgggggtgcttggcatggacggggtggttattatgaatgtaaggtttactggccccaattttagcggtacggttttcctggccaataccaaccttatcctacacggtgtaagcttctatgggtttaacaatacctgtgtggaagcctggaccgatgtaagggttcggggctgtgccttttactgctgctggaagggggtggtgtgtcgccccaaaagcagggcttcaattaagaaatgcctctttgaaaggtgtaccttgggtatcctgtctgagggtaactccagggtgcgccacaatgtggcctccgactgtggttgcttcatgctagtgaaaagcgtggctgtgattaagcataacatggtatgtggcaactgcgaggacagggcctctcagatgctgacctgctcggacggcaactgtcacctgctgaagaccattcacgtagccagccactctcgcaaggcctggccagtgtttgagcataacatactgacccgctgttccttgcatttgggtaacaggaggggggtgttcctaccttaccaatgcaatttgagtcacactaagatattgcttgagcccgagagcatgtccaaggtgaacctgaacggggtgtttgacatgaccatgaagatctggaaggtgctgaggtacgatgagacccgcaccaggtgcagaccctgcgagtgtggcggtaaacatattaggaaccagcctgtgatgctggatgtgaccgaggagctgaggcccgatcacttggtgctggcctgcacccgcgctgagtttggctctagcgatgaagatacagattgaggtactgaaatgtgtgggcgtggcttaagggtgggaaagaatatataaggtgggggtcttatgtagttttgtatctgttttgcagcagccgccgccgccatgagcaccaactcgtttgatggaagcattgtgagctcatatttgacaacgcgcatgcccccatgggccggggtgcgtcagaatgtgatgggctccagcattgatggtcgccccgtcctgcccgcaaactctactaccttgacctacgagaccgtgtctggaacgccgttggagactgcagcctccgccgccgcttcagccgctgcagccaccgcccgcgggattgtgactgactttgctttcctgagcccgcttgcaagcagtgcagcttcccgttcatccgcccgcgatgacaagttgacggctcttttggcacaattggattctttgacccgggaacttaatgtcgtttctcagcagctgttggatctgcgccagcaggtttctgccctgaaggcttcctcccctcccaatgcggtttaaaacataaataaaaaaccagactctgtttggatttggatcaagcaagtgtcttgctgtctttatttaggggttttgcgcgcgcggtaggcccgggaccagcggtctcggtcgttgagggtcctgtgtattttttccaggacgtggtaaaggtgactctggatgttcagatacatgggcataagcccgtctctggggtggaggtagcaccactgcagagcttcatgctgcggggtggtgttgtagatgatccagtcgtagcaggagcgctgggcgtggtgcctaaaaatgtctttcagtagcaagctgattgccaggggcaggcccttggtgtaagtgtttacaaagcggttaagctgggatgggtgcatacgtggggatatgagatgcatcttggactgtatttttaggttggctatgttcccagccatatccctccggggattcatgttgtgcagaaccaccagcacagtgtatccggtgcacttgggaaatttgtcatgtagcttagaaggaaatgcgtggaagaacttggagacgcccttgtgacctccaagattttccatgcattcgtccataatgatggcaatgggcccacgggcggcggcctgggcgaagatatttctgggatcactaacgtcatagttgtgttccaggatgagatcgtcataggccatttttacaaagcgcgggcggagggtgccagactgcggtataatggttccatccggcccaggggcgtagttaccctcacagatttgcatttcccacgctttgagttcagatggggggatcatgtctacctgcggggcgatgaagaaaacggtttccggggtaggggagatcagctgggaagaaagcaggttcctgagcagctgcgacttaccgcagccggtgggcccgtaaatcacacctattaccgggtgcaactggtagttaagagagctgcagctgccgtcatccctgagcaggggggccacttcgttaagcatgtccctgactcgcatgttttccctgaccaaatccgccagaaggcgctcgccgcccagcgatagcagttcttgcaaggaagcaaagtttttcaacggtttgagaccgtccgccgtaggcatgcttttgagcgtttgaccaagcagttccaggcggtcccacagctcggtcacctgctctacggcatctcgatccagcatatctcctcgtttcgcgggttggggcggctttcgctgtacggcagtagtcggtgctcgtccagacgggccagggtcatgtctttccacgggcgcagggtcctcgtcagcgtagtctgggtcacggtgaaggggtgcgctccgggctgcgcgctggccagggtgcgcttgaggctggtcctgctggtgctgaagcgctgccggtcttcgccctgcgcgtcggccaggtagcatttgaccatggtgtcatagtccagcccctccgcggcgtggcccttggcgcgcagcttgcccttggaggaggcgccgcacgaggggcagtgcagacttttgagggcgtagagcttgggcgcgagaaataccgattccggggagtaggcatccgcgccgcaggccccgcagacggtctcgcattccacgagccaggtgagctctggccgttcggggtcaaaaaccaggtttcccccatgctttttgatgcgtttcttacctctggtttccatgagccggtgtccacgctcggtgacgaaaaggctgtccgtgtccccgtatacagacttgagaggcctgtcctcgaccgatgcccttgagagccttcaacccagtcagctccttccggtgggcgcggggcatgactatcgtcgccgcacttatgactgtcttctttatcatgcaactcgtaggacaggtgccggcagcgctctgggtcattttcggcgaggaccgctttcgctggagcgcgacgatgatcggcctgtcgcttgcggtattcggaatcttgcacgccctcgctcaagccttcgtcactggtcccgccaccaaacgtttcggcgagaagcaggccattatcgccggcatggcgacgcgctgggctacgtcttgctggcgttcgcgacgcgaggctggatggccttccccattatgattcttctcgcttccggcggcatcgggatgcccgcgttgcaggccatgctgtccaggcaggtagatgacgaccatcagggacagcttcaaggatcgctcgcggctcttaccagcctaacttcgatcactggaccgctgatcgtcacggcgatttatgccgcctcggcgagcacatggaacgggttggcatggattgtaggcgccgccctataccttgtctgcctccccgcgttgcgtcgcggtgcatggagccgggccacctcgacctgaatggaagccggcggcacctcgctaacggattcaccactccaagaattggagccaatcaattcttgcggagaactgtgaatgcgcaaaccaacccttggcagaacatatccatcgcgtccgccatctccagcagccgcacgcggcgcatctcgggcagcgttgggtcctggccacgggtgcgcatgatcgtgctcctgtcgttgaggacccggctaggctggcggggttgccttactggttagcagaatgaatcaccgatacgcgagcgaacgtgaagcgactgctgctgcaaaacgtctgcgacctgagcaacaacatgaatggtcttcggtttccgtgtttcgtaaagtctggaaacgcggaagtcagcgccctgcaccattatgttccggatctgcatcgcaggatgctgctggctaccctgtggaacacctacatctgtattaacgaagcgctggcattgaccctgagtgatttttctctggtcccgccgcatccataccgccagttgtttaccctcacaacgttccagtaaccgggcatgttcatcatcagtaacccgtatcgtgagcatcctctctcgtttcatcggtatcattacccccatgaacagaaatcccccttacacggaggcatcagtgaccaaacaggaaaaaaccgcccttaacatggcccgctttatcagaagccagacattaacgcttctggagaaactcaacgagctggacgcggatgaacaggcagacatctgtgaatcgcttcacgaccacgctgatgagctttaccgcagctgcctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggcgcagccatgacccagtcacgtagcgatagcggagtgtatactggcttaactatgcggcatcagagcagattgtactgagagtgcaccatatgcggtgtgaaataccgcacagatgcgtaaggagaaaataccgcatcaggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctgcaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaacacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtcttcaagaattctcgagttggagccactatcgactacgcgatcatggcgaccacacccgtcctgtg

SEQ ID NO: 70 - protein, 5 aa, Artificial Sequence
  Description of Artificial Sequence Synthetic peptide
RXRKR

SEQ ID NO: 71 - protein, 4 aa, Artificial Sequence
  Description of Artificial Sequence Synthetic peptide
RKKR

SEQ ID NO: 72 - protein, 4 aa, Artificial Sequence
  Description of Artificial Sequence Synthetic peptide
RKRR

SEQ ID NO: 73 - protein, 4 aa, Artificial Sequence
  Description of Artificial Sequence Synthetic peptide
RRRR

(19) United States
(12) Patent Application Publication
(10) Pub. No.: US 2006/0228336 A1
(43) Pub. Date: Oct. 12, 2006

(54) Human prolyl isomerase 1 (PIN 1) promoter and uses thereof

(76) Inventor: Derek Ko, San Francisco, CA (US)

Correspondence Address: Patent Group;DLA PIPER RUDNICK GRAY CARY US LLP, 1200 Nineteenth Street, N.W., Washington, DC, 20036-2412, US

(21) Appl. No.: 11/247,272

(22) Filed: Oct. 12, 2005

Related U.S. Application Data
(60) Provisional application No. 60/617,206, filed on Oct. 12, 2004.

(51) Int. Cl. A61K 48/00; C12N 9/90; C12N 15/861

(52) U.S. Cl. 424/93.2; 435/456; 435/233

(57) ABSTRACT

PIN1 transcriptional regulatory sequences (TREs) and vectors comprising the same are provided. These include replication competent vectors and replication incompetent vectors. PIN1 TREs provide for transcriptional regulation dependent upon transcription factors that are specifically active in cancer cells. The PIN1 TREs may be used as a vehicle for introducing new genetic capability, particularly associated with cytotoxicity and for selective expression in cancer cells.

This application claims priority from U.S. Provisional Application Ser. No. 60/617,206 filed Oct. 12, 2004. The entirety of that provisional application is incorporated herein by reference.

### BACKGROUND OF THE INVENTION

1. Field of the Invention

The invention relates to prolyl isomerase (PIN1) regulatory sequences. The invention further relates to vectors and vector compositions comprising PIN1 regulatory sequences and methods for use in therapy of cancer.

2. Background of the Technology

Currently, standard medical treatments for treatment of cancer including chemotherapy, surgery, radiation therapy and cellular therapy, have clear limitations with regard to both efficacy and toxicity. To date, these approaches have met with varying degrees of success dependent upon the type of cancer, general health of the patient and stage of disease at the time of diagnosis. Improved strategies that combine these standard medical treatments with novel approaches may provide a means for enhanced efficacy and decreased toxicity. A major, indeed the overwhelming, obstacle to cancer therapy is the problem of selectivity, that is, the ability to inhibit the multiplication of tumor cells, while leaving unaffected the function of normal cells. Thus, more effective treatment methods and pharmaceutical compositions for therapy and prophylaxis of cancer are needed.

Vector-mediated gene delivery forms the basis of an innovative and potentially powerful disease-fighting tool in which an exogenous nucleotide is provided to a cell by way of a delivery vehicle such as a viral or non-viral vector. This approach holds great potential in treating not only many forms of cancer, but other diseases as well. A number of vectors have been described as both vehicles for gene therapy and as candidate anticancer agents. An adenoviral vector containing the gene for p53 (which is mutated or inactivated in many cancers such as head and neck squamous cell carcinoma) has recently been approved for gene therapy of cancer in China. (New Scientist, 2003). Adenovirus has emerged as a virus that can be engineered with oncotropic properties. See, for example, U.S. Pat. No. 5,747,469; U.S. Pat. No. 5,801,029; U.S. Pat. No. 5,846,945; U.S. Pat. No. 5,747,469; WO 99/59604; WO 98/35554; WO 98/29555; U.S. Pat. Nos. 6,638,762; and 6,676,935. Specific attenuated replication-competent viral vectors have been developed for which selective replication in cancer cells destroys those cells. For example, various cell-specific replication-competent adenovirus vectors, which preferentially replicate (and thus destroy) certain cell types, are described, for example, in WO 95/19434, WO 98/39465, WO 98/39467, WO 98/39466, WO 99/06576, WO 98/39464, WO 00/15820. Improving the delivery of these vectors, both to local-regional and disseminated disease, as well as improving the vectors to promote intratumoral spread is of particular interest.

Prolyl isomerase 1 (PIN1) catalyzes the conversion of proteins containing phosphorylated pSer/Thr-Pro motifs. Overexpression of PIN1 has been shown to positively regulate cyclin D1 via transcriptional activation and posttranslational stabilization. PIN1 has also been found to regulate the degradation and localization of beta-catenin [Ryo et al. (September 2001); Nature Cell Biology pp. 793-801]. Additionally, reports in the literature suggest that PIN1 plays a key role in p53-mediated apoptosis [Zacchi et al. (October 2002); Nature pp. 853-857; Zheng et al. (October 2002); Nature pp. 849-853]. Recently, the PIN1 protein has been found to be upregulated in many types of cancer cells.

Although current therapies have met with some success in the treatment of local and disseminated cancer, there remains a need for improved therapeutic regimens that specifically target cancer with minimal side effects. There is therefore, substantial interest in the development of improved vectors, which target cancer cells ex vivo and in vivo.

### SUMMARY OF THE INVENTION

The present invention provides isolated nucleic acid sequences comprising a transcriptional regulatory (TRE) derived from the sequence upstream of the translational start codon of a PIN1 gene, wherein the TRE is selective for cancer cells.

In one aspect, the PIN1 TRE may be comprised of a nucleotide sequence selected from the group consisting of: (a) the sequence shown in SEQ ID NO:1, SEQ ID NO:45 from about nucleotides (nts) 5 to 297 or SEQ ID NO:46 from about nts 7 to 374; (b) a fragment of the sequence shown in SEQ ID NO:1, SEQ ID NO:45, or SEQ ID NO:46 wherein the fragment has tumor selective transcriptional regulatory activity; (c) a nucleotide sequence having at least 80, 85, 90, 91, 92, 93, 94, 95, 96, 97, 98, 99% or more % identity over its entire length to the sequence shown in SEQ ID NO:1 from about nts 1 to 2221, about nts 1818 to 2221, about nts 1924 to 2221, SEQ ID NO:45 from about nts 5 to 297 or SEQ ID NO:46 from about nts 7 to 374 when compared and aligned for maximum correspondence, as measured using a standard sequence comparison algorithm (described herein) or by visual inspection, wherein the nucleotide sequence has tumor selective transcriptional regulatory activity; and (d) a nucleotide sequence having a full-length complement that hybridizes under stringent conditions to the sequence shown in SEQ ID NO:1 from about nts 1 to 2221, about nts 1818 to 2221, about nts 1924 to 2221, SEQ ID NO:45 from about nts 5 to 297 or SEQ ID NO:46 from about nts 7 to 374, wherein the nucleotide sequence has cancer (tumor) selective transcriptional regulatory activity.

In another embodiment, the PIN1 TRE consists essentially of one of the sequences selected from the group consisting of SEQ ID NO:1 from about nts 1 to 2221 (about −1 to −2221), 1818 to 2221 (about −1 to 404), 1924 to 2221 (about −1 to −298) and SEQ ID NO:45 from about nts 5 to 297 and SEQ ID NO:46 from about nts 7 to 374.

In a related aspect, the invention provides a vector comprising a PIN1 TRE. The vector may be a viral or non-viral vector, which is replication competent or replication defective. The vector may serve as a gene delivery vehicle or the PIN1 TRE may provide for selective replication of the vector in cancer cells.

In one embodiment, the invention provides a replication competent adenovirus vector comprising a first and optionally a second adenovirus gene essential for replication under transcriptional control of a PIN1 TRE.

In another embodiment, the invention provides a replication competent adenovirus vector comprising a first adenovirus gene essential for replication under transcriptional control of a PIN1 TRE and a second adenovirus gene essential for replication under transcriptional control of a different heterologous TRE.

In a further embodiment, the invention provides a replication competent adenovirus vector comprising a transgene wherein the transgene is operably linked to a PIN1 or other heterologous TRE.

The invention further provides a method for selective cytolysis of cancer cells by administering a vector comprising a PIN1 TRE, wherein upon introduction into the cell, the vector replicates and effects selective cytolysis of the cancer cells.

### DESCRIPTION OF THE DRAWINGS

FIGS. 1A and B show the 5′ sequence of the human PIN1 gene (SEQ ID NO:1; GenBank#AF501321), with the start codon, ATG shown as underlined. The transcription initiation site is labeled as +1 and the location of the E2F-binding sites and SP1 binding sites are also indicated in the figure.

FIGS. 2A-W provide a schematic depiction of exemplary adenoviral vectors, wherein FIG. 2A depicts a wild type adenovirus vector which shows adenoviral E1A and E1B genes under control of native E1A and E1B promoters, respectively. FIGS. 2B-W depict exemplary recombinant adenoviral vectors comprising a prolyl isomerase (PIN1) regulatory sequence of the invention. The vectors shown in FIG. 2B-W have at least one of the following characteristics: a Pin1TRE (PIN1) operatively linked to E1A, E1B or E4; an E1b 19 kD deletion, mutation or inactivation (19 k deleted); a heterologous promoter (SP) operatively linked to E1A, E1B or E4; an internal ribosome entry site (IRES) or self-processing cleavage site (SPCS) operatively linked to E1B. The heterologous promoter may be comprised of one or more TRE(s) that are active in a cancer target cell.

### DETAILED DESCRIPTION OF THE INVENTION

The invention provides PIN1 transcriptional regulatory elements (TREs) which preferentially enhance the net transcription of cis operably-linked transcription units in cancer cells. The TREs of the present invention are preferentially active in cancer cells as compared with other tissues. The invention also provides compositions and methods comprising a PIN1 TRE of the invention for therapy of hyperplasia and neoplasia, and methods for selective cytolysis of cancer (tumor) cells using the same. The compositions and methods of the invention rely on the use of polynucleotides comprising a PIN1 TRE, suitable for use as gene-targeting constructs and/or for the expression of transgenes. In one aspect the invention provides a vector comprising a PIN1 TRE of the invention.

General Techniques

The practice of the present invention will employ, unless otherwise indicated, conventional techniques of molecular biology (including recombinant techniques), microbiology, cell biology, biochemistry and immunology, which are within the skill of the art. Such techniques are explained fully in the literature, such as, “Molecular Cloning: A Laboratory Manual”, second edition (Sambrook et al., 1989); “Oligonucleotide Synthesis” (M. J. Gait, ed., 1984); “Animal Cell Culture” (R. I. Freshney, ed., 1987); “Methods in Enzymology” (Academic Press, Inc.); “Handbook of Experimental Immunology” (D. M. Weir & C. C. Blackwell, eds.); “Gene Transfer Vectors for Mammalian Cells” (J. M. Miller & M. P. Calos, eds., 1987); “Current Protocols in Molecular Biology” (F. M. Ausubel et al., eds., 1987); “PCR: The Polymerase Chain Reaction”, (Mullis et al., eds., 1994); and “Current Protocols in Immunology” (J. E. Coligan et al., eds., 1991).

### DEFINITIONS

Unless otherwise indicated, all terms used herein have the same meaning as they would to one skilled in the art and the practice of the present invention will employ, conventional techniques of microbiology and recombinant DNA technology, which are within the knowledge of those of skill of the art.

As used herein, the terms “neoplastic cells”, “neoplasia”, “tumor”, “tumor cells”, “carcinoma”, “carcinoma cells”, “cancer” and “cancer cells”, (used interchangeably) refer to cells which exhibit relatively autonomous growth, so that they exhibit an aberrant growth phenotype characterized by a significant loss of control of cell proliferation. Neoplastic cells can be malignant or benign.

As used herein, “suppressing tumor growth” refers to reducing the rate of growth of a tumor, halting tumor growth completely, causing a regression in the size of an existing tumor, eradicating an existing tumor and/or preventing the occurrence of additional tumors upon treatment with the compositions, kits or methods of the present invention. “Suppressing” tumor growth indicates a growth state that is curtailed when compared to growth without intervention. Tumor cell growth can be assessed by any means known in the art, including, but not limited to, measuring tumor size, determining whether tumor cells are proliferating using a ³H-thymidine incorporation assay, or counting tumor cells. “Suppressing” tumor cell growth means any or all of the following states: slowing, delaying, and stopping tumor growth, as well as tumor shrinkage.

“Delaying development” of a tumor means to defer, hinder, slow, retard, stabilize, and/or postpone development of the disease. This delay can be of varying lengths of time, depending on the history of the disease and/or individual being treated.

The terms “polynucleotide” and “nucleic acid”, used interchangeably herein, refer to a polymeric form of nucleotides of any length, either ribonucleotides or deoxyribonucleotides. These terms include a single-, double- or triple-stranded DNA, genomic DNA, cDNA, RNA, DNA-RNA hybrid, or a polymer comprising purine and pyrimidine bases, or other natural, chemically, biochemically modified, non-natural or derivatized nucleotide bases. Preferably, a vector of the invention comprises DNA. As used herein, “DNA” includes not only bases A, T, C, and G, but also includes any of their analogs or modified forms of these bases, such as methylated nucleotides, internucleotide modifications such as uncharged linkages and thioates, use of sugar analogs, and modified and/or alternative backbone structures, such as polyamides.

The following are non-limiting examples of polynucleotides: a gene or gene fragment, exons, introns, mRNA, tRNA, rRNA, ribozymes, cDNA, recombinant polynucleotides, branched polynucleotides, plasmids, vectors, isolated DNA of any sequence, isolated RNA of any sequence, nucleic acid probes, and primers. A polynucleotide may comprise modified nucleotides, such as methylated nucleotides and nucleotide analogs, uracyl, other sugars and linking groups such as fluororibose and thioate, and nucleotide branches. The sequence of nucleotides may be interrupted by non-nucleotide components. A polynucleotide may be further modified after polymerization, such as by conjugation with a labeling component. Other types of modifications included in this definition are caps, substitution of one or more of the naturally occurring nucleotides with an analog, and introduction of means for attaching the polynucleotide to proteins, metal ions, labeling components, other polynucleotides, or a solid support. Preferably, the polynucleotide is DNA. As used herein, “DNA” includes not only bases A, T, C, and G, but also includes any of their analogs or modified forms of these bases, such as methylated nucleotides, internucleotide modifications such as uncharged linkages and thioates, use of sugar analogs, and modified and/or alternative backbone structures, such as polyamides.

A polynucleotide or polynucleotide region has a certain percentage, for example at least 80, 85, 90, 91, 92, 93, 94, 95, 96, 97, 98, 99% or more sequence identity over its entire length when aligned, comparing the two sequences. The alignment may be carried out and the percent homology or sequence identity determined using software programs known in the art, for example those described in Current Protocols in Molecular Biology (F. M. Ausubel et al., eds., 1987) Supplement 30, section 7.7.18. A preferred alignment program is ALIGN Plus (Scientific and Educational Software, Pennsylvania), preferably using default parameters, which are as follows: mismatch=2; open gap=0; extend gap=2.

As used herein, a “transcriptional response element” or “transcriptional regulatory element”, or “TRE” is a polynucleotide sequence, preferably a DNA sequence, comprising one or more enhancer(s) and/or promoter(s) and/or promoter elements such as a transcriptional regulatory protein response sequence or sequences, which increases transcription of an operably linked polynucleotide in a host cell that allows a TRE to function.

As used herein, a PIN1 TRE is a cancer-specific transcriptional response element, which preferentially directs gene expression in cancer cells. A PIN1 TRE of the invention comprises a promoter and/or enhancer component of the 5′ sequence to a PIN1 gene. A PIN1 TRE comprises an enhancer element and/or promoter element, which may or may not be derived from the same PIN1 gene.

“Under transcriptional control” is a term well understood in the art and indicates that transcription of a polynucleotide sequence, usually a DNA sequence, depends on its being operably (operatively) linked to an element which contributes to the initiation of, or promotes, transcription.

The term “operably linked” and “operatively linked” used interchangeably, relate to the orientation of polynucleotide elements in a functional relationship. A TRE is operably linked to a coding sequence if the TRE regulates (e.g. promotes) transcription of the coding sequence. Operably linked means that the DNA sequences being linked are generally contiguous and, where necessary to join two protein coding regions, contiguous and in the same reading frame. However, since enhancers generally function when separated from the promoter by several kilobases and intronic sequences may be of variable length, some polynucleotide elements may be operably linked but not contiguous.

The term “enhancer” within the meaning of the invention may be any genetic element, e.g., a nucleotide sequence, that increases transcription of a coding sequence operatively linked to a promoter to an extent greater than the transcription activation effected by the promoter itself when operatively linked to the coding sequence, i.e. it increases transcription from the promoter in certain cells or even all cells.

The term “vector”, as used herein, refers to a nucleic acid construct designed for transfer between different host cells. Vectors may be, for example, “cloning vectors” which are designed for isolation, propagation and replication of inserted nucleotides, “expression vectors” which are designed for expression of a nucleotide sequence in a host cell, a “viral vector” which is designed to result in the production of a recombinant virus or virus-like particle, or “shuttle vectors”, which comprise the attributes of more than one type of vector. Any vector for use in gene introduction can be used as a “vector” into which a sequence having TRE activity is introduced. The term vector as it applies to the present invention is used to describe a recombinant vector, e.g., a plasmid, liposome or viral vector (including a replication defective or replication competent viral vector) comprising a PIN1 TRE. Viral vectors, such as retrovirus vectors (e.g. derived from Moloney murine leukemia MoMLV, virus vectors (MoMLV), MSCV, SFFV, MPSV, SNV etc), lentiviral vectors (e.g. derived from HIV-1, HIV-2, SIV, BIV, FIV etc.), adenovirus vectors (including replication competent, replication deficient and gutless forms thereof), or adeno associated virus (AAV) vectors, (simian virus 40 (SV-40) vectors), bovine papilloma virus vectors, Epstein-Barr virus, herpes virus vectors, vaccinia virus vectors, Moloney murine leukemia virus vectors, Harvey murine sarcoma virus vectors, murine mammary tumor virus vectors, and Rous sarcoma virus vectors may be employed in the practice of the present invention.

The terms “virus”, “viral particle”, “vector particle”, “viral vector particle”, and “virion” are used interchangeably and are to be understood broadly as meaning infectious viral particles that are formed when, e.g., a viral vector of the invention is transduced into an appropriate cell or cell line for the generation of infectious particles. Viral particles according to the invention may be utilized for the purpose of transferring nucleic acids (e.g., DNA or RNA) into cells either in vitro or in vivo.

The term “replication defective” as used herein relative to a viral vector of the invention means the viral vector cannot further replicate and package its genomes or does so at negligible levels i.e. several orders of magnitude lower amounts of replication and/or packaging as compared to an unmodified parental virus. For example, when the cell of a subject are infected with rAAV virions, the heterologous gene is expressed in the patient's cells, however, due to the fact that the patient's cells lack AAV rep and cap genes and the adenovirus accessory function genes, the rAAV is replication defective and wild-type AAV cannot be formed in the patient's cells.

As used herein, “packaging system” refers to a set of viral constructs comprising genes that encode viral proteins involved in packaging a recombinant virus. Typically, the constructs of the packaging system will ultimately be incorporated into a packaging cell.

The term “replication competent” as used herein may also be referred to as “replication conditional” relative to a viral vector of the invention. The term means the vector can selectively replicate in particular cell types (“target cells”), e.g., cancer cells and preferentially effect cytolysis of those cells. The term “replication-competent” as used herein relative to the viral vectors of the invention means the viral vectors and particles preferentially replicate in certain types of cells or tissues but to a lesser degree or not at all in other types. In one embodiment of the invention, the viral vector and/or particle selectively replicates in tumor cells and or abnormally proliferating tissue, such as solid tumors and other neoplasms. Such viruses may be referred to as “oncolytic viruses” or “oncolytic vectors” and may be considered to be “cytolytic” or “cytopathic” and to effect “selective cytolysis” of target cells. In one aspect, the present invention provides viruses and viral vectors, which are replication competent and selectively replicate in cells expressing Pin1. The viruses and viral vectors may be derived from any viral source, for example, a virus that can infect and replicate in mammalian cells. Viruses of the invention may be based on (derived from) the following, but are not limited to, herpes virus (WO 01/53506, WO 2000/22137, WO 00/46355, WO 01/41801, WO 00/65078, U.S. Pat. No. 5,585,096, adenovirus (US Patent Publication No.2003-0104625, U.S. Pat. No. 6,692,736; U.S. Pat. No. 6,676,935), e.g., a herpes virus that does not express ICP34.5, reovirus (e.g. rotavirus; WO 99/08692), parvoviruses (WO 97/04805; WO 99/18799, WO 01/12666), papovaviruses (WO 99/18799), iridoviruses (WO 99/18799), hepadenavirus, poxvirus, retroviruses, paramyxovirus (e.g. Newcastle disease virus; WO0120989), mumps virus, human parainfluenza virus; WO9918799), adeno-associated viruses, vaccinia viruses (WO9918799), rhabdovirus (WO9918799), togavirus (e.g. sindbis virus; WO9918799), flavivirus (WO9918799), reovirus (WO9918799), picornavirus (WO9918799), vesicular stomatitis virus (WO9918799; WO0119380), poliovirus (U.S. Pat. No. 6,264,940) and coronavirus (WO9918799).

The term “plasmid” as used herein refers to a DNA molecule that is capable of autonomous replication within a host cell, either extrachromosomally or as part of the host cell chromosome(s). The starting plasmids herein are commercially available, are publicly available on an unrestricted basis, or can be constructed from such available plasmids as disclosed herein and/or in accordance with published procedures. In certain instances, as will be apparent to the ordinarily skilled artisan, other plasmids known in the art may be used interchangeably with plasmids described herein.

The terms “complement” and “complementary” refer to two nucleotide sequences that comprise antiparallel nucleotide sequences capable of pairing with one another upon formation of hydrogen bonds between the complementary base residues in the antiparallel nucleotide sequences.

The term “expression” refers to the transcription and/or translation of an endogenous gene, transgene or coding region in a cell.

By “transcriptional activation” or an “increase in transcription,” it is intended that transcription is increased above basal levels in a normal, i.e. non-transformed cell by at least about 2 fold, preferably at least about 5 fold, preferably at least about 10 fold, more preferably at least about 20 fold, more preferably at least about 50 fold, more preferably at least about 100 fold, more preferably at least about 200 fold, even more preferably at least about 400 fold to about 500 fold, even more preferably at least about 1000 fold. Basal levels are generally the level of activity (if any) in a non-target cell (i.e., a different cell type), or the level of activity (if any) of a reporter construct lacking a PIN1 TRE as tested in a target cell line. When the TRE controls a gene necessary for viral replication or expression of a gene, the replication of virus or expression of a gene is significantly higher in the target cells, as compared to a control cell, usually at least about 2-fold higher, preferably, at least about 5-fold higher, more preferably, at least about 10-fold higher, still more preferably at least about 50-fold higher, even more preferably at least about 100-fold higher, still more preferably at least about 400- to 500-fold higher, still more preferably at least about 1000-fold higher, most preferably at least about 1×10⁶ higher. Most preferably, the TRE controls expression of a viral gene or transgene solely in the target cells (that is, does not replicate or replicates at very low levels in non-target cells).

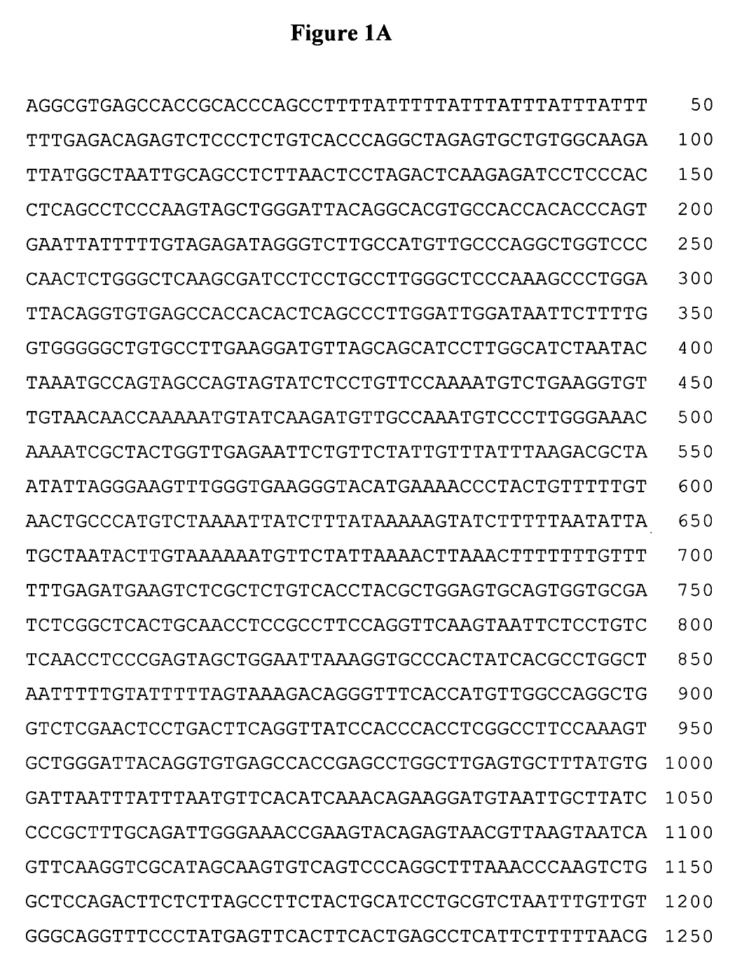

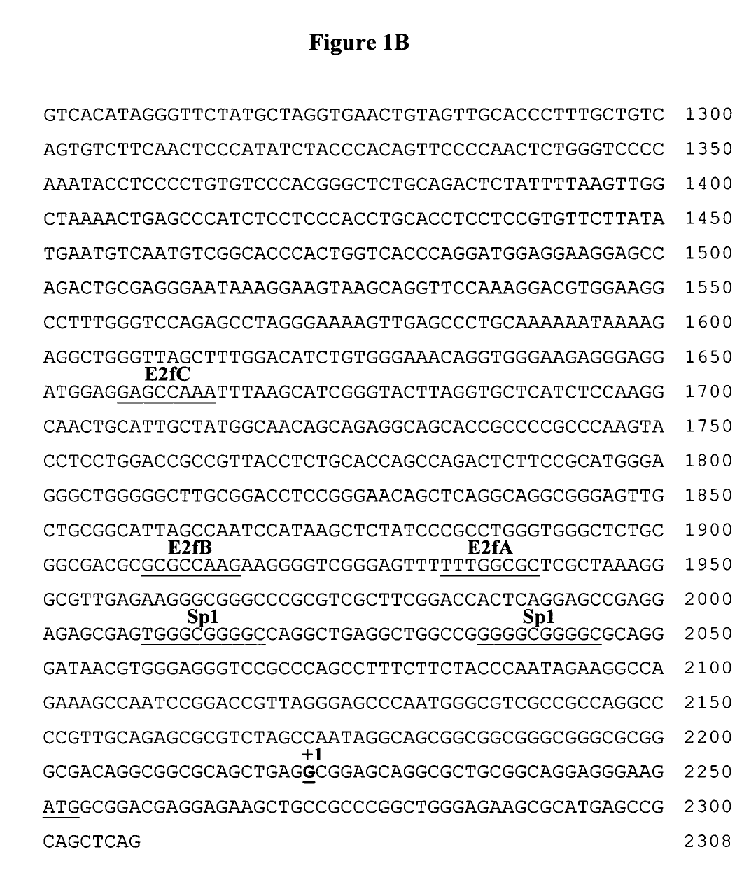

A “termination signal sequence” within the meaning of the invention may be any genetic element that causes RNA polymerase to terminate transcription, such as for example a polyadenylation signal sequence. A polyadenylation signal sequence is a recognition region necessary for endonuclease cleavage of an RNA transcript that is followed by the polyadenylation consensus sequence AATAAA. A polyadenylation signal sequence provides a “polyA site”, i.e. a site on a RNA transcript to which adenine residues will be added by post-transcriptional polyadenylation. Polyadenylation signal sequences are useful insulating sequences for transcription units within eukaryotic cells and eukaryotic viruses. Generally, the polyadenylation signal sequence includes a core poly(A) signal that consists of two recognition elements flanking a cleavage-polyadenylation site (e.g., FIG. 1 of WO 02/067861 and WO 02/068627). The choice of a suitable polyadenylation signal sequence will consider the strength of the polyadenylation signal sequence, as completion of polyadenylation process correlates with poly(A) site strength (Chao et al., Molecular and Cellular Biology, 1999, 19:5588-5600). In principle, any polyadenylation signal sequence may be useful for the purposes of the present invention. In some embodiments of this invention the termination signal sequence is either the SV40 late polyadenylation signal sequence, the SV40 early polyadenylation signal sequence or a bovine growth hormone polyadenylation signal sequence. Usually, the termination signal sequence is isolated from its genetic source and inserted into a vector of the invention at a suitable position upstream of a PIN1 or other heterologous TRE.

A “multicistronic transcript” refers to a mRNA molecule that contains more than one protein coding region, or cistron. A mRNA comprising two coding regions is denoted a “bicistronic transcript.” The “5′-proximal” coding region or cistron is the coding region whose translation initiation codon (usually AUG) is closest to the 5′-end of a multicistronic mRNA molecule. A “5′-distal” coding region or cistron is one whose translation initiation codon (usually AUG) is not the closest initiation codon to the 5′ end of the mRNA. The terms “5′-distal” and “downstream” are used synonymously to refer to coding regions that are not adjacent to the 5′ end of a mRNA molecule.

As used herein, “co-transcribed” means that two (or more) coding regions of polynucleotides are under transcriptional control of a single transcriptional control or regulatory element.

As used herein, an “internal ribosome entry site” or “IRES” refers to an element that promotes direct internal ribosome entry to the initiation codon, such as ATG, of a cistron (a protein encoding region), thereby leading to the cap-independent translation of the gene. See, e.g., Jackson R J, Howell M T, Kaminski A (1990) Trends Biochem Sci 15(12):477-83) and Jackson R J and Kaminski, A. (1995) RNA 1(10):985-1000). The present invention encompasses the use of any IRES element, which is able to promote direct internal ribosome entry to the initiation codon of a cistron. “Under translational control of an IRES” as used herein means that translation is associated with the IRES and proceeds in a cap-independent manner. Examples of “IRES” known in the art include, but are not limited to IRES obtainable from picornavirus (Jackson et al., 1990, Trends Biochem Sci 15(12):477-483); and IRES obtainable from viral or cellular mRNA sources, such as for example, immunoglobulin heavy-chain binding protein (BiP), the vascular endothelial growth factor (VEGF) (Huez et al. (1998) Mol. Cell. Biol. 18(11):6178-6190), the fibroblast growth factor 2, and insulin-like growth factor, the translational initiation factor eIF4G, yeast transcription factors TFIID and HAP4. IRES have also been reported in different viruses such as cardiovirus, rhinovirus, aphthovirus, HCV, Friend murine leukemia virus (FrMLV) and Moloney murine leukemia virus (MoMLV). As used herein, “IRES” encompasses functional variations of IRES sequences as long as the variation is able to promote direct internal ribosome entry to the initiation codon of a cistron. In preferred embodiments, the IRES is mammalian. In other embodiments, the IRES is viral or protozoan. In one illustrative embodiment disclosed herein, the IRES is obtainable from encephelomycarditis virus (ECMV) (commercially available from Novogen, Duke et al. (1992) J. Virol 66(3):1602-1609). In another illustrative embodiment disclosed herein, the IRES is from VEGF. Examples of IRES sequences are described in U.S. Pat. No. 6,692,736.

A “self-processing cleavage site” or “self-processing cleavage sequence” as referred to herein is a DNA or amino acid sequence, wherein upon translation, rapid intramolecular (cis) cleavage of a polypeptide comprising the self-processing cleavage site occurs to result in expression of discrete mature protein or polypeptide products. Such a “self-processing cleavage site”, may also be referred to as a post-translational or co-translational processing cleavage site, e.g., a 2A site, sequence or domain. A 2A site, sequence or domain demonstrates a translational effect by modifying the activity of the ribosome to promote hydrolysis of an ester linkage, thereby releasing the polypeptide from the translational complex in a manner that allows the synthesis of a discrete downstream translation product to proceed (Donnelly, 2001). Alternatively, a 2A site, sequence or domain demonstrates “auto-proteolysis” or “cleavage” by cleaving its own C-terminus in cis to produce primary cleavage products (Furler; Palmenberg, Ann. Rev. Microbiol. 44:603-623 (1990)).

For sequence comparison, typically one sequence acts as a reference sequence to which test sequences are compared. When using a sequence comparison algorithm, test and reference sequences are input into a computer, coordinates are designated if necessary, and sequence algorithm program parameters are designated. The sequence comparison algorithm then calculates the percent sequence identity for the test sequence(s) relative to the reference sequence, based on the designated program parameters.

Optimal alignment of sequences for comparison can be conducted, e.g., by the local homology algorithm of Smith & Waterman, Adv. Appl. Math. 2: 482 (1981), by the homology alignment algorithm of Needleman & Wunsch, J Mol. Biol. 48: 443 (1970), by the search for similarity method of Pearson & Lipman, Proc. Nat'l. Acad. Sci. USA 85: 2444 (1988), by computerized implementations of these algorithms (GAP, BESTFIT, FASTA, and TFASTA in the Wisconsin Genetics Software Package, Genetics Computer Group, 575 Science Dr., Madison, Wis.), by the BLAST algorithm, Altschul et al., J Mol. Biol. 215: 403-410 (1990), with software that is publicly available through the National Center for Biotechnology Information (http://www.ncbi.nlm.nih.gov/), or by visual inspection (see generally, Ausubel et al., infra). For purposes of the present invention, optimal alignment of sequences for comparison is most preferably conducted by the local homology algorithm of Smith & Waterman, Adv. Appl. Math. 2: 482 (1981).

The terms “identical” or percent “identity” in the context of two or more nucleic acid or protein sequences, refer to two or more sequences or subsequences that are the same or have a specified percentage of amino acid residues or nucleotides that are the same, when compared and aligned for maximum correspondence, as measured using one of the sequence comparison algorithms described herein, e.g. the Smith-Waterman algorithm, or by visual inspection.

In one embodiment, a PIN1 TRE according to the present invention has a full-length complement that hybridizes under stringent conditions to the sequence shown in SEQ ID NO:1, the sequence from about −1 to −298 as depicted in FIG. 1, the sequence in SEQ ID NO:45 from about nts 5 to 297 or the sequence in SEQ ID NO:46 from about nts 7 to 374. The phrase “hybridizing to” refers to the binding, duplexing, or hybridizing of a molecule only to a particular nucleotide sequence under stringent conditions when that sequence is present in a complex mixture (e.g., total cellular) DNA or RNA. “Bind(s) substantially” refers to complementary hybridization between a probe nucleic acid and a target nucleic acid and embraces minor mismatches that can be accommodated by reducing the stringency of the hybridization media to achieve the desired detection of the target nucleic acid sequence.

“Stringent hybridization conditions” and “stringent wash conditions” in the context of nucleic acid hybridization experiments such as Southern and Northern hybridizations are sequence dependent, and are different under different environmental parameters. Longer sequences hybridize at higher temperatures. An extensive guide to the hybridization of nucleic acids is found in Tijssen (1993) Laboratory Techniques in Biochemistry and Molecular Biology-Hybridization with Nucleic Acid Probes part 1 chapter 2 “Overview of principles of hybridization and the strategy of nucleic acid probe assays” Elsevier, N.Y. Generally, highly stringent hybridization and wash conditions are selected to be about 5° C. to 20° C. (preferably 5° C.) lower than the thermal melting point (Tm) for the specific sequence at a defined ionic strength and pH. Typically, under highly stringent conditions a probe will hybridize to its target subsequence, but not to other sequences.

The Tm is the temperature (under defined ionic strength and pH) at which 50% of the target sequence hybridizes to a perfectly matched probe. Very stringent conditions are selected to be equal to the Tm for a particular probe. An example of stringent hybridization conditions for hybridization of complementary nucleic acids that have more than 100 complementary residues on a filter in a Southern or northern blot is 50% formamide with 1 mg of heparin at 42° C., with the hybridization being carried out overnight. An example of highly stringent wash conditions is 0.15M NaCl at 72° C. for about 15 minutes. An example of stringent wash conditions is a 0.2×SSC wash at 65° C. for 15 minutes (see, Sambrook, infra, for a description of SSC buffer). Often, a high stringency wash is preceded by a low stringency wash to remove background probe signal. An example medium stringency wash for a duplex of, e.g., more than 100 nucleotides, is 1×SSC at 45° C. for 15 minutes. An example low stringency wash for a duplex of, e.g., more than 100 nucleotides, is 4-6×SSC at 40° C. for 15 minutes. For short probes (e.g., about 10 to 50 nucleotides), stringent conditions typically involve salt concentrations of less than about 1.0M Na ion, typically about 0.01 to 1.0 M Na ion concentration (or other salts) at pH 7.0 to 8.3, and the temperature is typically at least about 30° C. Stringent conditions can also be achieved with the addition of destabilizing agents such as formamide. In general, a signal to noise ratio of 2× (or higher) than that observed for an unrelated probe in the particular hybridization assay indicates detection of a specific hybridization.

The phrase “hybridizing to” refers to the binding, duplexing, or hybridizing of a molecule to a particular nucleotide sequence under stringent conditions when that sequence is present in a complex mixture (e.g., total cellular) DNA or RNA. “Bind(s) substantially” refers to complementary hybridization between a probe nucleic acid and a target nucleic acid and embraces minor mismatches that can be accommodated by reducing the stringency of the hybridization media to achieve the desired detection of the target nucleic acid sequence.

As used herein, “transgene” refers to a polynucleotide that can be expressed, via recombinant techniques, in a non-native environment or heterologous cell under appropriate conditions. The transgene may be derived from the same type of cell in which it is to be expressed, but introduced from an exogenous source, modified as compared to a corresponding native form and/or expressed from a non-native site, or it may be derived from a heterologous cell. “Transgene” is synonymous with “exogenous gene”, “foreign gene” and “heterologous gene”. A transgene may be a therapeutic gene.

As used herein, a “therapeutic” gene refers to a transgene that, when expressed, confers a beneficial effect on the cell or tissue in which it is present, or on a mammal in which the gene is expressed. Examples of beneficial effects include amelioration of a sign or symptom of a condition or disease, prevention or inhibition of a condition or disease, or conferral of a desired characteristic. Therapeutic genes include genes that correct a genetic deficiency in a cell or mammal.

In the context of a vector for use in practicing the present invention, a “heterologous polynucleotide” or “heterologous gene” or “transgene” is any polynucleotide or gene that is not present in the corresponding wild-type vector or virus. Examples of preferred transgenes for inclusion in the vectors of the invention are provided herein below.

In the context of a vector for use in practicing the present invention, a “heterologous” promoter or enhancer is one which is not associated with or derived from the corresponding wild-type vector or virus.

In the context of a PIN1 TRE, a “heterologous” promoter, enhancer or TRE is one which is derived from a gene other than the PIN1 gene.

In the context of a vector for use in practicing the present invention, an “endogenous” promoter, enhancer or TRE is native to or derived from the corresponding wild-type vector or virus.

“Replication” and “propagation” are used interchangeably and refer to the ability of a viral vector of the invention to reproduce or proliferate. These terms are well understood in the art. For purposes of this invention, replication involves production of virus proteins and is generally directed to reproduction of virus. Replication can be measured using assays standard in the art and described herein, such as a virus yield assay, burst assay or plaque assay. “Replication” and “propagation” include any activity directly or indirectly involved in the process of virus manufacture, including, but not limited to, viral gene expression; production of viral proteins, nucleic acids or other components; packaging of viral components into complete viruses and cell lysis.

“Preferential replication” and “selective replication” and “specific replication” may be used interchangeably and mean that the virus replicates more in a target cancer cell than in a non-cancer cell. Preferably, the virus replicates at a significantly higher rate in target cells than non target cells; preferably, at least about 3-fold higher, more preferably, usually at least about 10-fold higher, it may be at least about 50-fold higher, and in some instances at least about 100-fold, 400-fold, 500-fold, 1000-fold or even 1×10⁶ higher. In one embodiment, the virus replicates only in the target cells (that is, does not replicate at all or replicates at a very low level in non-target cells).

An “individual” is a vertebrate, preferably a mammal, more preferably a human. Mammals include, but are not limited to, farm animals, sport animals, rodents, primates, and pets. A “host cell” includes an individual cell or cell culture which can be or has been a recipient of a vector(s) of this invention. Host cells include progeny of a single host cell, and the progeny may not necessarily be completely identical (in morphology or in total DNA complement) to the original parent cell due to natural, accidental, or deliberate mutation and/or change. A host cell includes cells transfected or infected in vivo or in vitro with a vector of this invention.

As used herein, “cytotoxicity” is a term well understood in the art and refers to a state in which a cell's usual biochemical or biological activities are compromised (i.e., inhibited). These activities include, but are not limited to, metabolism; cellular replication; DNA replication; transcription; translation; uptake of molecules. “Cytotoxicity” includes cell death and/or cytolysis. Assays are known in the art which indicate cytotoxicity, such as dye exclusion, ³H-thymidine uptake, and plaque assays.

The terms “selective cytotoxicity” and “specific cytotoxicity” are used interchangeably and as used herein, refer to the cytotoxicity conferred by a vector of the invention on a cell which allows or induces a PIN1 TRE to function (referred to herein as a “target cell”) when compared to the cytotoxicity conferred by a vector of the present invention on a cell which does not allow a PIN1 TRE to function (a “non-target cell”). Such cytotoxicity may be measured, for example, by plaque assays, by reduction or stabilization in size of a tumor comprising target cells, or the reduction or stabilization of serum levels of a marker characteristic of the tumor cells, or a tissue-specific marker, e.g., a cancer marker. Cytotoxicity is a term well understood in the art and refers to a state in which a cell's usual biochemical or biological activities are compromised (i.e., inhibited), including cell death and/or cytolysis. These activities include, but are not limited to, metabolism; cellular replication; DNA replication; transcription; translation; uptake of molecules. Assays known in the art as indicators of cytotoxicity, include dye exclusion, ³H-thymidine uptake, and plaque assays.

The terms “candidate bioactive agent”, “drug candidate” “compound” or grammatical equivalents as used herein describes any molecule, e.g., protein, oligopeptide, small organic molecule, polysaccharide, polynucleotide, vector, e.g. a viral or non-viral (e.g., a plasmid) vector, etc., to be tested for bioactive agents that are capable of directly or indirectly altering the cancer phenotype or the expression of a cancer-associated sequence, including both nucleic acid sequences and protein sequences. In preferred embodiments, the bioactive agents modulate the expression profiles, or expression profile nucleic acids or proteins provided herein. In a particularly preferred embodiment, the candidate agent suppresses a cancer phenotype, for example to a normal tissue fingerprint. Similarly, the candidate agent preferably suppresses a severe cancer phenotype. Generally pluralities of assay mixtures are run in parallel with different agent concentrations to obtain a differential response to the various concentrations. Typically, one of these concentrations serves as a negative control, i.e., at zero concentration or below the level of detection.

PIN1 Transcriptional Response Elements of the Invention

PIN1 has been shown to be overexpressed in many human cancers, e.g., brain tumors including oligodendroglioma, astrocytoma and glioblastomamultiforme); genecological tumors including cervical carcinoma, ovary endometroid cancer, ovarian Brenner tumors, ovarian mucinous cancer, ovarian serous cancer, uterine carcinosarcoma, breast—lobular cancer, breast—ductal cancer, breast—medullary cancer, breast—mucinous cancer and breast—tubular cancer; endocrine tumors including thyroid adenocarcinoma, thyroid follicular cancer, thyroid medullary cancer, thyroid papillary carcinoma, parathyroid—adenocarcinoma and adrenal gland cancer; digestive tract tumors including colon adenoma mild displasia, colon adenoma moderate displasia, colon adenoma severe displasia, colon adenocarcinoma; esophagus adenocarcinoma; hepatocelluar carcinoma; mouth cancer; gall bladder adenocarcinoma; pancreatic adenocarcinoma; genitourinary tract tumors; hormone-refractive prostate cancer; untreated prostate cancer; testis non-seminomatous cancer; testis seminomatous and urinary bladder transitional carcinoma; respiratory tract tumors including lung—adenocarcinoma, lung—large cell cancer, lung—small cell cancer and lung—squamous cell carcinoma; hematological neoplasias including malt lymphoma; NHL diffuse large B, thymoma and NHL (others); skin tumors including malignant melanoma; basolioma; squamous cell cancer; oral squamous cell carcinoma; merkel zell cancer; and skin benign nevus; as well as soft tissue tumors including lipoma and liposarcoma (as described e.g., in U.S. Patent Publication U.S. 2003-0068626).

The expression of PIN1 closely correlates with the tumor grade and cyclin D1 expression level in tumors (Wulf et al., EMBO J 20:3459-3472 (2001)). In addition, overexpression of PIN1 enhances whereas inhibition of PIN1 suppresses transformed phenotypes of mammary epithelial cells induced by Neu and Ras (Ryo et al. Molecular and Cellular Biology, pp. 5281-5295 (2002)). Overexpression of PIN1 in mammary epithelial cells has been shown to result in an anchorage dependent cell growth phenotype. These results suggest that PIN1 overexpression can induce events associated with the stages of mammary tumorigenesis. Therefore, the study of PIN1 and its expression may prove valuable for treating cancer. Also, inhibition of PIN1 expression will have a negative effect on tumor growth and/or metastasis (Ryo et al. 2002).

A PIN1 TRE is a cancer-specific TRE, which preferentially directs gene expression in cancer cells. Analysis conducted by Ryo et al. (Molecular and Cellular Biology, pp. 5281-5295 (2002)) indicate that the transcriptional control unit is located in the 2.3 kb of sequence located upstream of the coding region. The promoter has neither TATA nor CAAT boxes but has two putative GC boxes and three consensus E2F-binding sites named A, B and C (FIG. 1). Deletion and/or mutation of these three sites suggest a repressor role for the distal site (site A) and that activation is heavily dependent on the proximal site (Site C; Ryo et al. 2002). The middle site (Site B) appears to enhance the transcriptional activation of the unit. A range of data demonstrates that the binding sites effectively compete for binding to E2F, that E2F binding to the PIN1 promoter correlates to PIN1 expression, and that PIN1 expression correlates to cell cycles.

A PIN1 TRE of the invention comprises a promoter and/or enhancer component of the sequence 5′ to a PIN1 gene. This region of DNA contains native transcriptional elements that direct expression of the PIN1 gene. A PIN1 TRE of the present invention finds utility in vector-mediated delivery and in vivo expression of polynucleotides encoding proteins that are effective in the treatment of cancer. A PIN1 TRE provides a means for cancer-cell specific replication of a vector comprising a PIN1 TRE and/or cancer-cell specific expression of a gene (e.g., a transgene) operably linked to a PIN1 TRE.

In addition to the PIN1 TRE, a vector for use in practicing the invention may further comprise promoters and/or enhancers derived from the same or different genes. Such additional regulatory elements may be operably linked to a viral gene essential for replication or to a transgene.

A PIN1 TRE comprises a mammalian cancer-specific enhancer and/or promoter. Preferred PIN1 TREs comprise a PIN1 enhancer and/or promoter and are of human, primate, rat or mouse origin, including promoter and enhancer elements and transcription factor binding sequences from the 5′ PIN1 sequence set forth in SEQ ID NO:1. The term “PIN1 promoter” refers to the native PIN1 promoter and functional fragments, mutations and derivatives thereof. A PIN1 TRE contains the native promoter elements that direct expression of an operably linked gene. Usually a promoter region will have at least about 100 nt of sequence located 5′ to the gene and may further comprise, but not always, a TATA box and/or CAAT box motif sequence. The native human PIN1 promoter does not have a recognizable TATA or CAAT box. In one embodiment, a PIN1 TRE and a heterologous CAAT and/or heterologous TATA box are operatively linked to a coding region.

A PIN1 TRE of the invention may or may not include the full-length wild type promoter and/or enhancer. One skilled in the art knows how to derive fragments from a PIN1 TRE and test them for the desired specificity. A PIN1 promoter fragment of the present invention has promoter activity specific for tumor cells, i.e. drives tumor selective expression of an operatively linked coding sequence. In one embodiment, the PIN1 TRE of the invention is a mammalian PIN1 TRE and in another embodiment it is a human PIN1 (hPIN1) TRE, examples of which are further described herein.

The sequence of the 5′ region of the PIN gene, and further 5′ upstream sequences may be utilized to direct gene expression, in tissues where PIN1 is expressed, e.g. carcinoma cells and silencer regions which inhibit expression in tissues where PIN1 is not expressed or expressed at low levels. Sequence alterations, including substitutions, deletions and additions, may be introduced into a PIN TRE to determine the effect of altering expression in experimentally defined systems. Methods for the identification of specific DNA motifs involved in the binding of transcriptional factors are known in the art, e.g. sequence similarity to known binding motifs, gel retardation studies, etc. Preferential replication in cancer cells is determined by conducting assays that compare replication of the vector in a cancer cell which allows function of the PIN1 TREs with replication in a non-cancer cell which does not or the function is at a much lower level.

PIN1 regulatory sequences may be used to identify cis acting sequences required for transcriptional or translational regulation of PIN1 expression, i.e., in different stages of metastasis, and to identify cis acting sequences and trans acting factors that regulate or mediate expression. Such transcription or translational control regions may be operably linked to a gene of interest in order to promote expression of a protein of interest in cultured cells, or in embryonic, fetal or adult tissues, and for gene therapy.

A PIN1 TRE can also comprise multimers. For example, a PIN1 TRE can comprise a tandem series of at least two, at least three, at least four, or at least five promoter fragments alone or in combination with one or more enhancers. These multimers may also contain a heterologous promoter and/or enhancer sequence and/or transcription factor binding sites which are not derived from 5′ sequences upstream of the translational start of a PIN1 gene. A PIN1 TRE maybe modified to retain certain elements or fragments that retain cancer cell specificity while having regions deleted that do not play a significant role in cancer specific transcription. Thus creating a smaller sequence containing one or more PIN1 TRE(s). This embodiment is useful in that some viral vectors (e.g. adenoviral vectors) have a finite packaging capacity. Therefore, decreasing the size of the PIN1 TRE(s) allows for other sequences to be incorporated into the vector or may allow the modified TRE(s) to be incorporated into the viral vector and packaged when it would not otherwise be possible.

The promoter, enhancer and/or transcription factor binding site components of a PIN1 TRE may be in any orientation and/or distance from the coding sequence of interest, as long as the desired target cell-specific transcriptional activity is obtained. Transcriptional activation can be measured in a number of ways known in the art, but is generally measured by detection and/or quantitation of mRNA or the protein product of the coding sequence under control of (i.e., operably linked to) the PIN1 TRE.

In cases where an entire gene, including its own initiation codon and adjacent sequences, is inserted into the appropriate expression vector, no additional translational control signals may be needed. However, in cases where only a portion of the gene coding sequence is inserted, exogenous translational control signals, including, perhaps, the ATG initiation codon, must be provided. Furthermore, the initiation codon must be in phase with the reading frame of the desired coding sequence to ensure translation of the entire insert. These exogenous translational control signals and initiation codons can be of a variety of origins, both natural and synthetic. The efficiency of expression may be enhanced by the inclusion of appropriate transcription enhancer elements, transcription terminators, etc. (see Bittner et al., 1987, Methods in Enzymol. 153:516-544). In some embodiments, specificity is conferred by preferential replication of the vector in target cells due to the PIN1 TRE driving transcription of a gene essential for replication. In other embodiments, efficacy is conferred by preferential transcription and/or translation of a transgene due to operable linkage to a PIN1 TRE.

In other words, the present invention relies upon the cancer-specific expression of a coding sequence operatively linked to a PIN1 TRE and the use of vectors comprising a PIN1 TRE as a means for targeting/expression of operably linked coding sequences in cancer cells. Such targeting may relate to replication of the vector and/or expression of a transgene encoded therein.

In one embodiment, the PIN1 TRE may be comprised of a nucleotide sequence selected from the group consisting of: (a) the sequence shown in SEQ ID NO:1, SEQ ID NO:45 from about nucleotides (nts) 5 to 297 or SEQ ID NO:46 from about nts 7 to 374; (b) a fragment of the sequence shown in SEQ ID NO:1, SEQ ID NO:45, or SEQ ID NO:46 wherein the fragment has tumor selective transcriptional regulatory activity; (c) a nucleotide sequence having at least 80, 85, 90, 91, 92, 93, 94, 95, 96, 97, 98, 99% or more % identity over its entire length to the sequence shown in SEQ ID NO:1 from about nts 1 to 2221, about nts 1818 to 2221, about nts 1924 to 2221, SEQ ID NO:45 from about nts 5 to 297 or SEQ ID NO:46 from about nts 7 to 374 when compared and aligned for maximum correspondence, as measured using a standard sequence comparison algorithm (described herein) or by visual inspection, wherein the nucleotide sequence has tumor selective transcriptional regulatory activity; and (d) a nucleotide sequence having a full-length complement that hybridizes under stringent conditions to the sequence shown in SEQ ID NO:1 from about nts 1 to 2221, about 1818 to 2221, about nts 1924 to 2221, SEQ ID NO:45 from about nts 5 to 297 or SEQ ID NO:46 from about nts 7 to 374 wherein the nucleotide sequence has tumor selective transcriptional regulatory activity. In another embodiment, the PIN1 TRE consists essentially of one of the sequences selected from the group consisting of SEQ ID NO:1 from about nts 1 to 2221 (about −1 to −2221), 1818 to 2221 (about −1to 404), 1924 to 2221 (about −1 to −298) and SEQ ID NO:45 from about nts 5 to 297 and SEQ ID NO:46 from about nts 7 to 374.

As discussed herein, a PIN1 TRE of the invention can be of varying lengths, and of varying sequence composition. Preferably, a given % sequence identity exists over a region of the sequences that is at least about 50 nucleotides in length, more preferably over a region of at least about 100 nucleotides, and even more preferably over a region of at least about 200 nucleotides. Most preferably, the given % sequence identity exists over the entire length of the sequences. In another embodiment of a recombinant viral vector of the invention, the PIN1 TRE sequence consists essentially of SEQ ID NO:1 from about nts 1924 to 2221 (about −1 to −298).

The invention contemplates functionally preserved variants of a PIN1 TRE sequences disclosed herein. Variant PIN1 TREs retain function in the target cell but need not exhibit maximal function. In fact, maximal transcriptional activation activity of a PIN1 TRE may not always be necessary to achieve a desired result, and the level of induction afforded by a fragment of a PIN1 TRE may be sufficient for certain applications. Also, included in the present invention are variants of a PIN1 TRE which displays higher activity, are more responsive and/or are more selective. Examples of functionally preserved variants include those comprising mutations which modify ATG sequences. (See, e.g. SEQ ID Nos: 45 and 46.)

As discussed herein, a PIN1 TRE can be of varying lengths, and of varying sequence composition. The size of a PIN1 TRE is determined in part by the capacity of the vector, which in turn depends upon the contemplated form of the vector. Generally minimal sizes are preferred for PIN 1 TREs, as this provides potential room for insertion of other sequences which may be desirable, such as transgenes, and/or additional regulatory sequences. In a preferred embodiment, such an additional regulatory sequence is an IRES or a self-processing cleavage sequence, such as a 2A sequence. However, the invention contemplates the use of larger and full length PIN TREs.

To minimize non-specific replication, endogenous viral TREs may be removed from the vector. Besides facilitating target cell-specific replication, removal of endogenous TREs also provides greater insert capacity in the vector. Even more importantly, deletion of endogenous TREs prevents the possibility of a recombination event whereby a heterologous TRE is deleted and the endogenous TRE assumes transcriptional control of its respective virus coding sequences. However, endogenous TREs can be maintained in the vector(s), provided that sufficient cell-specific replication preference is preserved.

In another aspect, methods are provided for conferring selective cytotoxicity in target cancer cells by contacting the cells with a viral vector of the invention, whereby the vector enters the cell and propagates. The replication of viral vectors comprising a PIN1 TRE in cancer cells, as compared to non-cancer cells, or to normal, i.e. non-transformed cells, is at least about 3 fold greater and is usually about 10 fold greater, and may be about 100 fold greater, and in some instances is as much as about 1000 fold or more greater. The administration of virus may be combined with additional treatment(s) appropriate to the particular disease, e.g. antiviral therapy, chemotherapy, surgery, radiation therapy or immunotherapy. In some embodiments, this treatment suppresses tumor growth, e.g. by killing tumor cells. In other embodiments, the size and/or extent of a tumor is reduced, or its development delayed.

The term “composite TRE” refers to a TRE that comprises transcriptional regulatory elements that are not naturally found together, usually providing a non-native combination of promoters and enhancer, for example, a heterologous combination of promoter and enhancer and/or transcription factor binding sites; a combination of human and mouse promoter and enhancer; two or more enhancers in combination with a promoter; multimers of the foregoing; and the like. At least one of the promoter, enhancer or and/or transcription factor binding site elements will be cancer specific, for example a PIN1 TRE in combination with an enhancer. In other embodiments, two or more of the elements will provide cancer specificity. A composite TRE comprising regulatory elements from two or more sources may be used to regulate one or more genes. In one embodiment, the PIN1 TRE is a composite TRE.

A TRE of the present invention may or may not be inducible. As is known in the art, the activity of TREs can be inducible. Inducible TREs generally exhibit low activity in the absence of inducer, and are up-regulated in the presence of inducer. Inducers include, for example, nucleic acids, polypeptides, small molecules, organic compounds and/or environmental conditions such as temperature, pressure or hypoxia. Inducible TREs may be preferred when expression is desired only at certain times or at certain locations, or when it is desirable to titrate the level of expression using an inducing agent.

A TRE for use in the present vectors may or may not comprise a silencer. The presence of a silencer (i.e., a negative regulatory element known in the art) can assist in shutting off transcription (and thus replication) in non-target cells. Thus, the presence of a silencer can confer enhanced cell-specific vector replication by more effectively preventing replication in non-target cells. Alternatively, the lack of a silencer may stimulate replication in target cells, thus conferring enhanced target cell-specificity. The silencer may be derived from a 5′ sequence of a PIN1 gene, may be derived from a gene other than a 5′ sequence of a PIN1 gene or a TRE of the invention may comprise both a silencer from PIN1 and a heterologous silencer.

A “functionally-preserved variant” of a PIN1 TRE differs, usually in sequence, but still retains the biological activity, e.g., cancer cell-specific transcriptional activity of the corresponding native or parent PIN1 TRE, although the degree of activation may be altered. The difference in sequence may arise from, for example, single base mutation(s), addition(s), deletion(s), and/or modification(s) of the bases. The difference can also arise from changes in the sugar(s), and/or linkage(s) between the bases of a PIN1 TRE. For example, certain point mutations within sequences of TREs have been shown to decrease transcription factor binding and stimulation of transcription (see Blackwood, et al. (1998) Science 281:60-63, and Smith et al. (1997) J. Biol. Chem. 272:27493-27496). Certain mutations are also capable of increasing TRE activity. Testing the effect of altered bases may be performed in vitro or in vivo by any method known in the art, such as mobility shift assays, or transfecting vectors containing these alterations in TRE functional and TRE non-functional cells. Additionally, one of skill in the art would recognize that point mutations and deletions can be made to a TRE sequence without altering the ability of the sequence to regulate transcription. It will be appreciated that typically, but not necessarily, a “functionally-preserved variant” of a PIN1 TRE will hybridize to the parent sequence under conditions of high stringency. Exemplary high stringency conditions include hybridization at about 65° C. in about 5×SSPE and washing at about 65° C. in about 0.1×SSPE (where 1×SSPE=0.15 sodium chloride, 0.010 M sodium phosphate, and 0.001 M disodium EDTA). Further examples of high stringency conditions are provided in: Maniatis, et al., Molecular Cloning: A Laboratory Manual, 2nd Edition (1989); and Ausubel, F. M., et al., Eds., Current Protocols in Molecular Biology, John Wiley & Sons, Inc., Copyright (c)1987, 1988, 1989, 1990 by Current Protocols.

In some instances, a “functionally-preserved variant” of a PIN1 TRE is a fragment of a native or parent PIN1 TRE. The term “fragment,” when referring to a PIN1 TRE, refers to a sequence that is the same as part of, but not all of, the nucleic acid sequence of a native or parental PIN1 TRE. Such a fragment either exhibits essentially the same biological function or activity as the native or parental PIN1 TRE; for example, a fragment which retains the cancer cell-specific transcription activity of the corresponding native or parent PIN1 TRE, although the degree of activation may be altered.

Activity of a TRE can be determined, for example, as follows. A TRE polynucleotide sequence or set of such sequences can be generated using methods known in the art, such as chemical synthesis, site-directed mutagenesis, PCR, and/or recombinant methods. The sequence(s) to be tested can be inserted into a vector containing a promoter (if no promoter element is present in the TRE) and an appropriate reporter gene encoding a reporter protein, including, but not limited to, chloramphenicol acetyl transferase (CAT), β-galactosidase (encoded by the lacZ gene), luciferase (encoded by the luc gene), alkaline phosphatase (AP), green fluorescent protein (GFP), and horseradish peroxidase (HRP). Such vectors and assays are readily available, from, inter alia, commercial sources. Plasmids thus constructed are transfected into a suitable host cell to test for expression of the reporter gene as controlled by the putative TRE using transfection methods known in the art, such as calcium phosphate precipitation, electroporation, liposomes, DEAE dextran-mediated transfer, particle bombardment or direct injection. TRE activity is measured by detection and/or quantitation of reporter gene-derived mRNA and/or protein. The reporter gene protein product can be detected directly (e.g., immunochemically) or through its enzymatic activity, if any, using an appropriate substrate. Generally, to determine cell specific activity of a TRE, a TRE-reporter gene construct is introduced into a variety of cell types. The amount of TRE activity is determined in each cell type and compared to that of a reporter gene construct lacking the TRE. A TRE is determined to be cell-specific if it is preferentially functional in one cell type, compared to a different cell type.

Gene Transfer Vectors of the Invention

The present invention contemplates the use of any vector for introduction into mammalian cells. The vector relies on a PIN1 TRE of the invention to effect cancer specific expression of an operably linked gene. Exemplary vectors include but are not limited to, viral and non-viral vectors, such as retroviral vectors, e.g. derived from Moloney murine leukemia virus (MoMLV), and related vectors, e.g., MSCV, SFFV, MPSV, SNV, etc.; lentiviral vectors (e.g. derived from HIV-1, HIV-2, SIV, BIV, FIV, etc.; adenoviral (Ad) vectors including replication competent, replication deficient and gutless forms thereof; adeno-associated viral (AAV) vectors; simian virus 40 (SV-40) vectors; bovine papilloma virus vectors; Epstein-Barr virus vectors; herpes virus vectors; vaccinia virus vectors; Harvey murine sarcoma virus vectors; murine mammary tumor virus vectors; Rous sarcoma virus vectors; and nonviral plasmids. In one preferred approach, the vector is a viral vector. Viral vectors can efficiently transduce cells and introduce their own DNA into a host cell. In generating recombinant viral vectors, non-essential genes are typically replaced with a gene or coding sequence for a heterologous (or non-native) protein.

Methods that are well known to those skilled in the art and can be used to construct expression vectors containing coding sequences and appropriate transcriptional and translational control signals, including a cancer specific control signal, for specific expression of an exogenous gene when introduced into a cell. These methods include, for example, in vitro recombinant DNA techniques, synthetic techniques, and in vivo genetic recombination. Alternatively, RNA capable of encoding gene product sequences may be chemically synthesized using, for example, synthesizers. See, for example, the techniques described in “Oligonucleotide Synthesis”, 1984, Gait, M. J. ed., IRL Press, Oxford. In constructing viral vectors, non-essential genes may be replaced with one or more genes encoding one or more therapeutic compounds or factors. Typically, the vector comprises an origin of replication and the vector may or may not also comprise a “marker” or “selectable marker” function by which the vector can be identified and selected. While any selectable marker can be used, selectable markers for use in expression vectors are generally known in the art and the choice of the proper selectable marker will depend on the host cell. Examples of selectable marker genes which encode proteins that confer resistance to antibiotics or other toxins include ampicillin, methotrexate, tetracycline, neomycin (Southern et al., J., J Mol Appl Genet. 1982;1(4):327-41 (1982)), mycophenolic acid (Mulligan et al., Science 209:1422-7 (1980)), puromycin, zeomycin, hygromycin (Sugden et al., Mol Cell Biol. 5(2):410-3 (1985)) or G418.

Reference to a vector or other DNA sequences as “recombinant” merely acknowledges the operable linkage of DNA sequences that are not typically operably linked as isolated from or found in nature. Regulatory (expression/control) sequences are operatively linked to a nucleic acid coding sequence when the expression/control sequences regulate the transcription and, as appropriate, translation of the nucleic acid sequence. Thus expression/control sequences can include transcriptional regulatory elements, e.g., promoters and enhancers; transcription terminator; a start codon (i.e., ATG) in front of the coding sequence; splicing signal for introns and stop codons, etc.

Adenoviral Vectors

In one aspect, the invention provides an adenoviral vector comprising a PIN1 TRE. The adenoviral vector may be replication defective or replication competent. In the case of replication competent adenoviral vectors, the vector comprises an adenovirus gene essential for replication, e.g. an early gene, under the transcriptional control of a PIN1 TRE. By providing a vector comprising a PIN1 TRE controlling an adenoviral gene essential for replication, specific replication in and corresponding cytotoxicity to cancer cells results.

As used herein, the terms “adenovirus” and “adenoviral particle” are used to include any and all viruses that may be categorized as an adenovirus, including any adenovirus that infects a human or an animal, including all groups, subgroups, and serotypes. Thus, as used herein, “adenovirus” and “adenovirus particle” refer to the virus itself or derivatives thereof and cover all serotypes and subtypes and both naturally occurring and recombinant forms, except where indicated otherwise. Such adenoviruses may be wild type or may be modified in various ways known in the art or as disclosed herein. Such modifications include modifications to the adenovirus genome that is packaged in the particle in order to make an infectious virus. Such modifications include deletions known in the art, such as deletions in one or more of the E1A, E1B, E2A, E2B, E3, or E4 coding regions. The terms also include replication-specific adenoviruses; that is, viruses that preferentially replicate in certain types of cells or tissues but to a lesser degree or not at all in other types. Such viruses are sometimes referred to as “cytolytic” or “cytopathic” viruses (or vectors), and, if they have such an effect on neoplastic cells, are referred to as “oncolytic” viruses (or vectors).

A “replication competent adenovirus vector” or “replication competent adenoviral vector” (used interchangeably) of the invention is a polynucleotide construct, which exhibits preferential replication in primary cancer cells and contains a PIN1 TRE linked to an adenoviral gene. In some embodiments, an adenoviral vector of the invention includes a transgene, e.g., a therapeutic gene such as a cytokine gene. Exemplary adenoviral vectors of the invention include, but are not limited to, DNA, DNA encapsulated in an adenovirus coat, adenoviral DNA packaged in another viral or viral-like form (such as herpes simplex, and AAV), adenoviral DNA encapsulated in liposomes, adenoviral DNA complexed with polylysine, adenoviral DNA complexed with synthetic polycationic molecules, conjugated with transferrin, or complexed with a compound such as PEG to immunologically “mask” the antigenicity and/or increase half-life, or conjugated to a nonviral protein.

An adenoviral vector comprising a PIN1 TRE may further comprise one or more regulatory sequences, e.g. enhancers, promoters, transcription factor binding sites, which may be derived from the same or different genes. The adenovirus vector may comprise co-transcribed first and second genes under control of a PIN1 TRE, wherein the second gene may be under translational control of an internal ribosome entry site (IRES) or a self-processing cleavage sequence, such as a 2A sequence. In some cases, the adenovirus vectors comprise more than two co-transcribed genes under control of a PIN1 TRE. The adenovirus vectors of the invention may or may not comprise the adenoviral E3 region, an E3 sequence, or a portion thereof.

In cases where an adenovirus is used as an expression vector, the coding sequence of interest may be ligated to an adenovirus transcription/translation control complex, e.g., the late promoter and tripartite leader sequence. This chimeric gene may then be inserted in the adenovirus genome by in vitro or in vivo recombination or via standard molecular biological techniques. Insertion in a non-essential region of the viral genome (e.g., region E3) will result in a recombinant virus that is viable and capable of expressing the gene product in infected hosts (see Logan & Shenk, 1984, Proc. Natl. Acad. Sci. USA 81:3655-3659). Standard systems for generating adenoviral vectors for expression of inserted sequences are available from commercial sources, for example the Adeno-X™ expression system from Clontech (Clontechniques (January 2000) p. 10-12).

In one preferred aspect, the adenoviral vectors described herein are replication-competent adenoviral vectors that preferentially replicate in cancer cells comprising an adenovirus gene, preferably a gene essential for replication under transcriptional control of a PIN1 TRE. In general, the adenoviral gene essential for replication is an early gene, e.g. one or more of E1A, E1B and E4.

The adenoviral E1B 19-kDa region refers to the genomic region of the adenovirus E1B gene encoding the E1B 19-kDa product. According to wild-type Ad5, the E1B 19-kDa region is a 261 bp region located between nucleotide 1714 and nucleotide 2244. The E1B 19-kDa region has been described in, for example, Rao et al., Proc. Natl. Acad. Sci. USA, 89:7742-7746. The present invention encompasses deletion of part or all of the E1B 19-kDa region as well as embodiments wherein the E1B 19-kDa region is mutated, as long as the deletion or mutation lessens or eliminates the inhibition of apoptosis associated with E1B-19kDa.

The invention further provides a recombinant adenovirus particle comprising a recombinant adenoviral vector according to the invention. In one embodiment, a capsid protein of the adenovirus particle comprises a targeting ligand. In another embodiment, the capsid protein is a fiber protein. In one aspect, the capsid protein is a fiber protein and the ligand is in the HI loop of the fiber protein. The adenoviral vector particle may also include other mutations to the fiber protein. Examples of these mutations include, but are not limited to those described in U.S. Application No. 20040002060, WO 98/07877, WO 01/92299, and U.S. Pat. Nos. 5,962,311, 6,153,435, and 6,455,314. These include, but are not limited to, mutations that decrease binding of the viral vector particle to a particular cell type or more than one cell type, enhance the binding of the viral vector particle to a particular cell type or more than one cell type and/or reduce the immune response to the adenoviral vector particle in an animal. In addition, the adenoviral vector particles of the present invention may also contain mutations to other viral capsid proteins. Examples of these mutations include, but are not limited to those described in U.S. Pat. Nos. 5,731,190, 6,127,525, and 5,922,315. Other mutated adenoviruses are described in U.S. Pat. Nos. 6,057,155, 5,543,328 and 5,756,086.

The adenovirus vectors of this invention can be prepared using recombinant techniques that are standard in the art. Generally, a PIN1 TRE is inserted 5′ to the adenoviral gene of interest, e.g. an adenoviral replication gene, including one or more early replication genes (although late gene(s) can be used). A PIN1 TRE can be prepared using oligonucleotide synthesis (if the sequence is known) or recombinant methods (such as PCR and/or restriction enzymes). Convenient restriction sites, either in the natural adeno-DNA sequence or introduced by methods such as PCR or site-directed mutagenesis, provide an insertion site for a PIN1 TRE. Accordingly, convenient restriction sites for annealing (i.e., inserting) a PIN1 TRE can be engineered onto the 5′ and 3′ ends of a PIN1 TRE using standard recombinant methods, such as PCR. In one embodiment, the TRE replaces at least one native adenovirus TRE.

Adenoviral vectors containing at least one gene essential for replication (e.g., E1A) under transcriptional control of a PIN1 TRE, are conveniently prepared by homologous recombination or in vitro ligation of two plasmids, one providing the left-hand portion of adenovirus and the other plasmid providing the right-hand region, one or more of which contains at least one adenovirus gene under control of a PIN1 TRE. If homologous recombination is used, the two plasmids should share at least about 500 bp of sequence overlap, although smaller regions of overlap will recombine, but usually with lower efficiencies. Each plasmid, as desired, may be independently manipulated, followed by cotransfection in a competent host, providing complementing genes as appropriate, or the appropriate transcription factors for initiation of transcription from a PIN1 TRE for propagation of the adenovirus. Plasmids are generally introduced into a suitable host cell (e.g. 293, PerC.6, Hela-S3 cells) using appropriate means of transduction, such as cationic liposomes or calcium phosphate. Alternatively, in vitro ligation of the right and left-hand portions of the adenovirus genome can also be used to construct recombinant adenovirus derivative containing all the replication-essential portions of adenovirus genome. Berkner et al. (1983) Nucleic Acid Research 11: 6003-6020; Bridge et al. (1989) J. Virol. 63: 631-638.

For convenience, plasmids are available that provide the necessary portions of adenovirus. Plasmid pXC.1 (McKinnon (1982) Gene 19:33-42) contains the wild-type left-hand end of Ad5. pBHG10 (Bett et al. (1994); Microbix Biosystems Inc., Toronto) provides the right-hand end of Ad5, with a deletion in E3. The deletion in E3 provides room in the virus to insert up to about a 3 KB TRE without deleting the endogenous enhancer/promoter. The gene for E3 is located on the opposite strand from E4 (r-strand). pBHG11 provides an even larger E3 deletion (an additional 0.3 kb is deleted). Bett et al. (1994). Alternatively, the use of pBHGE3 (Microbix Biosystems, Inc.) provides the right hand end of Ad5, with a full-length of E3.

For manipulation of the early genes, the transcription start site of Ad5 E1A is at 498 and the ATG start site of the E1A coding segment is at 560 in the virus genome. This region can be used for insertion of a PIN1 TRE. A restriction site may be introduced by employing polymerase chain reaction (PCR), where the primer that is employed may be limited to the Ad5 genome, or may involve a portion of the plasmid carrying the Ad5 genomic DNA. For example, where pBR322 is used, the primers may use the EcoRI site in the pBR322 backbone and the XbaI site at nt 1339 of Ad5. By carrying out the PCR in two steps, where overlapping primers at the center of the region introduce a nucleotide sequence change resulting in a unique restriction site, one can provide for insertion of a PIN1 TRE at that site.

A similar strategy may also be used for insertion of a PIN1 TRE element in operative linkage to E1B. The E1B promoter of Ad5 consists of a single high-affinity recognition site for Spl and a TATA box. This region extends from Ad5 nt 1636 to 1701. By insertion of a TRE in this region, one can provide for cell-specific transcription of the E1B gene. By employing the left-hand region modified with the cell-specific response element regulating E1A, as the template for introducing a PIN1 TRE to regulate E1B, the resulting adenovirus vector will be dependent upon the cell-specific transcription factors for expression of both E1A and E1B. In some embodiments, part or all of the 19-kDa region of E1B is deleted.

Similarly, a PIN1 TRE can be inserted upstream of the E2 gene to make its expression cell-specific. The E2 early promoter, mapping in Ad5 from 27050-27150, consists of a major and a minor transcription initiation site, the latter accounting for about 5% of the E2 transcripts, two non-canonical TATA boxes, two E2F transcription factor binding sites and an ATF transcription factor binding site (for a detailed review of the E2 promoter architecture see Swaminathan et al., Curr. Topics in Micro. and Immunol. (1995) 199(part 3):177-194.

The E2 late promoter overlaps with the coding sequences of a gene encoded by the counterstrand and is therefore not amenable for genetic manipulation. However, the E2 early promoter overlaps only for a few base pairs with sequences coding for a 33 kD protein on the counterstrand. Notably, the SpeI restriction site (Ad5 position 27082) is part of the stop codon for the above mentioned 33 kD protein and conveniently separates the major E2 early transcription initiation site and TATA-binding protein site from the upstream transcription factor binding sites E2F and ATF. Therefore, insertion of a PIN1 TRE having SpeI ends into the SpeI site in the 1-strand would disrupt the endogenous E2 early promoter of Ad5 and should allow cell-restricted expression of E2 transcripts.

For E4, one must use the right hand portion of the adenovirus genome. The E4 transcription start site is predominantly at about nt 35605, the TATA box at about nt 35631 and the first AUG/CUG of ORF I is at about nt 35532. Virtanen et al. (1984) J. Virol. 51: 822-831. Using any of the above strategies for the other genes, a PIN1 TRE may be introduced upstream from the transcription start site. For the construction of full-length adenovirus with a PIN1 TRE inserted in the E4 region, the co-transfection and homologous recombination are performed in W162 cells (Weinberg et al. (1983) Proc. Natl. Acad. Sci. 80:5383-5386) which provide E4 proteins in trans to complement defects in synthesis of these proteins.

An “E3 region” (used interchangeably with “E3”) is a term well understood in the art and means the region of the adenoviral genome that encodes the E3 gene products. Generally, the E3 region is located between about nucleotides 28583 and 30470 of the adenoviral genome. The E3 region has been described in various publications, including, for example, Wold et al. (1995) Curr. Topics Microbiol. Immunol. 199:237-274. A “portion” of the E3 region means less than the entire E3 region, and as such includes polynucleotide deletions as well as polynucleotides encoding one or more polypeptide products of the E3 region. A recombinant adenoviral vector of the invention may comprise a mutation or deletion in an E3 coding region, such as E3-6.7, KDa, gp19KDa, 11.6KDa (ADP), 10.4 KDa (RIDα), 14.5 KDa (RIDβ), and E3-14.7Kda. See, e.g., WO200102540, WO9955831 and US2003/0104625, each of which is expressly incorporated by reference herein.

Adenoviral constructs containing an E3 region can be generated wherein homologous recombination between an E3-containing adenoviral plasmid, for example, BHGE3 (Microbix Biosystems Inc., Toronto) and a non-E3-containing adenoviral plasmid, is carried out.

Alternatively, an adenoviral vector comprising an E3 region can be introduced into cells, for example 293 cells, along with an adenoviral construct or an adenoviral plasmid construct, where they can undergo homologous recombination to yield adenovirus containing an E3 region. In this case, the E3-containing adenoviral vector and the adenoviral construct or plasmid construct contain complementary regions of adenovirus, for example, one contains the left-hand and the other contains the right-hand region, with sufficient sequence overlap as to allow homologous recombination.

Alternatively, an E3-containing adenoviral vector of the invention can be constructed using other conventional methods including standard recombinant methods (e.g., using restriction nucleases and/or PCR), chemical synthesis, or a combination of any of these. Further, deletions of portions of the E3 region can be created using standard techniques of molecular biology.

In some embodiments, the adenovirus death protein (ADP), encoded within the E3 region, is maintained in an adenovirus vector. The ADP gene, under control of the major late promoter (MLP), appears to code for a protein (ADP) that is important in expediting host cell lysis. Tollefson et al. (1996) J. Virol. 70(4):2296; Tollefson et al. (1992) J. Virol. 66(6):3633. Thus, adenoviral vectors containing the ADP gene may render the adenoviral vector more potent, making possible more effective treatment and/or a lower dosage requirement. The ADP may be expressed from its native location in E3 or at a location other than the native location (e.g. in the E1 region) or in both the native and a non-native location.

Accordingly, in one embodiment the invention provides adenovirus vectors in which an adenovirus gene is under transcriptional control of a first transcriptional regulatory element and a polynucleotide sequence encoding an ADP under control of a second transcriptional regulatory element. Preferably the adenovirus gene is essential for replication. The DNA sequence encoding ADP and the amino acid sequence of an ADP are publicly available. Briefly, an ADP coding sequence is obtained preferably from Ad2 (since this is the strain in which ADP has been more fully characterized) using techniques known in the art, such as PCR. Preferably, the Y leader (which is an important sequence for correct expression of late genes) is also obtained and ligated to the ADP coding sequence. The ADP coding sequence (with or without the Y leader) can then be introduced into the adenoviral genome, for example, in the E3 region (where the ADP coding sequence will be driven by the MLP). The ADP coding sequence could also be inserted in other locations of the adenovirus genome, such as the E4 region. In some embodiments, the ADP coding sequence is operably linked to a different TRE, e.g. a heterologous or native TRE. Adenovirus vectors comprising an ADP coding sequence may exhibit over expression of ADP.

Methods of packaging polynucleotides into adenovirus particles are known in the art and are also described in co-owned PCT PCT/US98/04080. The preferred packaging cells are those that have been designed to limit homologous recombination that could lead to wildtype adenoviral particles. Cells that may be used to produce the adenoviral particles of the invention include the human embryonic kidney cell line 293 (Graham et al., J Gen. Virol. 36:59-72 (1977)), the human embryonic retinoblast cell line PER.C6 (U.S. Pat. Nos. 5,994,128 and 6,033,908; Fallaux et al., Hum. Gene Ther. 9:1909-1917 (1998)), and the human cervical tumor-derived cell line HeLa-S3 (U.S. Patent Application No. 60/463,143).

The present invention contemplates the use of all adenoviral serotypes to construct the adenoviral vectors and virus particles according to the present invention. In one embodiment, the adenoviral nucleic acid backbone is derived from adenovirus serotype 2(Ad2), 5 (Ad5) or 35 (Ad35), although other serotype adenoviral vectors can be employed. Adenoviral stocks that can be employed according to the invention include any adenovirus serotype. A large number of adenovirus serotypes are currently available from American Type Culture Collection (ATCC, Manassas, Va.), and the invention includes any serotype of adenovirus available from any source. The adenoviruses that can be employed according to the invention may be of human or non-human origin. For instance, an adenovirus can be of subgroup A (e.g., serotypes 12, 18, 31), subgroup B (e.g., serotypes 3, 7, 11, 14, 16, 21, 34, 35), subgroup C (e.g., serotypes 1, 2, 5, 6), subgroup D (e.g., serotypes 8, 9, 10, 13, 15, 17, 19, 20, 22-30, 32, 33, 36-39, 42-47), subgroup E (serotype 4), subgroup F (serotype 40, 41), or any other adenoviral serotype. Throughout the specification reference is made to specific nucleotides in adenovirus type 5. One skilled in the art can determine the corresponding nucleotides in other serotypes and therefore construct similar adenoviral vectors in other adenovirus serotypes. Numerous examples of human and animal adenoviruses are available in the American Type Culture Collection, found e.g., at http://www.atcc.org/SearchCatalogs/CellBiology.cfm.

In one aspect the present invention provides a recombinant adenoviral vector comprising an adenoviral nucleic acid backbone, wherein said nucleic acid backbone comprises in sequential order: a left ITR, a termination signal sequence, a cancer specific PIN1 TRE of the invention that is operatively linked to a first gene essential for replication of the recombinant adenoviral vector, and a right ITR.

In another aspect, the present invention provides a recombinant adenoviral vector comprising an adenoviral nucleic acid backbone, wherein said nucleic acid backbone comprises in sequential order: a left ITR, a termination signal sequence, a PIN1 TRE of the invention that is operatively linked to a first gene essential for replication of the recombinant adenoviral vector, an adenoviral packaging signal, and a right ITR.

In another aspect, the present invention provides a recombinant adenoviral vector comprising an adenoviral nucleic acid backbone, wherein said nucleic acid backbone comprises in sequential order: a left ITR, an adenoviral packaging signal, a first TRE operatively linked to a first gene essential for replication of the recombinant adenoviral vector, a TRE operatively linked to a second gene essential for replication (wherein the first and second TREs are not the same), and a right ITR.

In yet another aspect, the present invention provides a recombinant adenoviral vector comprising an adenoviral nucleic acid backbone, wherein said nucleic acid backbone comprises in sequential order: a left ITR, an adenoviral packaging signal, a TRE operatively linked to a first gene essential for replication of the recombinant adenoviral vector, a second TRE operatively linked to a transgene and a right ITR.

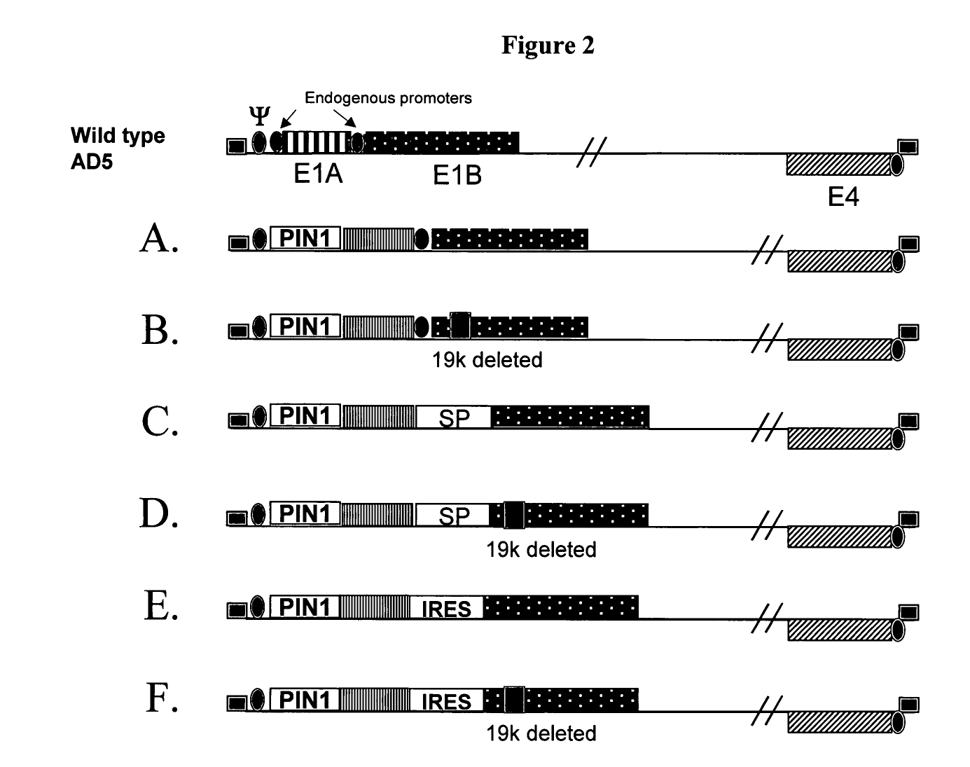

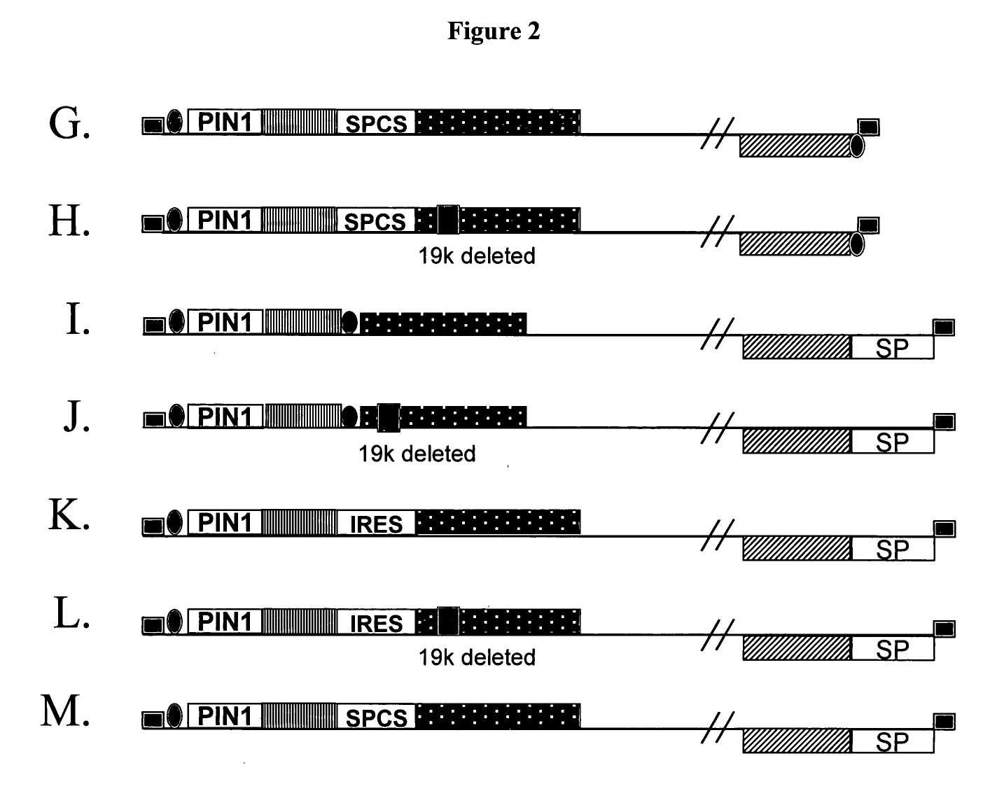

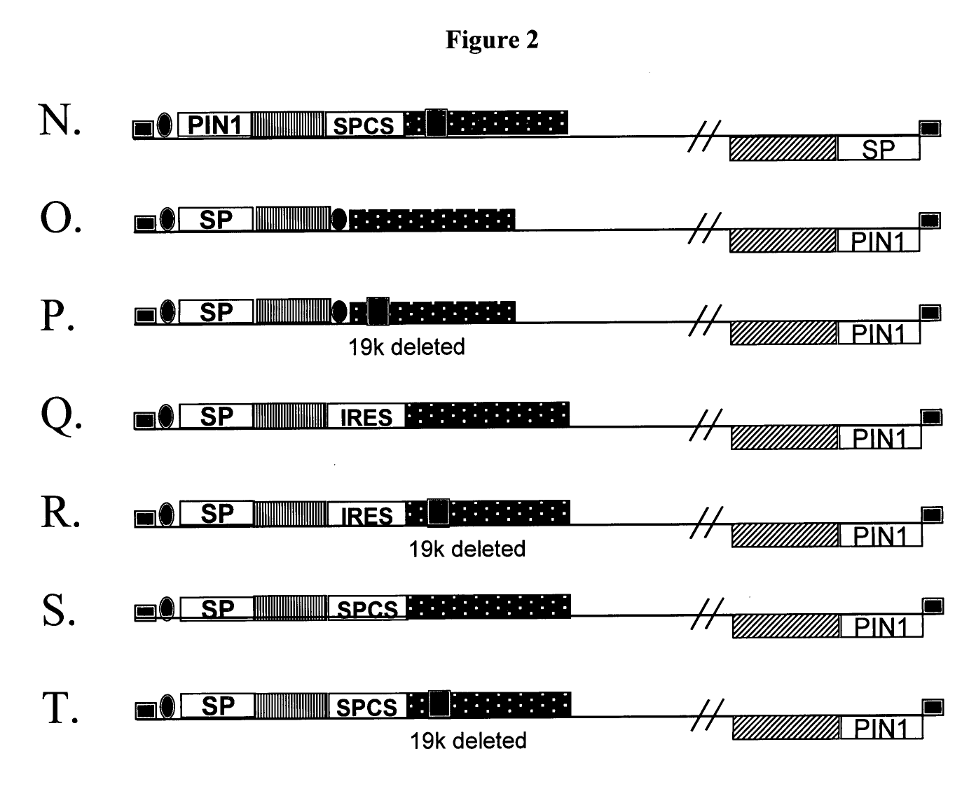

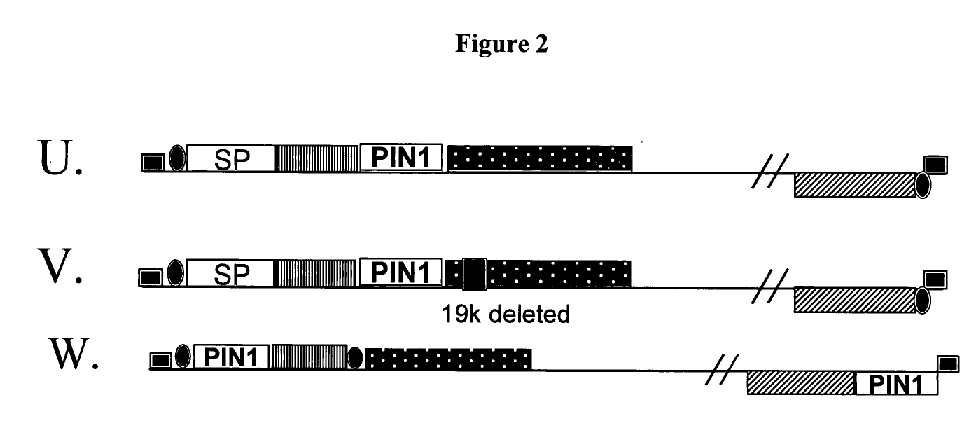

The first and second TREs may be cancer specific regulatory regions and may or may not be essentially the same. The vector may or may not have a termination signal sequence 5′ to the first cancer specific regulatory region and may or may not have a relocated packaging signal. In one embodiment, the first cancer specific regulatory region is a PIN1 TRE operatively linked to E1a and the second regulatory region is an hTERT TRE or an E2F-1 TRE operatively linked to E1b or E4. In another embodiment, the first cancer specific regulatory region is an hTERT TRE or an E2F-1 TRE operatively linked to E1a and the second cancer specific regulatory region is a PIN1 TRE operatively linked to E1b or E4. Exemplary vectors for use in practicing the inventions are illustrated in FIGS. 2B-W.

The recombinant adenoviral vectors of the invention are useful as therapeutics for treatment of cancer. As demonstrated herein, PIN1 expression is upregulated in tumor cells. Without wishing to be limited by theoretical considerations, the specific regulation of viral replication by a PIN1 TRE, which optionally may be shielded from read-through transcription by an upstream termination signal sequence, avoids toxicity that would occur if it replicated in non-target tissues, allowing for the favorable efficacy/toxicity profile.

In one embodiment, the recombinant viral vector of the invention comprises a termination signal sequence. A termination signal sequence may also be placed before (5′ to) any TRE in the vector. In one embodiment, the terminal signal sequence is placed before a heterologous TRE operatively linked to the E1b or E4 gene, e.g. an hTERT TRE.

In another embodiment, the recombinant viral vector further comprises a deletion upstream of the termination signal sequence, such as a deletion between nucleotides 103 and 551 of the adenoviral type 5 backbone or corresponding positions in other serotypes. A deletion in the packaging signal 5′ to the termination signal sequence may be such that the packaging signal becomes non-functional. In one embodiment, the deletion comprises a deletion 5′ to the termination signal sequence wherein the deletion spans at least the nucleotides 189 to 551. In another embodiment the deletion comprises a deletion 5′ to the termination signal sequence wherein the deletion spans at least nucleotides 103 to 551 (FIG. 2 of WO 02/067861 and WO 02/068627). In one embodiment, the packaging signal is located (i.e. re-inserted) downstream of the PIN1 TRE-linked gene essential for replication.

Additional Heterologous TREs

The vector may further comprise one or more additional heterologous TREs, which may or may not be cancer-specific. An adenovirus vector may further include an additional heterologous TRE, which may or may not be operably linked to the same gene(s) as a target cell-specific TRE. For example a TRE (such as a cell type-specific or cell status-specific TRE) may be juxtaposed to a different type of heterologous TRE. “Juxtaposed” means a target cell-specific TRE and second TRE transcriptionally control the same gene. For these embodiments, the target cell-specific TRE and the second TRE may be in any of a number of configurations, including, but not limited to, (a) next to each other (i.e., abutting); (b) both 5′ to the gene that is transcriptionally controlled (i.e., may have intervening sequences between them); (c) one TRE 5′ and the other TRE 3′ to the gene. The one or more additional heterologous TREs may be operably linked to an adenoviral gene essential for replication or a transgene, i.e., a therapeutic gene. In one aspect of the invention, the one or more additional TREs comprises a cell status TRE such as a “telomerase TRE” or “TERT TRE”, an “E2F TRE” or HRE TRE, described for example in WO 00/15820, a melanoma-specific TRE such as a MART-1 or TRP-1 TRE, described for example in U.S. Patent Publication No. 2003-0039633, a colon cancer specific regulatory sequence such as a PRL-3 transcriptional regulatory element (“PRL-3-TRE”) described for example in WO 2004/009790, a “plasminogen activator urokinase (uPA)” TRE (“uPA-TRE”), described for example in WO 98/39464, or an EBV-specific transcriptional regulatory element (TRE), described for example in WO 2004/042025.

As used herein, a TRE derived from a specific gene is referred to by the gene from which it was derived and is a polynucleotide sequence which regulates transcription of an operatively linked polynucleotide sequence in a host cell that expresses the gene. For example, as used herein, a “human glandular kallikrein transcriptional regulatory element”, or “hKLK2-TRE” is a polynucleotide sequence, preferably a DNA sequence, which increases transcription of an operatively linked polynucleotide sequence in a host cell that allows an hKLK2-TRE to function, such as a cell (preferably a mammalian cell, even more preferably a human cell) that expresses androgen receptor, such as a prostate cell. An hKLK2-TRE is thus responsive to the binding of androgen receptor and comprises at least a portion of an hKLK2 promoter and/or an hKLK2 enhancer (i.e., the ARE or androgen receptor binding site). Human glandular kallikrein enhancers and adenoviral vectors comprising the enhancer are described in WO99/06576, expressly incorporated by reference herein.

As used herein, a “probasin (PB) transcriptional regulatory element”, or “PB-TRE” is a polynucleotide sequence, preferably a DNA sequence, which selectively increases transcription of an operatively-linked polynucleotide sequence in a host cell that allows a PB-TRE to function, such as a cell (preferably a mammalian cell, more preferably a human cell, even more preferably a prostate cell) that expresses androgen receptor. A PB-TRE is thus responsive to the binding of androgen receptor and comprises at least a portion of a PB promoter and/or a PB enhancer (i.e., the ARE or androgen receptor binding site). Adenovirus vectors specific for cells expressing androgen are described in WO 98/39466, expressly incorporated by reference herein.

As used herein, a “prostate-specific antigen (PSA) transcriptional regulatory element”, or “PSA-TRE”, or “PSE-TRE” is a polynucleotide sequence, preferably a DNA sequence, which selectively increases transcription of an operatively linked polynucleotide sequence in a host cell that allows a PSA-TRE to function, such as a cell (preferably a mammalian cell, more preferably a human cell, even more preferably a prostate cell) that expresses androgen receptor. A PSA-TRE is thus responsive to the binding of androgen receptor and comprises at least a portion of a PSA promoter and/or a PSA enhancer (i.e., the ARE or androgen receptor binding site). A tissue-specific enhancer active in prostate and used in adenoviral vectors is described in WO 95/19434 and WO 97/01358, each of which is expressly incorporated by reference herein.

As used herein, a “carcinoembryonic antigen (CEA) transcriptional regulatory element”, or “CEA-TRE” is a polynucleotide sequence, preferably a DNA sequence, which selectively increases transcription of an operatively linked polynucleotide sequence in a host cell that allows a CEA-TRE to function, such as a cell (preferably a mammalian cell, even more preferably a human cell) that expresses CEA. The CEA-TRE is responsive to transcription factors and/or co-factor(s) associated with CEA-producing cells and comprises at least a portion of the CEA promoter and/or enhancer. Adenovirus vectors specific for cells expressing carcinoembryonic antigen are described in WO 98/39467, expressly incorporated by reference herein.

As used herein, an “alpha-fetoprotein (AFP) transcriptional regulatory element”, or “AFP-TRE” is a polynucleotide sequence, preferably a DNA sequence, which selectively increases transcription (of an operatively linked polynucleotide sequence) in a host cell that allows an AFP-TRE to function, such as a cell (preferably a mammalian cell, even more preferably a human cell) that expresses AFP. The AFP-TRE is responsive to transcription factors and/or co-factor(s) associated with AFP-producing cells and comprises at least a portion of the AFP promoter and/or enhancer. Adenovirus vectors specific for cells expressing alpha-fetoprotein are described in WO 98/39465, expressly incorporated by reference herein.

As used herein, “a mucin gene (MUC) transcriptional regulatory element”, or “MUC1-TRE” is a polynucleotide sequence, preferably a DNA sequence, which selectively increases transcription (of an operatively-linked polynucleotide sequence) in a host cell that allows a MUC1-TRE to function, such as a cell (preferably a mammalian cell, even more preferably a human cell) that expresses MUC1. The MUC1-TRE is responsive to transcription factors and/or co-factor(s) associated with MUC1-producing cells and comprises at least a portion of the MUC1 promoter and/or enhancer.

In yet another aspect, the invention provides adenoviral vectors comprising a “telomerase promoter” or “TERT promoter” operatively linked to a gene essential for adenovirus replication or a transgene. The term “telomerase TRE” or “TERT TRE” as used herein refers to a native TERT TRE (e.g. TERT promoter) and functional fragments, mutations and derivatives thereof. The TERT promoter does not have to be the full-length or wild type promoter. One skilled in the art knows how to derive fragments from a TERT TRE, e.g. a TERT promoter, and test them for the desired selectivity. A TERT promoter fragment of the present invention has promoter activity selective for tumor cells, i.e. drives tumor selective expression of an operatively linked coding sequence. In one embodiment, the TERT TRE of the invention is a mammalian TERT promoter. In another embodiment, the mammalian TERT TRE is a human TERT (hTERT) promoter. See, e.g., WO 98/14593 and WO 00/46355 for exemplary TERT promoters that find utility in the compositions and methods of the present invention. In one embodiment, a TERT TRE according to the present invention comprises the sequence shown in SEQ ID NO:4 or is a full-length complement that hybridizes to the sequence shown in SEQ ID NO:4 under stringent conditions.

The protein urokinase plasminogen activator (uPA) and its cell surface receptor, urokinase plasminogen activator receptor (uPAR), are expressed in many of the most frequently-occurring neoplasms and appear to represent important proteins in cancer metastasis. Both proteins are implicated in breast, colon, prostate, liver, renal, lung and ovarian cancer. Sequence elements that regulate uPA and uPAR transcription have been extensively studied. Riccio et al. (1985) Nucleic Acids Res. 13:2759-2771; Cannio et al. (1991) Nucleic Acids Res. 19:2303-2308. See also, WO 98/39464.

As used herein, a “urothelial cell-specific transcriptional response element” or “urothelial cell-specific TRE” is polynucleotide sequence, preferably a DNA sequence, which increases transcription of an operatively linked polynucleotide sequence in a host cell that allows a urothelial-specific TRE to function, i.e., a target cell. A variety of urothelial cell-specific TREs are known, are responsive to cellular proteins (transcription factors and/or co-factor(s)) associated with urothelial cells, and comprise at least a portion of a urothelial-specific promoter and/or a urothelial-specific enhancer. Exemplary urothelial cell specific transcriptional regulatory sequences include a human or rodent uroplakin (UP), e.g., UPI, UPII, UPIII and the like. Human urothelial cell specific uroplakin transcriptional regulatory sequences and adenoviral vectors comprising the same are described in WO 01/72994, expressly incorporated by reference herein.

As used herein, a “melanocyte cell-specific transcriptional response element”, or “melanocyte cell-specific TRE” is a polynucleotide sequence, preferably a DNA sequence, which increases transcription of an operatively linked polynucleotide sequence in a host cell that allows a melanocyte-specific TRE to function, i.e., a target cell. A variety of melanocyte cell-specific TREs are known, are responsive to cellular proteins (transcription factors and/or co-factor(s)) associated with melanocyte cells, and comprise at least a portion of a melanocyte-specific promoter and/or a melanocyte-specific enhancer. Methods are described herein for measuring the activity of a melanocyte cell-specific TRE and thus for determining whether a given cell allows a melanocyte cell-specific TRE to function. Examples of a melanocyte-specific TRE for use in practicing the invention include but are not limited to a TRE derived from the 5′ flanking region of a tyrosinase gene, a tyrosinase related protein-1 (TRP-1) gene, a TRE derived from the 5′-flanking region of a tyrosinase related protein-2 (TRP-2) gene, a TRE derived from the 5′ flanking region of a MART-1 gene or a TRE derived from the 5′-flanking region of a gene which is aberrantly expressed in melanoma.

In another aspect, the invention provides adenoviral vectors comprising a metastatic colon cancer specific TRE derived from a PRL-3 gene operatively linked to a gene essential for adenovirus replication or a transgene. As used herein, a “metastatic colon cancer specific TRE derived from a PRL-3 gene” or a “PRL-3 TRE” is a polynucleotide sequence, preferably a DNA sequence, which selectively increases transcription of an operatively linked polynucleotide sequence in a host cell that allows a PRL-3 TRE to function, such as a cell (preferably a mammalian cell, more preferably a human cell, even more preferably a metastatic colon cancer cell). The metastatic colon cancer-specific TRE may comprise one or more regulatory sequences, e.g. enhancers, promoters, transcription factor binding sites and the like, which may be derived from the same or different genes. In one preferred aspect, the PRL-3 TRE comprises a PRL-3 promoter. One preferred PRL-3 TRE is derived from the 0.6 kb sequence upstream of the translational start codon for the PRL-3 gene, described in WO 04/009790, expressly incorporated by reference herein. Examples of PRL-3 TREs include, but are not limited to the sequences presented as a 0.6 kb and 1 kb sequence upstream of the translational start codon for the PRL-3 gene (identified as SEQ ID NO:1 and SEQ ID NO:2 in WO 2004/009790. The PRL-3 protein tyrosine phosphatase gene is specifically expressed at a high level in metastatic colon cancers (Saha et al. (2001) Science 294:1343).

In another aspect, the invention provides adenoviral vectors comprising a liver cancer specific TREs derived from the CRG-L2 gene operatively linked to a gene essential for adenovirus replication or a transgene. As used herein, a “liver cancer specific TREs derived from the CRG-L2 gene” or a “CRG-L2 TRE” is a polynucleotide sequence, preferably a DNA sequence, which selectively increases transcription of an operatively linked polynucleotide sequence in a host cell that allows a CRG-L2 to function, such as a cell (preferably a mammalian cell, more preferably a human cell, even more preferably a hepatocellular carcinoma cell). The hepatocellular carcinoma specific TRE may comprise one or more regulatory sequences, e.g. enhancers, promoters, transcription factor binding sites and the like, which may be derived from the same or different genes. In one preferred aspect, the CRG-L2 TRE may be derived from the 0.8 kb sequence upstream of the translational start codon for the CRG-L2 gene, or from a 0.7 kb sequence contained within the 0.8 kb sequence (residues 119-803); or from an EcoRI to NcoI fragment derived from the 0.8 kb sequence, as described in U.S. application Ser. No. 10/947,812, expressly incorporated by reference herein.

In another aspect, the invention provides adenoviral vectors comprising an EBV-specific transcriptional regulatory element (TRE) operatively linked to a gene essential for adenovirus replication or a transgene. In one aspect, the EBV specific TRE is derived from a sequence upstream of the translational start codon for the LMP1, LMP2A or LMP2B genes, as further described in U.S. application Ser. No.10/698,160, expressly incorporated by reference herein. The EBV-specific TRE may comprise one or more regulatory sequences, e.g. enhancers, promoters, transcription factor binding sites and the like, which may be derived from the same or different genes.

In yet another aspect, the invention provides adenoviral vectors comprising a hypoxia-responsive element (“HRE”) operatively linked to a gene essential for adenovirus replication or a transgene. HRE is a transcriptional regulatory element comprising a binding site for the transcriptional complex HIF-1, or hypoxia inducible factor-1, which interacts with a sequence in the regulatory regions of several genes, including vascular endothelial growth factor, and several genes encoding glycolytic enzymes, including enolase-1. Accordingly, in one embodiment, an adenovirus vector comprises an adenovirus gene, preferably an adenoviral gene essential for replication, under transcriptional control of a cell status-specific TRE such as a HRE, as further described in WO 00/15820, expressly incorporated by reference herein.

The term “E2F TRE” as used herein refers to a native E2F TRE (e.g. an E2F promoter) and functional fragments, mutations and derivatives thereof. The E2F TRE does not have to be a full-length or wild type E2F promoter. One skilled in the art knows how to derive fragments from an E2F promoter and test them for the desired selectivity. An E2F promoter fragment of the present invention has promoter activity selective for tumor cells, i.e. drives tumor selective expression of an operatively linked coding sequence. The term “tumor selective promoter activity” as used herein means that the promoter activity of a promoter fragment of the present invention in tumor cells is higher than in non-tumor cell types. A number of examples of E2F TREs are known in the art. See, e.g., Parr et al. Nature Medicine 1997:3(10) 1145-1149, WO 02/067861, U.S. 20010053352 and WO 98/13508. In another embodiment, an E2F promoter according to the present invention comprises the sequence shown in SEQ ID NO:5 or is a full-length complement that hybridizes to the sequence shown in SEQ ID NO:5 under stringent conditions.

The adenovirus vectors of the invention replicate preferentially in carcinoma cells. Replication preference is indicated by comparing the level of replication (e.g., cell killing and/or titer) in carcinoma cells to the level of replication in non-carcinoma cells, normal or control cells. Comparison of the adenovirus titer of a carcinoma cell to the titer of a TRE inactive cell type provides a key indication that the overall replication preference is enhanced due to the replication in target cells as well as depressed replication in non-target cells. Runaway infection is prevented due to the cell-specific requirements for viral replication. Without wishing to be bound by any particular theory, production of adenovirus proteins can serve to activate and/or stimulate the immune system, either generally or specifically toward target cells producing adenoviral proteins which can be an important consideration in the cancer context, where individuals are often moderately to severely immunocompromised.

In one embodiment of a recombinant viral vector of the invention, the PIN1 TRE is a human PIN1 TRE.

In one embodiment of a recombinant viral vector of the invention, the coding sequence of a gene essential for replication is selected from the group consisting of E1A, E1B, E2A, E2B and E4 coding sequences. In one embodiment, the PIN1 TRE is operatively linked to one of either the ELA, E1B or E4 coding sequence. In another embodiment, the vector further comprises an additional heterologous TRE operatively linked to an ELA, E1B or E4 coding sequence. In one embodiment, the hTERT TRE may comprise SEQ ID NO:2, 3 or 4. The “E2F TRE” may comprise SEQ ID NO:5. In one embodiment, the PIN1 TRE is operatively linked to the E1A coding sequence and a different TRE is operatively linked to the E1B or E4 coding sequence.

In another embodiment of a recombinant viral vector of the invention, the nucleic acid backbone further comprises a termination signal sequence upstream of the PIN1 TRE operatively linked to the coding sequence of a gene essential for replication of the recombinant viral vector. In one embodiment, the termination signal sequence is the SV40 early polyadenylation signal sequence. In another embodiment, the vector further comprises a deletion upstream of the termination signal sequence. For example, the vector may comprise a deletion between nucleotides corresponding to nucleotides 103 and 551 of the adenoviral type 5 backbone (e.g. see WO 02/68627. Vectors based on other adenovirus serotypes may have the same corresponding nucleotides deleted.

In one embodiment, the adenoviral vector comprises a transgene which is inserted in the E3 region of the adenoviral nucleic acid backbone. For example, a transgene may be inserted in place of the 19 kD or 14.7 kD E3 gene. Any of a number of transgenes known in the art may be included in an adenovirus vector of the invention examples of which are described herein. In one aspect of this embodiment, the transgene encodes an immunostimulatory protein, e.g. a cytokine such as GM-CSF. In yet another aspect, the transgene encodes an anti-angiogenic protein. In still another aspect, the transgene is a suicide gene. In yet another aspect, the transgene is ADP.

IRESs and Self-Processing Cleavage Sites (SPCSs)

The adenovirus vectors of the present invention may comprise an intergenic IRES element(s) or a coding sequence for a self-processing cleavage site (SPCS) which links the translation of two or more genes. The use of an IRES or a SPCS rather than a second TRE provides additional space in the vector for an additional gene(s) such as a therapeutic gene or longer TREs. Accordingly, in one aspect of the invention, the viral vectors disclosed herein comprise at least one IRES or code for a SPCS within a multicistronic transcript, wherein production of the multicistronic transcript is regulated by a heterologous, target cell-specific TRE (e.g. a PIN1 TRE). For adenovirus vectors comprising a second gene under control of an IRES or SPCS, it is preferred that the endogenous promoter of the second be deleted so that the endogenous promoter does not interfere with transcription of the second gene. It is preferred that the second gene be in frame with the IRES if the IRES contains an initiation codon and SPCS coding sequence. If an initiation codon, such as ATG, is present in the IRES, it is preferred that the initiation codon of the second gene is removed and that the IRES and the second gene are in frame. In one embodiment, the adenovirus vectors comprise the adenovirus essential genes, E1A and E1B genes, under the transcriptional control of a PIN1 TRE, and an IRES or SPCS coding sequence introduced between E1A and E1B. Thus, both E1A and E1B are under common transcriptional control, and translation of E1B coding region is obtained by virtue of the presence of the IRES or SPCS. In one embodiment, E1A has its endogenous promoter deleted. In another embodiment, E1A has an endogenous enhancer deleted and in yet an additional embodiment, E1A has its endogenous promoter deleted and an E1A enhancer deleted. In another embodiment, E1B has its endogenous promoter deleted. In yet further embodiments, E1B has a deletion of part or all of the 19-kDa region of E1B.

Insertion of an IRES or SPCS into a vector is accomplished by methods and techniques that are known in the art and described herein supra, including but not limited to, restriction enzyme digestion, ligation, and PCR. A DNA copy of an IRES or SPCS coding sequence can be obtained by chemical synthesis, or by making a cDNA copy of, for example, a picornavirus IRES. See, for example, Duke et al. (1995) J. Virol. 66(3):1602-9) for a description of the EMCV IRES and Huez et al. (1998), Mol. Cell. Biol. 18(11):6178-90) for a description of the VEGF IRES. SPCS coding sequences and amino acid sequences are further described herein. The sequence is inserted into a vector genome at a site such that it lies upstream of a 5′-distal coding region in a multicistronic mRNA. IRES sequences of cardioviruses and certain aphthoviruses contain an AUG codon at the 3′ end of the IRES that serves as both a ribosome entry site and as a translation initiation site. Accordingly, this type of IRES is introduced into a vector so as to replace the translation initiation codon of the protein whose translation it regulates. However, in an IRES of the entero/rhinovirus class, the AUG at the 3′ end of the IRES is used for ribosome entry only, and translation is initiated at the next downstream AUG codon. Accordingly, if an entero/rhinovirus IRES is used in a vector for translational regulation of a downstream coding region, the AUG (or other translation initiation codon) of the downstream gene is retained in the vector construct.

In another aspect of the invention a “self-processing cleavage site” (e.g. 2A-like sequence) is utilized to express two polypeptides from one mRNA. A “self-processing cleavage site” or “self-processing cleavage sequence” is defined as a DNA or amino acid sequence, wherein upon translation, rapid intramolecular (cis) cleavage of a polypeptide comprising the self-processing cleavage site occurs to result in expression of discrete mature protein or polypeptide products. Such a “self-processing cleavage site”, may also be referred to as a post-translational or co-translational processing cleavage site, exemplified herein by a 2A site, sequence or domain. As used herein, a “self-processing peptide” is defined herein as the peptide expression product of the DNA sequence that encodes a self-processing cleavage site or sequence, which upon translation, mediates rapid intramolecular (cis) cleavage of a protein or polypeptide comprising the self-processing cleavage site to yield discrete mature protein or polypeptide products. It has been reported that a 2A site, sequence or domain demonstrates a translational effect by modifying the activity of the ribosome to promote hydrolysis of an ester linkage, thereby releasing the polypeptide from the translational complex in a manner that allows the synthesis of a discrete downstream translation product to proceed (Donnelly et al. J Gen Virol. May 2001;82(Pt 5):1013-25). Alternatively, it has also been reported that a 2A site, sequence or domain demonstrates “auto-proteolysis” or “cleavage” by cleaving its own C-terminus in cis to produce primary cleavage products (Furler; Palmenberg, Ann. Rev. Microbiol. 44:603-623 (1990)).

Although the mechanism is not part of the invention, the activity of a 2A-like sequence may involve ribosomal skipping between codons which prevents formation of peptide bonds (de Felipe et al., Human Gene Therapy 11:1921-1931 (2000); Donnelly et al., J. Gen. Virol. 82:1013-1025 (2001); Donnelly et al. J Gen Virol. May 2001;82(Pt 5):1027-41); Szymczak et al., Nature Biotechnology 22:589-594 and 760 (2004), although it has been considered that the domain acts more like an autolytic enzyme (Ryan et al., Virol. 173:35-45 (1989)). Studies in which the Foot and Mouth Disease Virus (FMDV) 2A coding region was cloned into expression vectors and transfected into target cells showed FMDV 2A cleavage of artificial reporter polyproteins in wheat-germ lysate and transgenic tobacco plants (Halpin et al., U.S. Pat. No. 5,846,767; 1998 and Halpin et al., Plant J 17:453-459, 1999); Hs 683 human glioma cell line (de Felipe et al., Gene Therapy 6:198-208, 1999); rabbit reticulocyte lysate and human HTK-143 cells (Ryan et al., EMBO J. 13:928-933 (1994)); and insect cells (Roosien et al., J. Gen. Virol. 71:1703-1711, 1990). The FMDV 2A-mediated cleavage of a heterologous polyprotein has been shown for IL-12 (p40/p35 heterodimer; Chaplin et al., J. Interferon Cytokine Res. 19:235-241, 1999). The reference demonstrates that in transfected COS-7 cells, FMDV 2A mediated the cleavage of a p40-2A-p35 polyprotein into biologically functional subunits p40 and p35 having activities associated with IL-12.

The FMDV 2A sequence has been incorporated into retroviral vectors, alone or combined with different IRES sequences to construct bicistronic, tricistronic and tetracistronic vectors. The efficiency of 2A-mediated gene expression in animals was demonstrated by Furler et al. (Gene Ther. June 2001;8(11):864-73) using recombinant adeno-associated viral (AAV) vectors encoding α-synuclein and EGFP or Cu/Zn superoxide dismutase (SOD-1) and EGFP linked via the FMDV 2A sequence. EGFP and a-synuclein were expressed at substantially higher levels from vectors which included a 2A sequence relative to corresponding IRES-based vectors, while SOD-1 was expressed at comparable or slightly higher levels. Furler also demonstrated that the 2A sequence results in bicistronic gene expression in vivo after injection of 2A-containing AAV vectors into rat substantia nigra. Syzmczak et al. (Nature Biotechnology 22:589-594&760 (2004)) describe a retroviral vector with four coding regions linked with three 2A sequences.

For the present invention, the DNA sequence encoding a self-processing cleavage site is exemplified by viral sequences derived from a picornavirus, including but not limited to an entero-, rhino-, cardio-, aphtho- or Foot-and-Mouth Disease Virus (FMDV). In one embodiment, the self-processing cleavage site coding sequence is derived from a FMDV. Self-processing cleavage sites include, but are not limited to, 2A and 2A-like sites, sequences or domains (Donnelly et al., J. Gen. Virol. 82:1027-1041 (2001)).

FMDV 2A is a polyprotein region, which functions in the FMDV genome to direct a single cleavage at its own C-terminus, thus functioning in cis. The FMDV 2A domain is typically reported to be about nineteen amino acids in length ((LLNFDLLKLAGDVESNPGP (SEQ ID NO:6); TLNFDLLKLAGDVESNPGP (SEQ ID NO:7); Ryan et al., J. Gen. Virol. 72:2727-2732 (1991)), however oligopeptides of as few as fourteen amino acid residues ((LLKLAGDVESNPGP (SEQ ID NO:8)) have also been shown to mediate cleavage at the 2A C-terminus in a fashion similar to its role in the native FMDV polyprotein processing.

Variations of the 2A sequence have been studied for their ability to mediate efficient processing of polyproteins (Donnelly et al., J. Gen. Virol. 82:1027-1041 (2001)). Homologues and variant 2A sequences are included within the scope of the invention and include, but are not limited to, the sequences presented in Table 1, below:

TABLE 1

Exemplary 2A and 2A-like Sequences
(Self-processing cleavage sites)

| | |
|---|---|
| LLNFDLLKLAGDVESNPGP | (SEQ ID NO:6) |
| TLNFDLLKLAGDVESNPGP; | (SEQ ID NO:7) |
| LLKLAGDVESNPGP | (SEQ ID NO:8) |
| NFDLLKLAGDVESNPGP | (SEQ ID NO:9) |
| QLLNFDLLKLAGDVESNPGP | (SEQ ID NO:10) |
| APVKQTLNFDLLKLAGDVESNPGP. | (SEQ IP NO:11) |
| VTELLYRMKRAETYCPRPLLAIHPTEARHKQKIVAP | (SEQ ID NO:12) |
| VKQTLNFDLLKLAGDVESNPGP | |
| LLAIHPTEARHKQKIVAPVKQTLNFDLLKLAGDVES | (SEQ ID NO:13) |
| NPGP | |
| EARHKQKIVAPVKQTLNFDLLKLAGDVESNPGP | (SEQ ID NO:14) |
| NFDLLKLAGDVESNPGPFF | (SEQ ID NO:15) |
| GIFNAHYAGYFADLLIHDIETNPGP | (SEQ ID NO:16) |
| RIFNAHYAGYFADLLIHDIETNPGP | (SEQ ID NO:17) |
| HVFETHYAGYFADLLIHDVETNPGP | (SEQ ID NO:18) |
| KAVRGYHADYYKQRLIHDVEMNPGP | (SEQ ID NO:19) |
| RAVRAYHADYYKQRLIHDVEMNPGP | (SEQ ID NO:20) |
| KAVRGYHADYYRQRLIHDVETNPGP | (SEQ ID NO:21) |
| LTNFDLLKLAGDVESNPGP | (SEQ ID NO:22) |
| LLNFDLLKLAGDMESNPGP | (SEQ ID NO:23) |
| MCNFDLLKLAGDVESNPGP | (SEQ ID NO:24) |
| CTNYALLKLAGDVESNPGP | (SEQ ID NO:25) |
| GATNFSLLKLAGDVELNPGP | (SEQ ID NO:26) |
| GPGATNFSLLKQAGDVEENPGP | (SEQ ID NO:27) |
| EAARQMLLLLSGDVETNPGP | (SEQ ID NO:28) |
| FLRKRTQLLMSGDVESNPGP | (SEQ ID NO:29) |
| GSWTDILLLLSGDVETNPGP | (SEQ ID NO:30) |
| RAEGRGSLLTCGDVEENPGP | (SEQ ID NO:31) |
| TRAEIEDELIRAGIESNPGP | (SEQ ID NO:32) |
| SKFQIDRILISGDIELNPGP | (SEQ ID NO:33) |
| AKFQIDKILISGDVELNPGP | (SEQ ID NO:34) |
| SKFQIDKILISGDIELNPGP | (SEQ ID NO:35) |
| SSIIRTKMLVSGDVEENPGP | (SEQ ID NO:36) |
| CDAQRQKLLLSGDIEQNPGP | (SEQ ID NO:37) |

In one embodiment, the FMDV 2A sequence included in a vector according to the invention encodes amino acid residues comprising LLNFDLLKLAGDVESNPGP (SEQ ID NO:6). Alternatively, a vector according to the invention may encode amino acid residues for other 2A-like regions as discussed in Donnelly et al., J. Gen. Virol. 82:1027-1041 (2001) and including, but not limited to, a 2A-like domain from picornavirus, insect virus, Type C rotavirus, trypanosome repeated sequences or the bacterium, Thermatoga maritima.

The invention contemplates the use of nucleic acid sequence variants that encode a self-processing cleavage site, such as a 2A or 2A-like polypeptide, and nucleic acid coding sequences that have a different codon for one or more of the amino acids relative to that of the parent (native) nucleotide. Such variants are specifically contemplated and encompassed by the present invention. Sequence variants of self-processing cleavage peptides and polypeptides are included within the scope of the invention as well.

In accordance with the present invention, also encompassed are sequence variants which encode self-processing cleavage polypeptides, wherein the self-processing cleavage polypeptides themselves have 80, 85, 88, 89, 90, 91, 92, 93, 94, 95, 96, 97, 98, 99% or more sequence identity to the native sequence.

In one embodiment of the invention, a self-processing cleavage sequence (e.g. 2A or 2A-like sequence) is operatively linked to an adenovirus protein coding region and a transgene. The adenovirus protein CDS may be upstream of the self-processing cleavage site, with the transgene being downstream. Alternatively, the transgene CDS may be upstream of the self-processing cleavage site, with the adenovirus protein CDS being downstream.

Multiple CDSs may be linked with self-processing cleavage sites. In one embodiment, an Ad CDS is operatively linked by a self-processing cleavage site to a first transgene and said first transgene is operatively linked by a self-processing cleavage site to a second transgene. In one embodiment, the first and second transgenes encodes for the same or different proteins. In the case of the same proteins, it is advantageous that the coding sequence of one of the transgenes be “recoded”. In other words, use different codons to code for the same amino acids. This is done to reduce the amount of homology between the two transgenes at the DNA level, thus reducing or eliminating homologous recombination between the two transgenes. Other embodiments include two Ad CDSs operatively linked by a self-processing cleavage site. This may accompany a deletion of adenoviral sequence. For example, two adenoviral CDSs that are located in the same leader region and are adjacent to each other may be operatively linked by a self-processing cleavage site and a portion or all of the intervening Ad sequence may be deleted as long the deletion does not disrupt other sequences or elements necessary for viral vector production, being especially mindful of the complementary strand. The deleted portion may be 1-5 nucleotides, 6-15 nucleotides, 16-25 nucleotides, 26-35 nucleotides, 36-40 nucleotides, or greater than 40 nucleotides.

In one embodiment, a first transgene CDS is operatively linked by a first self-processing cleavage site to an Ad CDS and the Ad CDS is operatively linked by a second self-processing cleavage site to a second transgene. Other embodiments include various combinations of Ad CDSs, and both Ad CDSs and transgene CDSs operatively linked with IRES and/or self-processing peptide sequences.

Also, multiple transgenes may be expressed by operatively linking them via self-processing cleavage site(s). The invention contemplates 2, 3, 4, 5 or more transgenes linked by self-processing cleavage sites. The self-processing cleavage sites may all be the same sequence or derived from the same source or may all be different sequences or derived from different sources.

When using multiple self-processing peptide sequences in a vector, it is preferable that the self-processing peptide sequences have minimal or no homology at the DNA level to reduce the frequency of homologous recombination. For example, the self-processing peptide sequences may be derived form different sources wherein the multiple coding sequences for self-processing peptide sequences have minimal or no homology. In another embodiment, a coding sequence for a self-processing peptide sequence is recoded. In other words, use different codons to code for the same amino acids of the self-processing peptide sequence. This is done to reduce the amount of homology between the two or more coding sequences for the self-processing peptide sequences, thus reducing or eliminating homologous recombination between the two transgenes.

A self-processing peptide sequence is operatively linked to a CDS when the sequence encoding the self-processing peptide sequence is inserted in frame with the upstream and downstream CDS.

Removal of Self-Processing Peptide Sequences.

One concern associated with the use of self-processing peptides, such as a 2A or 2A-like sequence is that the C terminus of the expressed polypeptide contains amino acids derived from the self-processing peptide, i.e. 2A-derived amino acid residues. These amino acid residues are “foreign” to the host and may elicit an immune response. when the recombinant protein is expressed in vivo or delivered in vivo following in vitro or ex vivo expression. In addition, if not removed, self-processing peptide-derived amino acid residues may interfere with protein function and/or alter protein conformation, resulting in a less than optimal expression level and/or reduced biological activity of the recombinant protein. In other words, depending on the application it may be advantageous that the resulting proteins not contain all of the 2A-derived amino acid residues.

The invention includes vectors, engineered such that an additional proteolytic cleavage site is provided between a first protein or polypeptide coding sequence (the first or 5′ ORF) and the self processing cleavage site as a means for removal of self processing cleavage site derived amino acid residues that are present in the expressed protein product.

Examples of additional proteolytic cleavage sites are furin cleavage sites with the consensus sequence RXK(R)R (SEQ ID NO:38), which can be cleaved by endogenous subtilisin-like proteases, such as furin and other serine proteases. Others have demonstrated that self processing 2A amino acid residues at the C terminus of a first expressed protein can be efficiently removed by introducing a furin cleavage site RAKR (SEQ ID NO:39) between the first polypeptide and a self processing 2A sequence. In addition, use of a plasmid containing a 2A sequence and a furin cleavage site adjacent to the 2A sequence was shown to result in a higher level of protein expression than a plasmid containing the 2A sequence alone. This improvement provides a further advantage in that when 2A amino acid residues are removed from the C-terminus of the protein, longer 2A- or 2A like sequences or other self-processing sequences can be used, as described in U.S. application Ser. No. 10/831304, expressly incorporated by reference herein.

It is often advantageous to produce therapeutic proteins, polypeptides, fragments or analogues thereof with fully human characteristics. These reagents avoid the undesired immune responses induced by proteins, polypeptides, fragments or analogues thereof originating from different species. To address possible host immune responses to amino acid residues derived from self-processing peptides, the coding sequence for a proteolytic cleavage site may be inserted (using standard methodology known in the art) between the coding sequence for a first protein and the coding sequence for a self-processing peptide so as to remove the self-processing peptide sequence from the expressed protein or polypeptide. This finds particular utility in therapeutic and diagnostic proteins and polypeptides for use in vivo.

Any additional proteolytic cleavage site known in the art that can be expressed using recombinant DNA technology may be employed in practicing the invention. Exemplary additional proteolytic cleavage sites which can be inserted between a polypeptide or protein coding sequence and a self processing cleavage sequence include, but are not limited to a:
a). Furin consensus sequence or site: RXK(R)R (SEQ ID. NO:38);
b). Factor Xa cleavage sequence or site: IE(D)GR (SEQ ID. NO:40);
c). Signal peptidase I cleavage sequence or site: e.g., LAGFATVAQA (SEQ ID. NO:41); and
d). Thrombin cleavage sequence or site: LVPRGS (SEQ ID. NO:42).
e). Adenoviral consensus protease sequence or site (M,L,I)XGG/X (SEQ ID NO:43) and (M,L,I)XGX/G (SEQ ID NO:44) see Webster et al. J Gen Virol 70:3215-3223 (1989); Weber, Curr Top Microbiol Immunol 199I:227-235 (1995) and Balakirev et al. J of Virol 76:6323-6331 (2002)

As set forth above, and shown in the Examples, when a furin cleavage site sequence, e.g., RAKR, is inserted between the first protein and the 2A sequence, the 2A residues are removed from the C-terminus of the first protein. However, mass spectrum data indicates that the C-terminus of the first protein expressed from the RAKR-2A construct contains two additional amino acid residues, RA, derived from the furin cleavage site RAKR.

In one embodiment, the invention provides a method for removal of residual amino acids and a composition for expression of the same. A number of novel constructs have been designed that provide for removal of these additional amino acids from the C-terminus of the protein. Furin cleavage occurs at the C-terminus of the cleavage site, which has the consensus sequence RXR(K)R, where X is any amino acid. In one aspect, the invention provides a means for removal of the newly exposed basic amino acid residues R or K from the C-terminus of the protein by use of an enzyme selected from a group of enzymes called carboxypeptidases (CPs), which include, but not limited to, carboxypeptidase D, E and H (CPD, CPE, CPH). Since CPs are able to remove basic amino acid residues at the C-terminus of a protein, all amino acid resides derived from a furin cleavage site which contain exclusively basic amino acids R or K, such as RKKR, RKRR, RRRR, etc, can be removed by a CP.

In the case of an adenovirus protease sequence or site, the invention is not meant to be limited to the consensus sequences provided above. The invention contemplates the use of any adenoviral protease. In one embodiment, the adenoviral protease is from the same adenovirus serotype as from which the adenoviral vector genome is derived.

Transgenes

To further enhance therapeutic efficacy, the vectors of the invention may include one or more transgenes that have a therapeutic effect, such as enhancing cytotoxicity so as to eliminate unwanted target cells. The transgene may be under the transcriptional control of a cancer-specific TRE, e.g. a PIN1 TRE. The transgene may be regulated independently of the adenovirus gene regulation, e.g. having separate promoters, which may be the same or different, or may be coordinately regulated, e.g. having a single promoter in conjunction with an IRES or a self-processing cleavage sequence, such as a 2A sequence.

In this way, various genetic capabilities may be introduced into target cells, particularly cancer cells. The vector may comprise a heterologous transgene encoding a therapeutic gene product under the control of a constitutive or inducible promoter. Numerous examples of constitutive and inducible promoters are known in the art and routinely employed in transgene expression in the context of viral or non-viral vectors. In this way, various genetic capabilities may be introduced into target cells. For example, in certain instances, it may be desirable to enhance the degree of therapeutic efficacy by enhancing the rate of cytotoxic activity. This could be accomplished by coupling the cancer cell-specific TRE activity with expression of, one or more metabolic enzymes such as HSV-tk, nitroreductase, cytochrome P450 or cytosine deaminase (CD) which render cells capable of metabolizing 5-fluorocytosine (5-FC) to the chemotherapeutic agent 5-fluorouracil (5-FU), carboxylesterase (CA), deoxycytidine kinase (dCK), purine nucleoside phosphorylase (PNP), thymidine phosphorylase (TP), thymidine kinase (TK) or xanthine-guanine phosphoribosyl transferase (XGPRT). This type of transgene may also be used to confer a bystander effect.

Any gene or coding sequence of therapeutic relevance can be used in the practice of the invention. For example, genes encoding immunogenic polypeptides, toxins, immunotoxins and cytokines are useful in the practice of the invention. Additional transgenes that may be introduced into a vector of the invention include a factor capable of initiating apoptosis, antisense or ribozymes, which among other capabilities may be directed to mRNAs encoding proteins essential for proliferation, such as structural proteins, transcription factors, polymerases, etc., viral or other pathogenic proteins, where the pathogen proliferates intracellularly, cytotoxic proteins, e.g., the chains of diphtheria, ricin, abrin, etc., genes that encode an engineered cytoplasmic variant of a nuclease (e.g., RNase A) or protease (e.g., trypsin, papain, proteinase K, carboxypeptidase, etc.), chemokines, such as MCP3 alpha or MIP-1, pore-forming proteins derived from viruses, bacteria, or mammalian cells, fusgenic genes, chemotherapy sensitizing genes and radiation sensitizing genes. Other genes of interest include cytokines, antigens, transmembrane proteins, and the like, such as IL-1, IL-2, IL-4, IL-5, IL-6, IL-10, IL-12, IL-18 or flt3, GM-CSF, G-CSF, M-CSF, IFN-α, -β, -γ, TNF-α, -β, TGF-α, -β, NGF, MDA-7 (Melanoma differentiation associated gene-7, mda-7/interleukin-24), and the like. Further examples include, proapoptotic genes such as Fas, Bax, Caspase, TRAIL, Fas ligands, nitric oxide synthase (NOS) and the like; fusion genes which can lead to cell fusion or facilitate cell fusion such as V22, VSV and the like; tumor suppressor gene such as p53, RB, p16, p17, W9 and the like; genes associated with the cell cycle and genes which encode anti-angiogenic proteins such as endostatin, angiostatin and the like.

Other opportunities for specific genetic modification include T cells, such as tumor infiltrating lymphocytes (TILs), where the TILs may be modified to enhance expansion, enhance cytotoxicity, reduce response to proliferation inhibitors, enhance expression of lymphokines, etc. One may also wish to enhance target cell vulnerability by providing for expression of specific surface membrane proteins, e.g., B7, SV40 T antigen mutants, etc.

Additional genes include the following: proteins that stimulate interactions with immune cells such as B7, CD28, MHC class I, MHC class II, TAPs, tumor-associated antigens such as immunogenic sequences from MART-1, gp 100(pmel-17), tyrosinase, tyrosinase-related protein 1, tyrosinase-related protein 2, melanocyte-stimulating hormone receptor, MAGE1, MAGE2, MAGE3, MAGE12, BAGE, GAGE, NY-ESO-1, β-catenin, MUM-1, CDK-4, caspase 8, KIA 0205, HLA-A2R1701, α-fetoprotein, telomerase catalytic protein, G-250, MUC-1, carcinoembryonic protein, p53, Her2/neu, triosephosphate isomerase, CDC-27, LDLR-FUT, telomerase reverse transcriptase, PSMA, cDNAs of antibodies that block inhibitory signals (CTLA4 blockade), chemokines (MIP1α, MIP3α, CCR7 ligand, and calreticulin), anti-angiogenic genes include, but are not limited to, genes that encode METH-1, METH -2, TrpRS fragments, proliferin-related protein, prolactin fragment, PEDF, vasostatin, various fragments of extracellular matrix proteins and growth factor/cytokine inhibitors, various fragments of extracellular matrix proteins which include, but are not limited to, angiostatin, endostatin, kininostatin, fibrinogen-E fragment, thrombospondin, tumstatin, canstatin, restin, growth factor/cytokine inhibitors which include, but are not limited to, VEGF/VEGFR antagonist, sFlt-1, sFlk, sNRP1, angiopoietin/tie antagonist, sTie-2, chemokines (IP-10, PF-4, Gro-beta, IFN-gamma (Mig), IFNα, FGF/FGFR antagonist (sFGFR), Ephrin/Eph antagonist (sEphB4 and sephrinB2), PDGF, TGFβ and IGF-1. Genes suitable for use in the practice of the invention can encode enzymes (such as, for example, urease, renin, thrombin, metalloproteases, nitric oxide synthase, superoxide dismutase, catalase and others known to those of skill in the art), enzyme inhibitors (such as, for example, alpha1-antitrypsin, antithrombin III, cellular or viral protease inhibitors, plasminogen activator inhibitor-1, tissue inhibitor of metalloproteases, etc.), the cystic fibrosis transmembrane conductance regulator (CFTR) protein, insulin, dystrophin, or a Major Histocompatibility Complex (MHC) antigen of class I or II. Also useful are genes encoding polypeptides that can modulate/regulate expression of corresponding genes, polypeptides capable of inhibiting a bacterial, parasitic or viral infection or its development (for example, antigenic polypeptides, antigenic epitopes, and transdominant protein variants inhibiting the action of a native protein by competition), apoptosis inducers or inhibitors (for example, Bax, Bc12, Bc1X and others known to those of skill in the art), cytostatic agents (e.g., p21, p16, Rb, etc.), apolipoproteins (e.g., ApoAI, ApoAIV, ApoE, etc.), oxygen radical scavengers, polypeptides having an anti-tumor effect, antibodies, toxins, immunotoxins, markers (e.g., beta-galactosidase, luciferase, etc.) or any other genes of interest that are recognized in the art as being useful for treatment or prevention of a clinical condition. Further therapeutic genes include a polypeptide which inhibits cellular division or signal transduction, a tumor suppressor gene (such as, for example, p53, Rb, p73), a polypeptide which activates the host immune system, a tumor-associated antigen (e.g., MUC-1, BRCA-1, an HPV early or late antigen such as E6, E7, L1, L2, etc), optionally in combination with a cytokine gene.

The invention further comprises combinations of two or more transgenes with synergistic or complementary activities and nonoverlapping toxicities.

In the vectors of the invention, a transgene/therapeutic gene or coding sequence therefore is under the control of a heterologous or native promoter alone or promoter plus enhancer, i.e. a PIN1 TRE. Exemplary promoters that may be employed include, but are not limited to, adenoviral promoters, such as the adenoviral major late promoter and/or the E3 promoter; promoters such as the cytomegalovirus (CMV) promoter; the Rous Sarcoma Virus (RSV) promoter; inducible promoters, such as the MMT promoter, the metallothionein promoter; heat shock promoters; the albumin promoter; the ApoAI promoter; and tissue-specific TREs or cell status specific TREs such as those described herein or otherwise known to those of skilled in the art.

Therapeutic Methods

An effective amount of a PIN1 TRE-containing vector is administered to an individual as a composition in a pharmaceutically acceptable excipient, examples of which include, but are not limited to, saline solutions, suitable buffers, preservatives, stabilizers, and may be administered in conjunction with suitable agents such as antiemetics. An effective amount is an amount sufficient to effect beneficial or desired results, including clinical results. An effective amount can be administered in one or more administrations. For purposes of this invention, an effective amount of vector is an amount that is sufficient to palliate, ameliorate, stabilize, reverse, slow or delay the progression of the disease state. The amount to be given will be determined by the condition of the individual, the extent of disease, the route of administration, the number of doses administered, and may be adjusted from time to time to achieve maximum efficacy.

Delivery of vectors of the invention is generally accomplished by either site-specific injection or intravenous injection. Site-specific injections of vector may include, for example, injections into tumors, as well as intraperitoneal delivery to the bladder, intrapleural, intrathecal, intra-arterial, subcutaneous or topical application.

Viral vectors may be delivered to the target cell in a variety of ways, including, but not limited to, liposomes, general transfection methods that are well known in the art (such as calcium phosphate precipitation or electroporation), direct injection, and intravenous infusion. The means of delivery will depend in large part on the particular vector (including its form) as well as the type and location of the target cells (i.e., whether the cells are in vitro (i.e. ex vivo) or in vivo).

In a further aspect of the invention, a pharmaceutical composition comprising the recombinant viral vectors and/or viral particles of the invention and a pharmaceutically acceptable carrier is provided. Such compositions, which can comprise an effective amount of a vector of the invention and/or viral particles of the invention in a pharmaceutically acceptable carrier, are suitable for local or systemic administration to individuals in unit dosage forms, sterile parenteral solutions or suspensions, sterile non-parenteral solutions or oral solutions or suspensions, oil in water or water in oil emulsions and the like. Formulations for parenteral and non-parenteral drug delivery are known in the art. Compositions also include lyophilized and/or reconstituted forms of the cancer-specific vector or particles of the invention. Acceptable pharmaceutical carriers are, for example, saline solution, protamine sulfate (Elkins-Sinn, Inc., Cherry Hill, N. J.), water, aqueous buffers, such as phosphate buffers and Tris buffers, or Polybrene (Sigma Chemical, St. Louis Mo.) and phosphate-buffered saline and sucrose. The selection of a suitable pharmaceutical carrier is deemed to be apparent to those skilled in the art from the teachings contained herein. These solutions are sterile and generally free of particulate matter other than the desired cancer-specific vector. The compositions may contain pharmaceutically acceptable auxiliary substances as required to approximate physiological conditions such as pH adjusting and buffering agents, toxicity adjusting agents and the like, for example sodium acetate, sodium chloride, potassium chloride, calcium chloride, sodium lactate, etc. Excipients that enhance uptake of the cancer-specific vector by cells may be included.

If used as a packaged adenovirus, adenovirus vectors may be administered in an appropriate physiologically acceptable carrier at a dose of about 10⁴ to about 10¹⁴ viral particles. If administered as a polynucleotide construct (i.e., not packaged as a virus) about 0.01 ug to about 1000 ug of an adenoviral vector can be administered. The exact dosage to be administered is dependent upon a variety of factors including the age, weight, and sex of the patient, and the size and severity of the tumor being treated. The adenoviral vector(s) may be administered one or more times, depending upon the intended use and the immune response potential of the host, and may also be administered as multiple, simultaneous injections. If an immune response is undesirable, the immune response may be diminished by employing a variety of immunosuppressants, or by employing a technique such as an immunoadsorption procedure (e.g., immunoapheresis) that removes adenovirus antibody from the blood, so as to permit repetitive administration, without a strong immune response. If packaged as another viral form, such as HSV, an amount to be administered is based on standard knowledge about that particular virus (which is readily obtainable from, for example, published literature) and can be determined empirically.

In one embodiment the host organism is a human patient. For human patients, if a therapeutic gene is included in the vector, the therapeutic gene may be of human origin although genes of closely related species that exhibit high homology and are biologically identical or have equivalent function in humans may be used if the gene does not produce an adverse immune reaction in the recipient. A therapeutically active amount of a nucleic acid sequence or a therapeutic gene is an amount effective at dosages and for a period of time necessary to achieve the desired result. This amount may vary according to various factors including but not limited to sex, age, weight of a subject, and the like.

Embodiments of the present invention include methods for the administration of combinations of a cancer-specific vector and a second anti-neoplastic therapy, which may include radiation, administration of an anti-neoplastic agent, etc., to an individual with neoplasia, as detailed in U.S. Patent Application Publication No. 2003-0068307. The cancer-specific vector and anti-neoplastic (chemotherapeutic) agent may be administered simultaneously or sequentially, with various time intervals for sequential administration. In some embodiments, an effective amount of vector and an effective amount of at least one chemotherapeutic agent are combined with a suitable excipient and/or buffer solutions and administered simultaneously from the same solution by any of the methods listed herein or those known in the art. This may be applicable when the chemotherapeutic agent does not compromise the viability and/or activity of the vector itself. Where more than one chemotherapeutic agent is administered, the agents may be administered together in the same composition; sequentially in any order, or alternatively, administered simultaneously in different compositions. If the agents are administered sequentially, administration may further comprise a time delay. Sequential administration may be in any order. The interval between administration of the vector and chemotherapeutic agent may be in terms of at least (or, alternatively, less than) minutes, hours, or days. Sequential administration also encompasses administration of a chosen chemotherapeutic agent followed by the administration of the vector. The interval between administrations may be in terms of minutes, hours, or days.

Administration of the above-described methods may also include repeat doses or courses of a vector of the invention alone or in combination with a chemotherapeutic agent depending, inter alia, upon the individual's response and the characteristics of the individual's disease. Repeat doses may be undertaken immediately following the first course of treatment (i.e., within one day), or after an interval of days, weeks or months to achieve and/or maintain suppression of tumor growth. A particular course of treatment according to the above-described methods, for example, combined cancer-specific vector and chemotherapy, may later be followed by a course of combined radiation and cancer-specific vector therapy, etc.

Anti-neoplastic (chemotherapeutic) agents include those from each of the major classes of chemotherapeutics, including but not limited to: alkylating agents, alkaloids, antimetabolites, anti-tumor antibiotics, nitrosoureas, hormonal agonists/antagonists and analogs, immunomodulators, photosensitizers, enzymes and others. In some embodiments, the antineoplastic is an alkaloid, an antimetabolite, an antibiotic or an alkylating agent. In certain embodiments the antineoplastic agents include, for example, thiotepa, interferon alpha-2a, and the M-VAC combination (methotrexate-vinblastine, doxorubicin, cyclophosphamide). Preferred antineoplastic agents include, for example, 5-fluorouracil, cisplatin, 5-azacytidine, and gemcitabine. Particularly preferred embodiments include, but are not limited to, 5-fluorouracil, gemcitabine, doxorubicin, miroxantrone, mitomycin, dacarbazine, carmustine, vinblastine, lomustine, tamoxifen, docetaxel, paclitaxel or cisplatin. The specific choice of both the chemotherapeutic agent(s) is dependent upon, inter alia, the disease under treatment.

In addition to the use of a single chemotherapeutic (also referred to herein as an “antineoplastic”) agent in combination with a particular vector of the invention, the invention also includes the use of more than one agent in conjunction with the vector of the invention. These combinations of antineoplastics when used to treat neoplasia are often referred to as combination chemotherapy and are often part of a combined modality treatment which may also include surgery and/or radiation, depending on the characteristics of the disease.

There are a variety of delivery methods for the administration of antineoplastic agents, which are well known in the art, including oral and parenteral methods.

Assessment of the efficacy of a particular treatment regimen may be determined by any of the techniques known in the art, including diagnostic methods such as imaging techniques, analysis of serum tumor markers, biopsy, the presence, absence or amelioration of tumor associated symptoms. It will be understood that a given treatment regime may be modified, as appropriate, to maximize efficacy.

Screening Agents and Assays

The invention also provides for screening candidate drugs to identify agents useful for modulating the expression of PIN1 in cancer tissue and useful for treating cancer. Appropriate host cells are those in which the regulatory region of PIN1 is capable of functioning. In one embodiment, a PIN1 TRE is used to make a variety of expression vectors to express a marker that can then be used in screening assays. The expression vectors may be either self-replicating extrachromosomal vectors or vectors that integrate into a host genome. Generally, these expression vectors include a transcriptional and translational regulatory nucleic acid sequence of PIN1 operatively linked to a nucleic acid encoding a marker. The marker may be any protein that can be readily detected. It may be detected on the basis of light emission, such as luciferase and GFP, color, such as β-galactosidase, enzyme activity, such as alkaline phosphatase or antibody reaction, such as a protein for which an antibody exists. In addition, the marker system may be a vector or viral particle of the present invention.

The present invention further provides a method that utilizes host cells transduced with a viral vector comprising a PIN1 TRE of the invention operatively linked to an essential viral gene, e.g. E1a, for screening compounds useful for modulating the expression of PIN1 in cancer tissue. According to this method, a candidate compound is added to the host cells and expression of the essential viral gene or viral replication is detected and compared to a control. Methods for the detection of viral gene expression or viral replication are known in the art.

The various methods of the invention will be described below. Although particular methods of tumor suppression are exemplified in the discussion below, it is understood that any of a number of alternative methods, including those described above are equally applicable and suitable for use in practicing the invention. It will also be understood that an evaluation of the vectors and methods of the invention may be carried out using procedures standard in the art, including the diagnostic and assessment methods described above.

In one embodiment, the viral vector or particle is used to assess the modulation of the PIN1 TRE. According to this embodiment, an effective amount of the viral vectors or viral particles of the invention is contacted with said cell population under conditions where the viral vectors or particles can transduce the neoplastic cells in the cell population, replicate, and kill the neoplastic cells. The candidate agent is either present in the culture medium for the test sample or absent for the control sample. The LD50 of the viral vectors or particles in the presence and absence of the candidate agent is compared to identify the candidate agents that modulate the expression of the PIN1 gene. If the LD50 is different as compared to similar viral vector controls lacking the PIN1 TRE, the candidate agent is capable of modulating the expression of PIN1 and if the LD50 is increased, the agent is a candidate for treating cancers involving this gene and for further development of active agents on the basis of the candidate agent so identified. If the LD50 is decreased, the agent may be a candidate for a treatment combination using the agent and the cancer-specific viral vector.

In a second embodiment, the candidate agent is added to host cells containing the expression vector and the level of expression of a marker is compared with a control. If the level of expression is different, the candidate agent is capable of modulating the expression of PIN1 and if expression is decreased, the agent is a candidate for treating cancers involving this gene and for further development of active agents on the basis of the candidate agent so identified.

Active agents so identified may also be used in combination treatments with a cancer-specific vector of the invention.

Having identified the PIN1 gene as being associated with cancer, a variety of assays may be executed. In an embodiment, assays may be run on an individual gene or protein level. That is, having identified a gene as up-regulated in cancer, candidate bioactive agents may be screened to modulate this gene's response; preferably to down-regulate the gene, although in some circumstances to up regulate the gene. “Modulation” thus includes both an increase and a decrease in gene expression. The preferred amount of modulation will depend on the original change of the gene expression in normal versus tumor tissue, with changes of at least 10%, preferably 50%, more preferably 100-300%, and in some embodiments 300-1000% or greater. Thus, if a gene exhibits a 4 fold increase in tumor compared to normal tissue, a decrease of about four fold is desired; a 10 fold decrease in tumor compared to normal tissue gives a 10 fold increase in expression for a candidate agent is desired.

Candidate agents encompass numerous chemical classes, though typically they are organic molecules, preferably small organic compounds having a molecular weight of more than 100 and less than about 2,500 daltons. Preferred small molecules are less than 2000, or less than 1500 or less than 1000 or less than 500 daltons. Candidate agents comprise functional groups necessary for structural interaction with proteins, particularly hydrogen bonding, and typically include at least an amine, carbonyl, hydroxyl or carboxyl group, preferably at least two of the functional chemical groups. The candidate agents often comprise cyclical carbon or heterocyclic structures and/or aromatic or polyaromatic structures substituted with one or more of the above functional groups. Candidate agents are also found among biomolecules including peptides, saccharides, fatty acids, steroids, purines, pyrimidines, derivatives, structural analogs or combinations thereof. Particularly preferred are peptides.

Candidate agents are obtained from a wide variety of sources including libraries of synthetic or natural compounds. For example, numerous means are available for random and directed synthesis of a wide variety of organic compounds and biomolecules, including expression of randomized oligonucleotides. Alternatively, libraries of natural compounds in the form of bacterial, fungal, plant and animal extracts are available or readily produced. Additionally; natural or synthetically produced libraries and compounds are readily modified through conventional chemical, physical and biochemical means. Known pharmacological agents may be subjected to directed or random chemical modifications, such as acylation, alkylation, esterification, and amidification to produce structural analogs.

All publications and patent applications cited in this specification are herein incorporated by reference as if each individual publication or patent application were specifically and individually indicated to be incorporated by reference.

The present invention has been described in terms of particular embodiments found or proposed by the present inventor to comprise preferred modes for the practice of the invention. It will be appreciated by those of skill in the art that, in light of the present disclosure, numerous modifications and changes can be made in the particular embodiments exemplified without departing from the intended scope of the invention. For example, due to codon redundancy, changes can be made in the underlying DNA sequence without affecting the protein sequence. Moreover, due to biological functional equivalency considerations, changes can be made in protein structure without affecting the biological action in kind or amount. All such modifications are intended to be included within the scope of the appended claims.

The following examples are put forth so as to provide those of ordinary skill in the art with a complete disclosure and description of how to make and use the present invention, and are not intended to limit the scope of what the inventors regard as their invention nor are they intended to represent that the experiments below are all or the only experiments performed. Efforts have been made to ensure accuracy with respect to numbers used (e.g., amounts, temperature, etc.) but some experimental errors and deviations should be accounted for. Unless indicated otherwise, parts are parts by weight, molecular weight is weight average molecular weight, temperature is in degrees Centigrade, and pressure is at or near atmospheric. The following examples are offered by way of illustration and not by way of limitation.

### EXPERIMENTAL

### EXAMPLE 1

Conditionally replicating oncolytic adenoviruses, OV1158 and OV1159, are constructed by replacing the promoter of an essential adenoviral transcription unit, E1A with a human prolyl isomerase 1 (Pin1) promoter. OV1158 and OV1159 are generated by homologous recombination of modified left and right hand sides of the Adenovirus type 5 (Ad5) genome in eukaryotic cells.

Modifications to the left hand side of the Ad5 genome are made in plasmid pXC-1 (Microbix). pXC-1 contains nucleotides 22 to 5790 of the Ad5 genome in a pBR322 backbone. Modifications to pXC-1 to generate platform plasmids have been described, previously (Yu et al. Cancer Res., 59: 1498-1504, 1999). Briefly, unique Age I and Eag I sites are introduced in pXC-1 to facilitate insertion of heterologous elements. An Age I site is introduced in pXC-1 (between the E1A mRNA cap site and the E1A translation initiation site), by insertion of a thymidine at position 552, to generate plasmid CN95. The Eag I site present in the pBR322 backbone of pXC-1 is separately removed by Eag I digestion, mung bean nuclease treatment and religation of the treated vector to generate plasmid CN114. Overlapping PCR products are then produced using the primer sets 15.133A/9.4 and 9.3/24.020 (see Table 2) and the template CN95. Amplification of the overlapping PCR products with the flanking primers 15.133A and 24.020 produces a final PCR product which is digested with EcoR I and Kpn I and ligated into the similarly cut CN114. The resulting plasmid, CN124, has a unique Eag I site between the E1B promoter and the E1B mRNA cap site as well as the Age I site described in CN95. To remove the E1A promoter from CN124, a PCR fragment is amplified from CN124 with the primer set CN124U/CN124L. The amplified fragment is digested with EcoR I and Age I, then ligated into the similarly cut CN124 to generate CN306. In CN306, the E1A promoter is deleted via a 68-nucleotide deletion upstream of the E1A cap site and the Hind III site of the pBR322 backbone is replaced with an Xho I site.

As a platform plasmid CN306 serves as the basis for several downstream modifications to the original pXC-1 plasmid. In the case of CP1486, a heterologous self-processing cleavage site, 2A, is adapted from the Foot and Mouth Disease Virus (FMDV 2A) linking the E1A transcription unit and the E1B 55k coding region in a single open reading frame transcriptionally driven by a previously introduced human E2f-1 promoter. See, e.g., U.S. patent application Ser. No. 10/857,498. Briefly, the human E2f-1 promoter is amplified from human genomic DNA and flanked with Age I sites using the primer set 1405.77.1/1405.77.2. The resulting PCR product is digested with Age I and introduced into the unique Age I site attributed to CN306. Truncated sections of the E1A transcription unit, the FMDV 2A oligopeptide and the E1B 55k coding region are amplified with the primer sets 1460.138.3/1460.138.4, 1460.138.1/1460.138.2, and 1460.138.5/1460.138.6, respectively. Each primer set introduces flanking restriction sites such that the E1A fragment has Xba I and Sal I ends, the FMDV 2A fragment has Sal I ends, and the E1B 55k fragment has Sal I and Hind III ends. The fragments are then subcloned into a single shuttle vector with the complementary sites such that the E1A transcription unit (stop codon removed) is immediately adjacent to the FMDV 2A oligopeptide which precedes the initiation codon of E1B 55k with all three being in frame. The final subcloned fragment is then released from the shuttle vector by Xba I and Hind III restriction digestion and introduced into the aforementioned CN306-derived vector containing the E2f-1 promoter inserted at the Age I site.

The resulting plasmid, CP1486, serves as one of the parent plasmids for the introduction of the human prolyl isomerase 1 (PIN1) promoter variants into the left-hand side of the Ad5 genome.

Another variant of pXC-1, CP1369, also serves as a parent plasmid for the introduction of the PIN1 promoter into the left-hand side of the Ad5 genome. In CP1369, a human cytomegalovirus (hCMV) promoter, human granulocyte macrophage colony stimulating factor (hGM-CSF) cDNA, and bovine growth hormone (BGH) poly A tail is introduced as a cassette downstream of the E1B transcription unit. The human GM-CSF cDNA is initially released from plasmid pGT60-hGM-CSF (Invivogen, San Diego, Calif.) by BamH I and EcoR I digestion and introduced into the similarly cut pcDNA 3 (Invitrogen, Carlsbad, Calif.) to form a hCMV-hGM-CSF-BGH poly A cassette in plasmid CP1367. A pXC-1 derivative, plasmid CP1366, was generated in parallel in which Pac I and Xho I restriction sites were introduced downstream of the E1B 55k stop codon. To introduce the Pac I and Xho I sites, primer sets YC1/YC3 and YC2/YC4 are used to amplify overlapping segments of a pXC-1 derived backbone. Mixing of the amplified fragments followed by a second round of PCR using the flanking primer set YC3/YC4 produces a fragment containing the Pac I and Xho I restriction sites. This fragment is cleaved with Hpa I and Afl II, then ligated into the similarly cut pXC-1 to yield CP1366. The hCMV-hGM-CSF-BGH poly A cassette from CP1367 is then introduced into plasmid CP1366 via the Pac I site to generate plasmid CP1369.

Plasmids CP1486 and CP1369 serve as parent plasmids for the introduction of the human PIN1 promoter into the left-hand side of the Ad5 genome. To amplify variants of the PIN1 promoter, A549 genomic DNA is isolated using a DNeasy Tissue Kit (Qiagen, Valencia, Calif.). Variants of the PIN1 promoter are amplified from the isolated human genomic DNA of the PIN1 by PCR with the primer sets 1618.83.1/1618.83.3 and 1618.83.2. PCR amplification with the primer set 1618.83.1/1618.83.3 yields an ˜400 nucleotide fragment with Age I flanking ends. Likewise, PCR amplification with the primer set 1618.83.2/1618.83.3 yields an ˜300 nucleotide fragment with Age I flanking ends.

For initial characterization of the PIN1 promoter and its variants, the desired platform is the PIN1 promoter (or variants) introduced at the Age I site described for CN306 and the wild-type E1B promoter and transcription unit. To generate such a vector, it is necessary to reconstitute portions of the original pXC-1 plasmid downstream of the Xba I site in CP1486. Therefore, CP1486 is digested with Age I, Xba I and Hind III. From that cut, an ˜7 kb vector is isolated as well as the fragment from the downstream Age I site to the Xba I site. CP1369 is then digested with Xba I and Hind III and the ˜1.5 kb insert fragment isolated. Each of the amplified PIN1 promoter variants are then digested with Age I. The fragments are then assembled via a 4-way ligation to generate the constructs CP1521 and CP1522. CP1521 has an ˜300 nucleotide variant of the PIN1 promoter driving E1A transcription and a reconstituted wild-type pXC-1 sequence downstream of the Xba I site. Likewise, CP1522 has an ˜400 nucleotide variant of the PIN1 promoter driving E1A transcription and a reconstituted wild-type pXC-1 sequence downstream of the Xba I site.

To generate recombinant adenoviruses with E1A under the transcriptional control of the human PIN1 promoter, CP1521 (SEQ ID NO:68) and CP1522 (SEQ ID NO:69) are separately co-transfected with plasmid pBHGE3 (Microbix, Toronto, Ontario, Canada) into 293 (Microbix) or A549 clone 51 cells (Farson et al. Molecular Therapy, Vol 9 Supplement 1: p. S294, Abstract No. 775, 2004 and U.S. patent application Ser. No. 10/613,106) as described, previously (Rodriguez et al. Cancer Res., 57: 2559-2563, 1997). Briefly, pBHGE3 contains the Ad5 genome with the exception of the nucleotides between 188 to 1339, in a pBR322 backbone. Linearization of pBGHE3 and pXC-1 derivatives followed by co-transfection into cells permissive to and in some cases trans-complementary to adenovirus replication results in homologous recombination yielding replication competent adenoviruses. In this case, CP1521 and pBHGE3 are linearized and co-transfected into 293 cells. Cells are scraped into the supernatant and collected 13 days post-transfection, subjected to freeze-thaw lysis and plated onto A549 cells at several dilutions under a solid media overlay. 6 days after plating out the lysates, plaques are picked, diluted, and replated onto A549 cells under a solid media overlay. 7 days after this second plating, plaques are isolated and used to infect A549 cells to generate small viral stocks for characterization and further amplification. The resulting virus has been designated OV1158.

CP1522 is similarly co-transfected with pBHGE3. However, A549 clone 51 cells are transfected rather than 293 cells. All steps subsequent to transfection are otherwise identical. The resulting virus has been designated OV1159.

TABLE 2
Primer sequences (5′-3′)

| | | |
|---|---|---|
| 15.133A | TCGTCTTCAAGAATTCTCA | (SEQ ID NO:47) |
| 9.4 | GTATATAATGCGGCCGTGGGC | (SEQ ID NO:48) |
| 9.3 | GCCCACGGCCGCATTATATAC | (SEQ ID NO:49) |
| 24.020 | CCAGAAAATCCAGCAGGTACC | (SEQ ID NO:50) |
| CN124U | AGCTGAATTCTCGAGTTGGAGCCA | (SEQ ID NO:51) |
| | CTATCGACTACG | |
| CN124L | AGCTACCGGTCACGTAAACGGTCA | (SEQ lID NO:52) |
| | AAGTCC | |
| 1405.77.1 | ATACCGGTGGTACCATCCGGACAA | (SEQ ID NO:53) |
| | AGCCTGCGCG | |
| 1405.77.2 | AGACCGGTCGAGGGCTCGATCCCG | (SEQ ID NO:54) |
| | CTCCG | |
| 1460.138.1 | ATGCAGCGTCGACGCTCCAGTAAA | (SEQ ID NO:55) |
| | GCAGACTCTA | |
| 1460.138.2 | CATGATCGTCGACTGGACCTGGGT | (SEQ ID NO:56) |
| | TGCTCTCAAC | |
| 1460.138.3 | TGTGTCTAGAGAATGCAATAG | (SEQ ID NO:57) |
| 1460.138.4 | GATATATGTCGACTGGCCTGGGGC | (SEQ ID NO:58) |
| | GTTTACAGC | |
| 1460.138.5 | GACATGCGTCGACATGGAGCGAAG | (SEQ ID NO:59) |
| | AAACCCATCTG | |
| 1460.138.6 | CCATAGAAGCTTACACCGTGTAG | (SEQ ID NO:60) |
| YC1 | CCGCTCGAGCGGTTAATTAACCAC | (SEQ ID NO:61) |
| | CTCAATCTGTATCTTCAT | |
| YC2 | GGTTAATTAACCGCTCGAGCGGAC | (SEQ ID NO:62) |
| | TGAAATGTGTGGGCGTGG | |
| YC3 | TGAGACGCCCGACATCACCT | (SEQ ID NO:63) |
| YC4 | TGGCTGCAGCGGCTGAAGC | (SEQ ID NO:64) |
| 1618.83.1 | ATGCGACCGGTCGGCATTAGCCAA | (SEQ ID NO:65) |
| | TCCATAAG | |
| 1618.83.2 | ATGCGACCGGTAAGGGGTCGGGAG | (SEQ ID NO:66) |
| | TTTTTTGG | |
| 1618.83.3 | ATGCGACCGGTCTCAGCTGCGCCG | (SEQ ID NO:67) |
| | CCTGTCGC | |

It will be appreciated that the methods and compositions of the instant invention can be incorporated in the form of a variety of embodiments, only a few of which are disclosed herein. It will be apparent to the artisan that other embodiments exist and do not depart from the spirit of the invention. Thus, the described embodiments are illustrative and should not be construed as restrictive.

What is claimed is:

1. An isolated prolyl isomerase (PIN1) polynucleotide selected from the group consisting of sequences comprising SEQ ID NO:1, nucleotides 1818 to 2221 of SEQ ID NO:1, nucleotides 1924 to 2221 of SEQ ID NO:1, nucleotides 1854 to 2221 of SEQ ID NO:1, nucleotides 5 to 297 of SEQ ID NO:45 and nucleotides 7 to 374 of SEQ ID NO:46, wherein said polynucleotide preferentially directs gene expression in cancer cells.

2. An isolated PIN1 polynucleotide according to claim 1, wherein said polynucleotide comprises nucleotides 1818 to 2221 of SEQ ID NO:1.

3. An isolated PIN1 polynucleotide according to claim 1, wherein said polynucleotide comprises nucleotides 1924 to 2221 of SEQ ID NO:1.

4. An isolated PIN1 polynucleotide according to claim 1, wherein said polynucleotide comprises nucleotides 5 to 297 of SEQ ID NO:45.

5. An isolated PIN1 polynucleotide according to claim 1, wherein said polynucleotide comprises said polynucleotide comprises nucleotides 7 to 374 of SEQ ID NO:46.

6. A replication competent adenovirus vector comprising a cancer specific PIN1 transcriptional regulatory element (TRE) derived from the sequence upstream of the translational start codon for a PIN1 gene, presented herein as SEQ ID NO:1, wherein said adenovirus vector selectively replicates in cancer cells.

7. A replication competent adenovirus vector according to claim 6, wherein said PIN1 TRE consists essentially of SEQ ID NO:1, nucleotides 5 to 297 of SEQ ID NO:45 or nucleotides 7 to 374 of SEQ ID NO:46.

8. A replication competent adenovirus vector according to claim 6, wherein said PIN1 TRE is a fragment of SEQ ID NO:1 and said fragment has tumor selective transcriptional regulatory activity.

9. A replication competent adenovirus vector according to claim 8, wherein said PIN1 TRE is selected from the group consisting of nucleotides, from about 1 to 2221 of SEQ ID NO:1, from about 1818 to 2221 of SEQ ID NO:1, from about 1924 to 2221 of SEQ ID NO:1, from about 1931 to 2221 of SEQ ID NO:1 and from about 1854 to 2221 of SEQ ID NO:1.

10. The adenovirus vector according to claim 6, wherein said adenovirus vector has a first adenovirus gene essential for replication under transcriptional control of said PIN1 TRE.

11. The adenovirus vector according to claim 10, wherein said first adenovirus gene essential for replication is an early gene selected from the group consisting of E1A, E1B, E2A, E2B and E4.

12. The adenovirus vector according to claim 6, wherein the adenoviral vector comprises first and second adenoviral genes co-transcribed under transcriptional control of said PIN1 TRE.

13. The adenovirus vector according to claim 12, further comprising an internal ribosome entry site (IRES).

14. The adenovirus vector according to claim 12, further comprising a self-processing cleavage sequence.

15. The adenovirus vector according to claim 14, wherein said self-processing cleavage sequence is selected from the group consisting of SEQ ID NO: 6 through 37.

16. The adenovirus vector according to claim 10, further comprising a second adenovirus gene essential for replication under transcriptional control of a cell type specific TRE.

17. The adenovirus vector according to claim 16, wherein said cell type specific TRE is selected from the group consisting of a TERT TRE, an uPA TRE, a uPAR TRE, a PRL-3 TRE, an E2F TRE, an EBV-specific TRE, a HRE, an urothelial cell-specific TRE, an uroplakin TRE, a melanocyte cell specific TRE, a MART-1 TRE, TRP-1 TRE, a TRP-2 TRE and a CRG-L2 TRE.

18. The adenovirus vector according to claim 16, wherein said second adenovirus gene essential for replication is an early gene selected from the group consisting of E1A, E1B, E2A, E2B and E4.

19. The adenovirus vector according to claim 1, further comprising a transgene.

20. The adenovirus vector of claim 19, wherein said transgene is operatively linked to PIN1 TRE.

21. The adenovirus vector of claim 19, wherein said transgene is operatively linked to TRE selected from the group consisting of a TERT TRE, an uPA TRE, a uPAR TRE, a PRL-3 TRE, an E2F TRE, an EBV-specific TRE, a HRE, an urothelial cell-specific TRE, an uroplakin TRE, a melanocyte cell specific TRE, a MART-1 TRE, TRP-1 TRE, a TRP-2 TRE and a CRG-L2 TRE.

22. The adenovirus vector according to claim 19, wherein the transgene is cytotoxic.

23. The adenovirus vector according to claim 19, wherein the transgene is a cytokine.

24. The adenovirus vector of claim 23, wherein said cytokine is GM-CSF gene.

25. The adenovirus vector according to claim 22, further comprising a polynucleotide encoding adenoviral death protein (ADP).

26. An isolated host cell comprising the adenovirus vector of claim 1.

27. A pharmaceutical composition comprising the adenovirus vector of claim 1.
